(19) United States
(12) Patent Application Publication
(10) Pub. No.: US 2005/0159364 A1
(43) Pub. Date: Jul. 21, 2005

(54) Copper antagonist compounds

(76) Inventor: Garth J.S. Cooper, Auckland (NZ)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 11/018,720

(22) Filed: Dec. 20, 2004

Related U.S. Application Data
(60) Provisional application No. 60/531,204, filed on Dec. 19, 2003.

(51) Int. Cl. A61K 38/04; A61K 31/66; A61K 31/47; A61K 31/195

(52) U.S. Cl. 514/19; 514/114; 514/566

(57) ABSTRACT

Copper antagonist compounds and the use of such compounds in methods for the treatment, prevention, or amelioration of various disorders that would be benefited by reduction in copper, for example copper (II), including neurodegenerative and other disorders.

### CROSS REFERENCE TO RELATED APPLICATIONS

This application claims the benefit of priority of U.S. Provisional Application No. 60/531,204 filed on Dec. 19, 2003, which is incorporated herein by reference in its entirety.

### FIELD

The invention provides a compound of Formula I or II, and stereoisomers, pharmaceutically acceptable salts and prodrugs thereof, and pharmaceutically acceptable salts of the prodrugs. These compounds bind or chelate copper and are copper antagonists. Notably, the invention includes compounds that are potent and selective antagonists of Cu⁺² and have utility in a variety of therapeutic areas. In particular, the present compounds are of value for the curative or prophylactic treatment of neurodegenerative diseases, disorders, and conditions. The invention also provides pharmaceutical compositions comprising a compound of Formula I and/or II, and to methods of treatment of neurodegenerative disorders, as well as diabetes, insulin resistance, Syndrome X, obesity, diabetic cardiomyopathy, diabetic neuropathy, diabetic nephropathy, diabetic retinopathy, cataracts, hyperglycemia, hypercholesterolemia, hypertension, hyperinsulinemia, hyperlipidemia, atherosclerosis, tissue ischemia, and diseases, disorders or conditions characterized in whole or in part by copper-related tissue damage.

### BACKGROUND

The following description includes information that may be useful in understanding the present invention. It is not an admission that any of the information provided herein is prior art, or relevant, to the presently described or claimed inventions, or that any publication or document that is specifically or implicitly identified is prior art or a reference that may be used in evaluating patentability of the described or claimed inventions.

Neurodegenerative diseases, including Parkinson's Disease and Alzheimer's Disease, are a significant issue in many modern countries with aging populations. For example, Alzheimer's disease (AD) is one of the most common age-related neurodegenerative and complex dementing illness. It affects nearly half of individuals over the age of 85. With the aging of the population it has become a major public health problem due to the increasing prevalence of AD, the long duration of the disease, the high cost of care, and the lack of disease-modifying therapy. AD has been reported to afflict 15 million people worldwide, including 4 million in the United States alone, and has been predicted that this incidence will more than triple in the United States by 2050. See Geriatrics 58 supp:3-14 (2003).

It has also been reported that AD ties with stroke as the third most common cause of death in the United States (Ewbank D. C., Am J Public Health 89:90-92 (1999)) and is a frequently articulated fear of the elderly. Both incidence and prevalence increase sharply with age. See Kawas C., et al., Neurology 54:2072-2077 (2000); Jorm A. F. & Jolley D., Neurology 51:728-733 (1998). When mild cases are included, AD prevalence may be as high as 10.3% in noninstitutionalized white persons older than 65 years of age (Evans D. A., et al., JAMA 262:2551-2556 (1989)), and this figure is potentially even higher for black and Hispanic persons. See Gurland B. J., et al., Int J Geriatr Psychiatry 14:481-493 (1999). With a reported average yearly cost of care of $35,287 per patient (Ernst R. L., et al., Arch Neurol 54:687-693 (1997)), this illness is said to generate an annual cost to the U.S. economy of more than $141 billion (1997 dollars). The Alzheimer's Association reports the average lifetime cost per patient is $174,000. It has been reported that there are currently 4.9 million persons in the United States 85 years of age or older and that of these, 40% (1.8 million) may meet clinical criteria for dementia. It has been suggested that the steady increase in the number of persons living into the ninth and tenth decades of life multiplies the financial implications of this public health problem. See Clark C. M., et al., Ann Int Med 138:400-411 (2003). The emotional and psychological toll on caregivers is also said to be significant. See Geriatrics 58 supp:3-14 (2003).

The onset of AD is gradual and marked by a progressive decline in cognition advancing to the loss of motor function in the later stages of the disease. Early warning symptoms in an AD patient are said to include cognitive and functional decline, particularly loss of the ability to perform activities of daily living, eventually leading to the patient requiring care or a nursing home placement. Behavioral symptoms such as apathy, disturbed mood, agitation, aggression, anxiety, and circadian rhythm reversal, are distressing to both the patient and the caregiver.

The etiology of AD is not completely known, but several characteristic pathological changes have been identified and form the basis for hypotheses relating to the mechanism of onset and progression of AD. According to the neuronal cytoskeletal degeneration hypothesis, cytoskeletal changes are the main events that lead to neurodegeneration in AD, and the hyperphosphorylation and aggregation of tau polypeptide are related to the activation of cell death processes. See De Ferrari G. V. & Inestrosa N. C., Brain Res Brain Res Rev 33:1-12 (2000). Neurofibrillary tangles in themselves are reportedly not sufficient to cause AD, although it may be that cognitive deficits may not occur until neurofibrillary tangles have been formed. See Schonberger S. J., et al., Proteomics 1:1519-1528 (2001). According to the amyloid cascade hypothesis, neurodegeneration in AD begins with the abnormal processing of the amyloid precursor protein (APP) and results in the production, aggregation, and deposition of amyloid β (Aβ). See De Ferrari G. V. & Inestrosa N. C., Brain Res Brain Res Rev 33:1-12 (2000). Amyloid deposits in themselves are said not to be sufficient to cause AD; however Aβ toxicity may occur before plaques are formed when it is in a nonfibrillar form. See Schonberger S. J., et al., Proteomics 1:1519-1528 (2001). The amyloid cascade is hypothesized to facilitate neurofibrillary tangle formulation and cell death. Id.

Senile (beta-amyloid) plaques are the most widely studied neuropathologic changes in AD. Amyloid-containing plaques do not affect the entire nervous system, but rather form primarily in certain vulnerable cortical and subcortical brain regions; the sensory and motor areas tend to remain unaffected. A currently widely held hypothesis of amyloid plaque development proposes that soluble amyloid begins to deposit in a vulnerable area of the cortex, sometimes due to a faulty gene (familial AD) and sometimes for other, as yet undetermined reasons (sporadic AD). The amyloid deposit is thought to trigger a reaction in nearby healthy neurons that leads to the degeneration and death of the healthy neurons. It is thought that vulnerable regions induce the nuclei of various transmitter systems, leading to their degeneration, whereby a healthy neuron originating, for example, in the brain stem may encounter and be adversely affected by the damaged area, leading to degeneration and cell death.

It has been reported that some brain regions show greater degenerative changes in specific neurotransmitters than do other regions. Changes are said to occur in the function of the monoaminergic neural systems that release glutamate, norepinephrine, and serotonin as well as in a few neuropeptide-containing systems. These systems reportedly do not degenerate in all patients simultaneously or to the same degree. However, the pathology is said to be fairly constant. Changes in glucose utilization are said to occur early in the clinical evolution of AD and may reflect subclinical neuropathologic changes. See Geriatrics 58 supp:3-14 (2003). It has also been reported that amyloid accumulation in the cerebral cortex and subsequent inflammatory changes invariably occur in patients who eventually develop AD, sometimes years or decades before clinical symptoms. It has been proposed that this indicates that amyloid deposits precede AD pathology rather than result from it. Id.

It has been proposed that chronic neuroinflammation may be responsible for the degeneration of the basal forebrain cholinergic system in AD via a chain of inflammatory processes, initiated by the accumulation of Aβ deposits, which is said to activate local microglia and astrocytes leading to a release of cytokines and acute-phase proteins. Id. Local neurons and their processes may be injured by these inflammatory changes and by the neurotoxicity of amyloid β (Selkoe D. J., Scient 275:630-631 (1997)) leading to the selective death of cholinergic neurons. See Geriatrics 58 supp:3-14 (2003). It has been asserted that this process in the basal forebrain is marked by the loss of cholinergic neurons, a decline in cholinesterase activity, and the depletion of acetylcholine. Id.

The reported identification of disease-causing autosomal dominant mutations as well as gene polymorphisms that alter the risk for pathology has been suggested to indicate that AD is a genetically complex disorder. The genes that allegedly contribute to AD pathology appear in all cells, but their expression reportedly varies in different areas of the brain and in different individuals. Also, these genes reportedly account for a very small percentage of the total prevalence of AD. Indeed, it is said to be possible for individuals who carry (apolipoprotein) apoE4 alleles to show diffuse amyloid deposits without developing the lesions or symptoms of AD. See Id.

Therapy of AD encompasses attempts at prevention, risk reduction, symptom management, and delay in progression of the disease. Pharmacologic treatment targets include treatment of cognitive symptoms, for which the cholinesterase inhibitors have been proposed; treatment for behavioral disturbances such as delusions, agitation and aggression, which have been treated with antipsychotic agents and anticonvulsants, reportedly with moderate success; and treatment for depression, for which selective serotonin reuptake inhibitors (SSRIs) and other antidepressant agents have been said to be somewhat successful. See Id.

Other pharmacologic treatments include anticonvulsant drugs, particularly carbamazepine and valproic acid which have reportedly met with some success, but may be limited by adverse side effects. Beta-blockers, antidepressants, lithium, benzodiazepines, and other drugs have reportedly produced inconsistent results, and it is thought many of these drugs may produce sedation, worsen cognitive function, and increase the risk for falls. See Mayeux R. & Sano M., “Treatment of alzheimer's disease.” N Engl J Med 341:1670-1679 (1999). It has been reported that tricyclic antidepressant drugs have anticholinergic activity and can cause confusion or orthostatic hypotension. See Geriatrics 58 supp:3-14 (2003).

Cholinesterase inhibitors (ChE-I), often in conjunction with high-dose vitamin E, are said to represent current approved options for treating mild-to-moderate AD. See Doody R S, et al., Neurology 56:1154-1166 (2001). The three agents in common use (donepezil, rivastigmine, and galantamine) reportedly help cognition, function, and behavior in short-term placebo-controlled studies as well as in longer placebo-controlled studies up to 1 year in duration and in open-label extensions for up to 3 years. See, for example, Rogers S. L., et al., Neurology 50:136-145 (1998); Doody R. S., et al., Arch Neurol 58:427-433 (2001); Farlow M., et al., Eur Neurol 44:236-241 (2000); Corey-Bloom J., et al., Psychopharmacol 1:55-65 (1998); Raskind M. A., et al., Neurology 54:2261-2268 (2000); Winblad B., et al., Neurology 57:489-495 (2001); Doody R. S. & Kershaw P., Neurology 56 (suppl 3):A456 (2001); Mohs R. C., et al., Neurology 57:481-488 (2001); Corey-Bloom J., et al., Psychopharmacol 1:55-65 (1998); Feldman H., et al., Neurology 57:613-620 (2001); Tariot P. N, et al., Neurology 54:2269-2276 (2000); Farlow M., et al., Eur Neurol 44:236-241 (2000).

While ChE-I have been said to have positive effects on cognitive, functional, and behavioral outcomes in mild-to-moderate and possibly severe stages of AD during short- and long-term treatment, and reportedly are generally well tolerated, reported limitations include that these most widely used current treatments for AD target only one aspect of this complex disorder, the degeneration of cholinergic neurons and that improvements from baseline are at best moderate and may not be sustained for the full duration of the disease. Adverse events are said to be significant for some patients and include gastrointestinal disturbances, asthenia, dizziness, and headache. There is a need for medications with alternative mechanisms of action, greater efficacy, and improved tolerability. See Geriatrics 58 supp:3-14 (2003).

Others have proposed treatments for AD that target other, noncholinergic pathways: oxidative damage (Ginkgo biloba); inflammation (Ginkgo biloba, nonsteroidal anti-inflammatory drugs (NSAIDs)); glutamatergic neurotransmission and cell death (NMDA-receptor antagonists, e.g., memantine); and serotonergic and dopaminergic disruptions that give rise to disturbing AD behaviors (atypical antipsychotics and SSRIs). See, for example, Le Bars P. L., et al., JAMA 278:1327-1332 (1997); Wettstein A., Phytomedicine 6:393-401 (2000); van Dongen M. C. J. M., et al.,; van Dongen M. C., et al., J Am Geriatr Soc. 48:1183-1194 (2000); Doraiswamy P. M., et al., Neurology 48:1511-1517 (1997); Scharf S., et al., Neurology 53:197-201 (1999); Eighth International Conference on Alzheimer's Disease and Related Disorders. Stockholm, Sweden; July 20-25 (2002); Parsons C. G., et al.; Parsons C. G., et al., Neuropharmacology 38:735-767 (1999); Reisberg B., Ferris S., Neurobiol Aging 23(Suppl 1):S555 (2002) (International Conference on Alzheimer's Disease; July 2002); Ruther E., et al., Pharmacopsychiatry 33:103-108 (2000).

The prevalence of psychosis, depression and agitation is said to be very high among AD patients, and drugs that target the dopaminergic and seratomergic systems have been proposed for the treatment of such patients. See De Deyn P. P., et al., Neurology 53:946-955 (1999); Street J. S., et al., Arch Gen Psychiatry 57:968-976 (2000); Geriatrics 58 supp:3-14 (2003). For agitation in AD, a number of compounds, for example, carbamazepine and divalproex, have reportedly shown some benefit based on the Brief Psychiatric Rating Scale (BPRS) and Clinical Global Impression of Change in cognitive functioning (CGIC) scales. See Tariot P. N., et al., Am J Psychiatry 155:54-61 (1998); Porsteinsson A. P., et al., Am J Geriatric Psychiatry 9:58-66 (2001); Tariot P. N., et al., Curr Ther Res Clin Exp 62:51-67 (2001). See also Pollock B. G., et al., Am J Psychiatry 159:460-465 (2002); Veld B. A., et al., N Engl J Med 345:1515-1521 (2001); Zandi P. P., et al., Neurology 59:880-886 (2002); Lindsay J., et al., Am J Epidemiol 156:445-4530 (2002); Breitner J. C. & Zandi P. P., N Engl J Med 345:1567-1568 (2001).

A role for antioxidants in the treatment and/or prevention of AD has also been assessed. See Sano M., et al., N Engl J Med 336:1216-1222 (1997); Heart Protection Study Collaborative Group, “MRC/BHF Heart Protection Study of antioxidant vitamin supplementation in 20,536 high-risk individuals:a randomized placebo-controlled trial.” Lancet 360:23-33 (2002).

Others have proposed that lipids may play a role in amyloid accumulation and AD. See Jick H., et al. Lancet 356:1627-1631 (2000). Blood levels of homocysteine are reportedly elevated in AD, and hyperhomocysteinemia has also been hypothesized to contribute to AD pathophysiology. See Aisen P. S., et al., Am J Geriatr Psychiatry 11:246-9 (2003). Other proposed therapies for AD include the surgical implanatation of a shunt to drain cerebrospinal fluid from the skull and allow replenishment of normal cerebrospinal fluid; the use of insulin-sensitising compounds as proposed therapeutic agents for cognitive impairment in AD; high intensity light therapy; and human nerve growth factor gene transfer therapy.

It has been reported that amyloid precursor protein (APP) can bind Zn and Cu, and Aβ precipitation and toxicity in AD and abnormal interactions with neocortical metal ions such as Zn, Cu and Fe have also been discussed. See Bush A. I., Trends Neurosci 26:207-214 (2003); White A. R., et al., Brain Res 842:439-444 (1999). Cu binding to APP has been reported to greatly reduce Aβ production in vitro. See Barnham K. J., et al., JBC 278:17401-17407 (2003). Regarding discussion of the ability of Aβ to trap and prevent Cu from participating in radical-generating activity, see Kontush A., et al., Free Radic Biol Med 30:119-128 (2001); Kontush A., et al., Free Radic Res 35:507-517 (2001); Zou K., et al., J Neurosci 22:4833-4841 (2002). Data relating to elevation of Cu in the serum of individuals with AD has been said to provide support for a hypothesis that Aβ directs Cu into the circulation. See Squitti R., et al., Neurology 59:1153-1161 (2002). Others have indicated that the biochemical behaviour of Aβ appears to be pleiotropic:at a high peptide to metal-ion stoichiometry, Aβ can remove metal ion and is protective, while at high metal-ion-to-peptide stoichiometry Aβ becomes aggregated and catalytically pro-oxidant. See Bush A. I., Trends Neurosci 26:207-214 (2003).

Oral treatment with clioquinol (CQ), a retired United States Pharmacopeia antibiotic and orally bioavailable Cu—Zn chelator, was reported to induce a decrease in brain Aβ deposition in a blind study of Tg2576 transgenic mice treated orally for nine weeks. In contrast, treatment of Tg2576 mice with the hydrophilic Cu chelator, triethylenetetramine, reportedly did not inhibit amyloid deposition. See Cherny R. A., et al., Neuron 30:665-676 (2001). It has been contended that, unlike common chelators such as penicillamine, CQ is hydrophobic and crosses the blood brain barrier. The results of the Tg2576 transgenic mouse study above were said to indicate that systemic metal-ion depletion is not likely to be a useful therapeutic strategy for AD. See Bush A. I., Trends Neurosci 26:207-214 (2003).

The complexity of the etiology of AD has presented a number of potential targets for therapeutic and preventative intervention. However, despite intensive research, current AD therapies predominantly target the management and treatment of the symptoms of AD rather than the underlying cause or mechanism, and in any event, reportedly have limited efficacy. There remains a significant need for effective therapeutic and preventative methods for the treatment of AD and other neurological discorders.

### BRIEF SUMMARY

The inventions described and claimed herein have many attributes and embodiments including, but not limited to, those set forth or described or referenced in this Summary. The inventions described and claimed herein are not limited to or by the features or embodiments identified in this Summary, which is included for purposes of illustration only and not restriction.

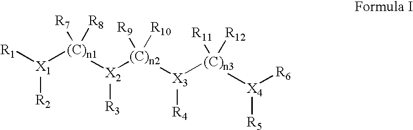

The invention includes acyclic compounds of Formula I for tetra-heteroatom acyclic analogues, where X1, X2, X3, and X4 are independently chosen from the atoms N, S or O such that,

(a) for a four-nitrogen series, i.e., when X1, X2, X3, and X4 are N then:R1, R2, R3, R4, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R3, R4, R5, or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(b) for a first three-nitrogen series, i.e., when X1, X2, X3, are N and X4 is S or O then:R6 does not exist; R1, R2, R3, R4 and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R3, R4, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a second three-nitrogen series, i.e., when X1, X2, and X4 are N and X3 is O or S then:R4 does not exist and R1, R2, R3, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R3, R5, or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(d) for a first two-nitrogen series, i.e., when X2 and X3 are N and X1 and X4 are O or S then:R1 and R6 do not exist; R2, R3, R4, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R2, R3, R4, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(e) for a second two-nitrogen series, i.e., when X1 and X3 are N and X2 and X4 are O or S then:R3 and R6 do not exist; R1, R2, R4, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R4, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(f) for a third two-nitrogen series, i.e., when X1, and X2 are N and X3 and X4 are O or S then:R4 and R6 do not exist; R1, R2, R3, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R3, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(g) for a fourth two-nitrogen series, i.e., when X1 and X4 are N and X2 and X3 are O or S then:R3 and R4 do not exist; R1, R2, R5 and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R5, or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Second, for a tetra-heteroatom series of cyclic analogues, R1 and R6 are joined together to form the bridging group (CR13R14)n4, and X1, X2, X3, and X4 are independently chosen from the atoms N, S or O such that,

(a) for a four-nitrogen series, i.e., when X1, X2, X3, and X4 are N then:R2, R3, R4, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R2, R3, R4, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R7, R8, R9, R10, R11, R12, R13 or R14 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(b) for a three-nitrogen series, i.e., when X1, X2, X3, are N and X4 is S or O then:R5 does nor exist; R2, R3, and R4 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R2, R3 or R4 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, R12, R13 or R14 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a first two-nitrogen series, i.e., when X2 and X3 are N and X1 and X4 are O or S then:R2 and R5 do not exist; R3 and R4 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R3, or R4 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, R12, R13 or R14 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(d) for a second two-nitrogen series, i.e., when X1 and X3 are N and X2 and X4 are O or S then:R3 and R5 do not exist; R2 and R4 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R2, or R4 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, R12, R13 or R14 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(e) for a one-nitrogen series, i.e., when X1 is N and X2, X3 and X4 are O or S then: R3, R4 and R5 do not exist; R2 is independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, R2 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, R12, R13 or R14 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

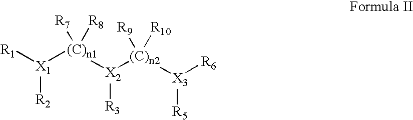

The invention also includes tri-heteroatom acyclic analogues of Formula II where X1, X2, and X3 are independently chosen from the atoms N, S or O such that,

(a) for a three-nitrogen series, when X1, X2, and X3 are N then:R1, R2, R3, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, and n2 are independently chosen to be 2 or 3; and R7, R8, R9, and R10 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R3, R5 or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, or R10 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(b) for a first two-nitrogen series, when X1 and X3 are N and X2 is S or O then:R3 does not exist; R1, R2, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, and n2 are independently chosen to be 2 or 3; and R7, R8, R9, and R10 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R5 or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, or R10 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a second, two-nitrogen series, when X1 and X2 are N and X3 is O or S then: R5 does not exist; R1, R2, R3, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1 and n2 are independently chosen to be 2 or 3; and R7, R8, R9, and R10 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R5, or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, or R10 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

A second series of tri-heteroatom cyclic analogues according to the above Formula II are provided in which R1 and R6 are joined together to form the bridging group (CR11R12)n3, and X1, X2 and X3 are independently chosen from the atoms N, S or O such that:

(a) for a three-nitrogen series, when X1, X2, and X3 are N then:R2, R3, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R2, R3, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(b) for a two-nitrogen series, when X1 and X2 are N and X3 is S or O then:R5 does not exist; R2, and R3 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R2 or R3 may be functionalized for attachment, for example, to peptides, proteins, polyethylene glycols and, other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a one-nitrogen series, when X1 is N and X2 and X3 are O or S then:

R3 and R5 do not exist; R2 is independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, R2 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

The present invention is also directed to treating and preventing neurodegenerative diseases, disorders, and/or conditions in a mammal, including but not limited to the kind referenced herein, and/or enhancing tissue repair processes, including but not limited to neuronal tissue. These include but are not limited to methods for the treatment and prevention for such diseases, disorders, and/or conditions aimed at reduction in available free copper, in particular, Cu⁺². A reduction in extra-cellular copper values, in particular, Cu⁺², is advantageous in that such lower copper levels will lead to a reduction in copper-mediated tissue damage. They can also lead to an improvement in tissue repair by, for example, restoration of normal tissue stem cell responses, and/or solubilsation of amyloid plaques.

In one aspect, the present invention provides a method of treating a subject having or suspected of having or predisposed to a neurodegenerative disease, disorder, and/or condition, comprising administering a pharmaceutically acceptable copper antagonist. Such compounds may be administered in an amount, for example, that is effective to (1) increase copper output in the urine of said subject, or (2) decrease copper uptake in the gastrointestinal tract, or (3) both.

In another aspect the invention provides a method of diminishing copper and/or available copper in a subject having or suspected of having or predisposed to a neurodegenerative disease, disorder, and/or condition comprising administering a pharmaceutically acceptable copper antagonist. Such compounds may be administered in an amount, for example, that is effective to lower copper levels in a subject.

In yet a further aspect the invention provides a method of administering a therapeutically effective amount of a pharmaceutically acceptable copper antagonist formulated in a delayed release preparation, a slow release preparation, an extended release preparation, a controlled release preparation and/or in a repeat action preparation to a subject having or suspected of having or predisposed to a neurodegenerative disease, disorder, and/or condition, including but not limited to those herein disclosed.

In another aspect the invention provides the use of a therapeutically effective amount of a pharmaceutically acceptable copper antagonist in the manufacture of a medicament for the treatment of a subject having or suspected of having or predisposed to a neurodegenerative disease, disorder and/or condition, including but not limited to those herein disclosed.

In another aspect the invention provides the use of a therapeutically effective amount of a copper antagonist in the manufacture of a dosage form for use in the treatment of a subject having or suspected of having or predisposed to a neurodegenerative disease, disorder and/or condition, including but not limited to those herein disclosed.

In a further aspect the invention provides a transdermal patch, pad, wrap or bandage capable of being adhered or otherwise associated with the skin of a subject, said patch being capable of delivering a therapeutically effective amount of a pharmaceutically acceptable copper antagonist to a subject having or suspected of having or predisposed to a neurodegenerative disease, disorder, and/or condition, including but not limited to those herein disclosed.

In another aspect the invention provides an article of manufacture comprising a vessel containing a therapeutically effective amount of a pharmaceutically acceptable copper antagonist and instructions for use for subjects having or suspected of having or predisposed to a neurodegenerative disease, disorder, and/or condition, including but not limited to those herein disclosed.

In another aspect the invention provides an article of manufacture comprising packaging material containing one or more dosage forms containing a pharmaceutically acceptable copper antagonist, wherein the packaging material has a label that indicates that the dosage form can be used for a subject having or suspected of having or predisposed to a neurodegenerative disease, disorder and/or condition, including but not limited to those herein disclosed.

In another aspect the invention provides a formulation comprising a pharmaceutically acceptable copper antagonist that is effective in removing copper from the body of a subject having or suspected of having or predisposed to a neurodegenerative disease, disorder and/or condition, including but not limited to those herein disclosed.

In another aspect the present invention provides a device containing a therapeutically effective amount of a pharmaceutically acceptable copper antagonist comprising a rate-controlling membrane enclosing a drug reservoir employed for the treatment of a subject having or suspected of having or predisposed to having a neurodegenerative disease, disorder, and/or condition, including but not limited to those herein disclosed.

In yet another aspect the invention provides a device containing a pharmaceutically acceptable copper antagonist in a monolithic matrix device employed for the treatment of a subject having or suspected of having or predisposed to a neurodegenerative disease, disorder, and/or condition, including but not limited to those herein disclosed.

Neurodegenerative diseases, disorders, and/or conditions, in which the methods, uses, doses, dose formulations, and routes of administration thereof of the invention will be useful include, for example, dementia, memory impairment caused by dementia, memory impairment seen in senile dementia, various degenerative diseases of the nerves including Alzheimer's disease, Huntingtons disease, Parkinson's disease, parkinsonism, amyotrophic lateral sclerosis (ALS), Friedreich's ataxia and other hereditary ataxia, other diseases, conditions and disorders characterized by loss, damage or dysfunction of neurons including transplantation of neuron cells into individuals to treat individuals suspected of suffering from such diseases, conditions and disorders, any neurodegenerative disease of the eye, including photoreceptor loss in the retina in patients afflicted with macular degeneration, retinitis pigmentosa, glaucoma, and similar diseases, stroke, cerebral ischemia, head trauma, migraine, depression, peripheral neuropathy, pain, cerebral amyloid angiopathy, nootropic or cognition enhancement, multiple sclerosis, ocular angiogenesis, corneal injury, macular degeneration, tumor invasion, tumor growth, tumor metastasis, corneal scarring, scleritis, motor neuron and Lewy body disease, attention deficit disorder, narcolepsy, psychiatric disorders, panic disorders, social phobias, anxiety, psychoses, obsessive-compulsive disorders, obesity or eating disorders, body dysmorphic disorders, post-traumatic stress disorders, conditions associated with aggression, drug abuse treatment, or smoking secession, traumatic brain and spinal cord injury, and epilepsy.

In one embodiment the neurodegenerative disease is Alzheimer's disease. In another embodiment the neurodegenerative disease is Parkinson's disease

Copper antagonists useful in the prevention or treatment of one or more of the diseases described or listed herein include, but are not limited to, those compounds set forth in Formula I and Formula II.

In another embodiment the copper antagonist is a triene that chelates copper. Copper antagonists also include, but are not limited to, trientine, including trientine acid addition salts and active metabolites including, for example, N-acetyl trientine, and analogues, derivatives, and prodrugs thereof. In one embodiment, the trientine is rendered less basic (e.g., as an acid addition salt).

Salts of trientine (which optionally can be salts of a prodrug of trientine or a copper chelating metabolite of trientine) include, in one embodiment, acid addition salts such as, for example, those of suitable mineral or organic acids. Salts of trientine (such as acid addition salts, e.g., trientine hydrochloride, trientine dihydrochloride, trientine trihydrochloride, and trientine tetrahydrochloride) act as copper-chelating agents that aid in the elimination of copper from the body by forming a stable soluble complex that is readily excreted by the kidney. Trientine succinate salts are also preferred.

In another embodiment, the copper antagonist, for example a trientine, is modified. For example, it may be as an analogue or derivative, for example an analogue or derivative of trientine (or an analogue or derivative of a copper-chelating metabolite of trientine, for example, N-acetyl trientine).

Derivatives of copper antagonists, including trientine or trientine salts or analogues, include those modified with polyethylene glycol (PEG). The structure of PEG is HO—(—CH₂—CH₂—O—)ₙ—H. It is a linear or branched, neutral polyether available in a variety of molecular weights.

Copper antagonists analogues include, for example, compounds in which one or more sulfur molecules are substituted for one or more of the NH groups. Other analogues include, for example, compounds in which trientine has been modified to include one or more additional —CH₂ groups.

Analogues of trientine include, for example, compounds in which one or more sulfur molecules is substituted for one or more of the NH groups in trientine. Other analogues include, for example, compounds in which trientine has been modified to include one or more additional —CH₂ groups. The chemical formula of trientine is NH₂—CH₂—CH₂—NH—CH₂—CH₂—NH—CH₂—CH₂—NH₂. The empirical formula is C₆N₄H₁₈. Analogues of trientine include, for example:
1. SH—CH₂—CH₂—NH—CH₂—CH₂—NH—CH₂—CH₂—NH₂,
2. SH—CH₂—CH₂—S—CH₂—CH₂—NH—CH₂—CH₂—NH₂,
3. NH2—CH2-CH2—NH—CH2—CH2-S—CH2—CH2-SH,
4. NH₂—CH₂—CH₂—S—CH₂—CH₂—S—CH₂—CH₂—SH,
5. SH—CH₂—CH₂—S—CH₂—CH₂—S—CH₂—CH₂—SH,
6. NH₂—CH₂—CH₂—NH—CH₂—CH₂—CH₂—NH—CH₂—CH₂—NH₂,
7. SH—CH₂—CH₂—NH—CH₂—CH₂—CH₂—NH—CH₂—CH₂—NH₂,
8. SH—CH₂—CH₂—S—CH₂—CH₂—CH₂—NH—CH₂—CH₂—NH₂,
9. NH₂—CH₂—CH₂—NH—CH₂—CH₂—CH₂—S—CH₂—CH₂—SH,
10. NH₂—CH₂—CH₂—S—CH₂—CH₂—CH₂—S—CH₂—CH₂—SH,
11. SH—CH₂—CH₂—S—CH₂—CH₂—CH₂—S—CH₂—CH₂—SH,
12. and so on.

One or more hydroxyl groups may also be substituted for one or more amine groups to create a copper antagonist analogue.

One or more hydroxyl groups may also be substituted for one or more amine groups to create an analogue of trientine (with or without the substitution of one or more sulfurs for one or more nitrogens).

In another embodiment, a copper antagonist is trientine is delivered as a prodrug of trientine or a copper chelating metabolite of trientine.

In another embodiment the copper antagonist is a trientine active agent. Trientine active agents include, for example, trientine, salt(s) of trientine, a trientine prodrug or a salt of such a prodrug, a trientine analogue or a salt or prodrug of such an analogue, and/or at least one active metabolite of trientine or a salt or prodrug of such a metabolite, including but not limited to N-acetyl trientine and salts and prodrugs of N-acetyl trientine. Trientine active agents also include the analogues of Formulae I and II and/or prodrugs and/or salts of said prodrugs of said analogues.

In another embodiment the dosage form and/or therapeutically effective amount is able to provide an effective daily dosage to the subject of a copper chelator of about 4 g per day or below although if given orally the dosage is generally from about 1 mg to about 4 g per day. In another embodiment the oral dose delivery (cumulative or otherwise) is in the range of from 200 mg to 4 g per day if given orally. In a further embodiment the daily dosage is such as to deliver about 600 mg to about 1.2 g per day.

In another embodiment the effective amount administered is from about 5 mg to about 2400 mg per dose and/or per day. Other effective dose ranges of copper antagonists, for example, compounds of Formulae I and II, and trientine active agents, including but not limited to trientine, trientine salts, trientine analogues of, and so on, for example, include from 10 mg to 1100 mg, 10 mg to 1000 mg, 10 mg to 900 mg, 20 mg to 800 mg, 30 mg to 700 mg, 40 mg to 600 mg, 50 mg to 500 mg, 50 mg to 450 mg, from 50-100 mg to about 400 mg, 50-100 mg to about 300 mg, 110 to 290 mg, 120 to 280 mg, 130 to 270 mg, 140 to 260 mg, 150 to 250 mg, 160 to 240 mg, 170 to 230 mg, 180 to 220 mg, 190 to 210 mg, and/or any other amount within the ranges as set forth.

In a further embodiment the copper antagonist may be administered orally as for example, an oral composition. Examples of suitable oral compositions of the invention include, but are not limited to, tablets, capsules, lozenges, or like forms, or any liquid forms such as syrups, aqueous solutions, emulsions and the like.

In a further embodiment the copper antagonist may be administered parenterally, for example, as a parenteral composition. The parenteral composition may include, depending on the rate of parenteral administration, for example, solutions, suspensions, emulsions that can be administered by subcutaneous, intravenous, intramuscular, intradermal, intrasternal injection or infusion techniques. In one embodiment, the parenteral formulation is capable, for example, of maintaining constant plasma concentrations of the copper antagonist for extended periods. The parenteral composition can further include, for example, any one or more of the following a buffer, for example, an acetate, phosphate, citrate or glutamate buffer to obtain a pH of the final formulation from approximately 5.0 to 9.5, a carbohydrate or polyhydric alcohol tonicifier, an antimicrobial preservative that may be selected from the group of, for example, m-cresol, benzyl alcohol, methyl, ethyl, propyl and butyl parabens and phenol and a stabilizer. A sufficient amount of water for injection is used to obtain the desired concentration of the parenteral composition. Sodium chloride, as well as other excipients, may also be present, if desired. Such excipients, however, must maintain the overall stability of the copper antagonist. The parenteral composition should generally be substantially isotonic. An isotonic solution may be defined as a solution that has a concentration of electrolytes, non-electrolytes, or a combination of the two that will exert an equivalent osmotic pressure as that into which it is being introduced, in this case, mammalian tissue. By “substantially isotonic” is meant within ±20% of isotonicity, preferably within ±10%. The parenteral composition may be included within a container, typically, for example, a vial, cartridge, prefilled syringe or disposable pen.

In another embodiment the copper antagonist may be delivered transdermally. Examples of compositions or dosage forms suitable for transdermal administration include transdermal patches, transdermal bandages, and the like.

In another embodiment the copper antagonist may be administered topically. Examples of compositions or dosage forms suitable for topical administration include but are not limited to lotions, sticks, sprays, ointments, pastes, creams, gels, and the like, whether applied directly to the skin or via an intermediary such as a pad, patch or the like.

In a further embodiment the copper antagonists of the invention may be administered by suppositories, as for example, any solid dosage form inserted into a bodily orifice particularly those, for example, inserted rectally, vaginally, and/or urethrally.

In another embodiment the copper antagonist of the invention may be administered transmucosolly. Examples of compositions and/or dosage forms suitable for transmuscosal administration include but are not limited to solutions for enemers, pessaries, tampons, creams, gels, pastes, foams, nebulised solutions, powders, in similar formulations.

In another embodiment the copper antagonists of the invention are administered by depot administration. Examples of compositions and/or dosage forms suitable for depot administration include, but are not limited to, pellets or small cyclinders of copper antagonist or solid forms wherein the copper antagonist is entrapped in a matrix of biodegradable polymers, micro emulsions, liposomes and/or is microencapsulated.

In a further embodiment, the copper antagonist of the invention is administered by way of infusion devices, including but not limited to, implantable infusion devices and infusion pumps including implantable infusion pumps.

In a further embodiment, the copper antagonist of the invention may be administered by inhalation or insufflation. Examples of composition and/or dosage forms suitable for administration by inhalation or insufflation include, but are not limited to, solutions and/or suspensions in pharmaceutically acceptable, aqueous, or organic solvents, or mixtures thereof and/or powders.

In a further embodiment the copper antagonists of the invention may be administered by buccal or sublingual administration. Examples of compositions and/or dosage forms suitable for administration by buccal or sublingual administration include, but are not limited to, lozenges, tablets, capsules, and the like, and/or compositions comprising solutions and/or suspensions in pharmaceutically acceptable, aqueous, or organic solvents, or mixtures thereof and/or powders.

In a further embodiment the copper antagonist of the invention may be administered by way of opthalmic administration. Examples of compositions and/or dosage forms suitable for opthalmic administration include compositions comprising solutions and/or suspensions of the copper chelator of the invention in pharmaceutically acceptable, aqueous or organic solvents, and/or inserts.

In another embodiment the monolithic matrix device contains a copper antagonist in a dispersed soluble matrix, in which the copper antagonist becomes increasingly available as the matrix dissolves or swells. The monolithic matrix device, may include, but is not limited to, one or more of the following excipients:hydroxypropylcellulose (BP) or hydroxypropyl cellulose (USP); hydroxypropyl methylcellulose (BP, USP); methylcellulose (BP, USP); calcium carboxymethylcellulose (BP, USP); acrylic acid polymer or carboxy polymethylene (Carbopol) or Carbomer (BP, USP); or linear glycuronan polymers such as alginic acid (BP, USP), for example those formulated into microparticles from alginic acid (alginate)-gelatin hydrocolloid coacervate systems, or those in which liposomes have been encapsulated by coatings of alginic acid with poly-L-lysine membranes. Alternatively, said monolithic matrix includes the copper antagonist dissolved in an insoluble matrix and becomes available as an aqueous solvent enters the matrix through micro-channels and dissolves the copper antagonist particles.

In a further embodiment the monolithic matrix contains the copper antagonist, for example, as particles in a lipid matrix or insoluble polymer matrix, including, but not limited to, preparations formed from Carnauba wax (BP; USP); medium-chain triglyceride such as fractionated coconut oil (BP) or triglycerida saturata media (PhEur); or cellulose ethyl ether or ethylcellulose (BP, USP). The lipids can be present in said monolithic matrix from between 20-40% hydrophobic solids w/w. The lipids may remain intact during the release process.

In another embodiment the device may contain in addition to the copper antagonist, one or more of the following, for example, a channeling agent, such as sodium chloride or one or more sugars, which leaches from the formulation, forming aqueous micro-channels (capillaries) through which solvent enters, and through which drug is released.

Alternatively, the device is any hydrophilic polymer matrix, in which said copper antagonist is compressed as a mixture with any water-swellable hydrophilic polymer.

In one embodiment the hydrophilic polymer matrix contains in addition to a copper antagonist any one or more of the following, for example, a gel modifier such as one or more of a sugar, counter ions, a pH buffer, a surfactant, a lubricant such as a magnesium stearate and/or a glidant such as colloidal silicon dioxide.

Copper antagonist compounds within Formula I and Formula II may also be used in the prevention or treatment of one or more other diseases, disorders, and/or conditions that would benefit from copper removal, particularly removal of Cu⁺². Such diseases, disorders, and/or conditions include but are not limited to heart failure, coronary artery disease, cardiomyopathy, myocardial infarction, obesity, Syndrome X, insulin resistance, diabetes, diabetic complications (including, for example, but not limited to, neuropathy, nephropathy, retinopathy, myopathy, dermopathy, diabetic cardiomyopathy, coronary artery disease, macroangiopathy, microangiopathy, and peripheral vascular disease), diabetic acute coronary syndrome (e.g., myocardial infarction), diabetic hypertensive cardiomyopathy, acute coronary syndrome associated with impaired glucose tolerance (IGT), acute coronary syndrome associated with impaired fasting glucose (IFG), hypertensive cardiomyopathy associated with IGT, hypertensive cardiomyopathy associated with IFG, ischaemic cardiomyopathy associated with IGT, ischaemic cardiomyopathy associated with IFG, myocardial infarction (AMI) associated with impaired glucose tolerance (IGT), myocardial infarction associated with impaired fasting glucose (IFG), ischaemic cardiomyopathy associated with coronary heart disease (CHD), myocardial infarction not associated with any abnormality of the glucose metabolism, acute coronary syndrome not associated with any abnormality of the glucose metabolism, hypertensive cardiomyopathy not associated with any abnormality of the glucose metabolism, ischaemic cardiomyopathy not associated with any abnormality of the glucose metabolism (irrespective of whether or not such ischaemic cardiomyopathy is associated with coronary heart disease or not), and any disease of the vascular tree including disease states of the aorta, carotid, cerebrovascular, coronary, renal, retinal, vasa nervorum, iliac, femoral, popliteal, arteriolar tree and capillary bed.

### BRIEF DESCRIPTION OF THE DRAWINGS

FIG. 1 shows the urine excretion in diabetic and non-diabetic animals in response to increasing doses of the copper antagonist trientine or equivalent volume of saline, wherein urine excretion in diabetic and nondiabetic animals in response to increasing doses of trientine (bottom; 0.1, 1.0, 10, 100 mg.kg⁻¹ in 75 μl saline followed by 125 μl saline flush injected at time shown by arrow) or an equivalent volume of saline (top), and each point represents a 15 min urine collection period (see Example 2 Methods for details); error bars show SEM and P values are stated if significant (P<0.05).

FIG. 2 shows urine excretion in non-diabetic and diabetic animals receiving increasing doses of trientine or an equivalent volume of saline, wherein urine excretion in diabetic (top) and nondiabetic (bottom) rats receiving increasing doses of trientine (0.1, 1.0, 10, 100 mg.kg⁻¹ in 75 μl saline followed by 125 μl saline flush injected at time shown by arrow) or an equivalent volume of saline, and each point represents a 15 min urine collection period (see Example 2 Methods for details); error bars show SEM and P values are stated if significant (P<0.05).

FIG. 3 shows copper excretion in the urine of diabetic and non-diabetic animals receiving increasing doses of trientine or an equivalent volume of saline, wherein copper excretion in urine of diabetic (top) and nondiabetic (bottom) rats receiving increasing doses of trientine (0.1, 1.0, 10, 100 mg.kg⁻¹ in 75 μl saline followed by 125 μl saline flush injected at time shown by arrow) or an equivalent volume of saline, and each point represents a 15 min urine collection period (see Example 2 Methods for details); error bars show SEM and P values are stated if significant (P<0.05).

FIG. 4 shows the same information in FIG. 3 with presentation of urinary copper excretion per gram of bodyweight, wherein urinary copper excretion per gram of bodyweight in diabetic and nondiabetic animals in response to increasing doses of trientine (bottom; 0.1, 1.0, 10, 100 mg.kg⁻¹ in 75 μl saline followed by 125 μl saline flush injected at time shown by arrow) or an equivalent volume of saline (top), and each point represents a 15 min urine collection period (see Example 2 Methods for details); error bars show SEM and P values are stated if significant (P<0.05).

FIG. 5 shows the total amount of copper excreted in non-diabetic and diabetic animals administered saline or drug, wherein total urinary copper excretion (μmol) in nondiabetic animals administered saline (black bar, n=7) or trientine (hatched bar, n=7) and in diabetic animals administered saline (grey bar, n=7) or trientine (white bar, n=7); error bars show SEM and P values are stated if significant (P<0.05).

FIG. 6 shows the total amount of copper excreted per gram of bodyweight in animals receiving trientine or saline, wherein total urinary copper excretion per gram of bodyweight (μmol.gBW⁻¹) in animals receiving trientine (nondiabetic:hatched bar, n=7; diabetic:white bar, n=7) or saline (nondiabetic:black bar, n=7; diabetic:grey bar, n=7); error bars show SEM and P values are stated if significant (P<0.05).

FIG. 7 shows the iron excretion in urine of diabetic and non-diabetic animals receiving increasing doses of trientine or an equivalent volume of saline, wherein iron excretion in urine of diabetic (top) and nondiabetic (bottom) rats receiving increasing doses of trientine (0.1, 1.0, 10, 100 mg.kg⁻¹ in 75 μl saline followed by 125 μl saline flush injected at time shown by arrow) or an equivalent volume of saline, and each point represents a 15 min urine collection period (see Example 2 Methods for details); error bars show SEM and P values are stated if significant (P<0.05).

FIG. 8 shows the urinary iron excretion per gram of bodyweight in diabetic and non-diabetic animals receiving trientine or saline, wherein urinary iron excretion per gram of bodyweight in diabetic and nondiabetic animals in response to increasing doses of trientine (bottom; 0.1, 1.0, 10, 100 mg.kg⁻¹ in 75 μl saline followed by 125 μl saline flush injected at time shown by arrow) or an equivalent volume of saline (top), and each point represents a 15 min urine collection period (see Example 2 Methods for details); error bars show SEM and P values are stated if significant (P<0.05).

FIG. 9 shows the total urinary iron excretion in non-diabetic and diabetic animals administered saline or drug, wherein total urinary iron excretion (μmol) in nondiabetic animals administered saline (black bar, n=7) or trientine (hatched bar, n=7) and in diabetic animals administered saline (grey bar, n=7) or trientine (white bar, n=7); error bars show SEM and P values are stated if significant (P<0.05).

FIG. 10 shows the total urinary iron excretion per gram of bodyweight in animals receiving trientine or saline, wherein total urinary iron excretion per gram of bodyweight (μmol.gBW⁻¹) in animals receiving trientine (nondiabetic:hatched bar, n=7; diabetic:white bar, n=7) or saline (nondiabetic:black bar, n=7; diabetic:gray bar, n=7); error bars show SEM and P values are stated if significant (P≦0.05).

FIG. 11 shows urinary [Cu] by AAS (Δ) and EPR (▴) following sequential 10 mg.kg⁻¹ (A) and 100 (B) trientine boluses; (inset) background-corrected EPR signal from 75-min urine indicating presence of Cu^{II}-trientine; *, P<0.05, **, P<0.01 vs. control.

FIG. 12 is a table comparing the copper and iron excretion in the animals receiving trientine or saline, which is a statistical analysis using a mixed linear model.

FIG. 13 shows the body weight of animals changing over the time period of experiment in Example 5.

FIG. 14 shows the glucose levels of animals changing over the time period of the experiment in Example 5.

FIG. 15 is a diagram showing cardiac output in animals as measured in Example 5.

FIG. 16 is a diagram showing coronary flow in animals as measured in Example 5.

FIG. 17 is a diagram showing coronary flows normalized to final cardiac weight in animals as measured in Example 5.

FIG. 18 is a diagram showing aortic flow in animals as measured in Example 5.

FIG. 19 is a diagram showing the maximum rate of positive change in pressure development in the ventricle with each cardiac cycle (contraction) in animals as measured in Example 5.

FIG. 20 is a diagram showing the maximum rate of decrease in pressure in the ventricle with each cardiac cycle (relaxation) in animals as measured in Example 5.

FIG. 21 shows the percentage of functional surviving hearts at each after-load in animals as measured in Example 5.

FIG. 22 shows the structure of LV-myocardium from STZ-diabetic and matched non-diabetic control rats following 7-w oral trientine treatment, wherein cardiac sections were cut following functional studies. Each image is representative of 5 independent sections per heart×3 hearts per treatment. a-d, Laser confocal images of 120-μM LV sections co-stained for actin (Phalloidin-488, orange) and immunostained for β₁-integrin (CY5-conjugated secondary antibody, purple) (scale-bar=33 μm). a, Untreated-control; b, Untreated-diabetic; c, Trientine treated diabetic; d, Trientine-treated non-diabetic control. e-h, TEM images of corresponding 70-nM sections stained with uranyl acetate/lead citrate (scale-bar=158 nm); e, Untreated-control; f, Untreated-diabetic; g, Trientine-treated diabetic; h, Trientine-treated non-diabetic control.

FIG. 23 shows effect of 6 months' oral trientine treatment on LV mass in humans with T2DM, wherein trientine (600 mg twice-daily) or matched placebo were administered to subjects with diabetes (n=15) or matched controls (n=15) in a double-blind, parallel-group study, and wherein differences in LV mass (g; mean and 95% confidence interval) were determined by tagged-cardiac MRI.

FIG. 24 shows a randomized, double blind, placebo-controlled trial comparing effects of oral trientine and placebo on urinary Cu excretion from male humans with uncomplicated T2DM and matched non-diabetic controls, wherein urinary Cu excretion (μmol.2 h⁻¹ on day 1 (baseline) and day 7 following a single 2.4-g oral dose of trientine or matched placebo to subjects described in Table 9, placebo-treated T2DM, ∘, placebo-treated control, ●, trientine-treated T2DM, □; trientine treated control, ▪. Cu excretion from T2DM following trientine-treatment was significantly greater than that from trientine-treated non-diabetic controls (P<0.05).

FIG. 25 shows mean arterial pressure (MAP) response in diabetic and nondiabetic animals to 10 mg.kg⁻¹ Trientine in 75 μl+125 μl saline flush (or an equivalent volume of saline). Each point represents one minute averages of data points collected every 2 seconds. The time of drug (or saline) administration is indicated by the arrow. Error bars show SEM,

FIG. 26 shows the ultraviolet-visible spectral trace of the trientine containing formulation after being stored for 15 days and upon the addition of copper to form the trientine-copper complex. The traces were taken on day 0 (control formulation) and day 15. There were three formulations containing trientine one was stored in the dark at 4° C., the second at room temperature (21° C.) in the dark and a third at room temperature in daylight. When the spectral was taken copper was added, and

FIG. 27 shows neurons and astrocytes that had been grown for two weeks in growth media containing foetal bovinve serum, fixed with neutral buffered formalin and then stained with anti-BSA antibodies (green). The arrows point towards the internalized BSA in the neurons and astrocytes. A, E:show diffuse staining of the whole cell body along with discrete units of stain in small “balloon-like” structures. B, C:are neuronal cells stained for the presence of BSA. D:shows the neuronal cells from C double stained with anti-Neu (cyan colour). Omission of the primary anti-Bovine Serum Albumin antibody in the control eliminated staining. Scale bar A, B, D,=15 μm, C,D, control=30 μm.

### DETAILED DESCRIPTION

As used herein, a “copper antagonist” is a pharmaceutially acceptable compound that binds or chelates copper, preferably copper (II), in vivo for removal. Copper chelators are presently preferred copper antagonists. Copper (II) chelators, and copper (II) specific chelators (i.e., those that preferentially bind copper (II) over other forms of copper such as copper (I)), are especially preferred. “Copper (II)” refers to the oxidized (or +2) form of copper, also sometimes referred to as Cu⁺².

As used herein, a “disorder” is any disorder, disease, or condition that would benefit from an agent that reduces local or systemic copper or copper concentrations. Particularly preferred are agents that reduce extracellular copper or extracellular copper concentrations (local or systemic) and, more particularly, agents that reduce extracellular copper (II) or extracellular copper (II) concentrations (local or systemic). Disorders include, but are not limited to, tissue damage and vascular damage.

As used herein, “mammal” refers to any animal classified as a mammal, including humans, domestic and farm animals, and zoo, sports, or pet animals, such as dogs, horses, cats, sheep, pigs, cows, etc. The preferred mammal herein is a human.

As used herein, “pharmaceutically acceptable salts” refers to salts prepared from pharmaceutically acceptable non-toxic bases or acids including inorganic or organic bases and inorganic or organic acids the like. When the copper antagonist compound is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are hydrochloric and succinic acids.

As used herein, “preventing” means preventing in whole or in part, or ameliorating or controlling.

As used herein, a “therapeutically- or pharmaceutically-effective amount” in reference to the compounds or compositions of the instant invention refers to the amount sufficient to induce a desired biological result. That result can be alleviation of the signs, symptoms, or causes of a disease or disorder or condition, or any other desired alteration of a biological system. In the present invention, the result will typically involve the prevention, decrease, or reversal of tissue injury, in whole or in part.

As used herein, the term “treating” refers to both therapeutic treatment and prophylactic or preventative measures. Those in need of treatment include those already with the disorder as well as those prone to having the disorder or diagnosed with the disorder or those in which the disorder is to be prevented.

A reduction in copper, particularly extracellular copper that is generally in the its copper II form, will be advantageous in the treatment of neurodegenerative disorders, diseases, and/or conditions, caused or exacerbated by mechanisms that may be affected by or are dependent on excess copper values. For example, a reduction in copper will be advantageous in providing a reduction in and/or reversal of copper associated damage. It will also be advantageous in providing improved tissue repair by restoration of normal tissue stem cell responses, and/or by a decrease in copper-mediated insolubility of plaque forming polypeptides such as, for example but not limited to, Aβ, and/or a reduction in copper-mediated neurofibrillary tangle formation.

Wilson's disease is due to an inherited defect in copper excretion into the bile by the liver. The resulting copper accumulation and copper toxicity primarily results in liver disease. Patients generally present, between the ages of 10 and 40 years. Wilson's disease is effectively treated with orally administered copper chelators. It has been demonstrated that chelated copper in patients with Wilson's disease is excreted primarily through the feces, either by the effective chelation of copper in the gut (or inhibition of absorption), or by partial restoration of mechanisms that allow for excretion of excess copper via urine or into the bile, or a combination of the two. See Siegemund R, et al., “Mode of action of triethylenetetramine dihydrochloride on copper metabolism in Wilson's disease,” Acta Neurol Scand. 83(6):364-6 (June 1991).

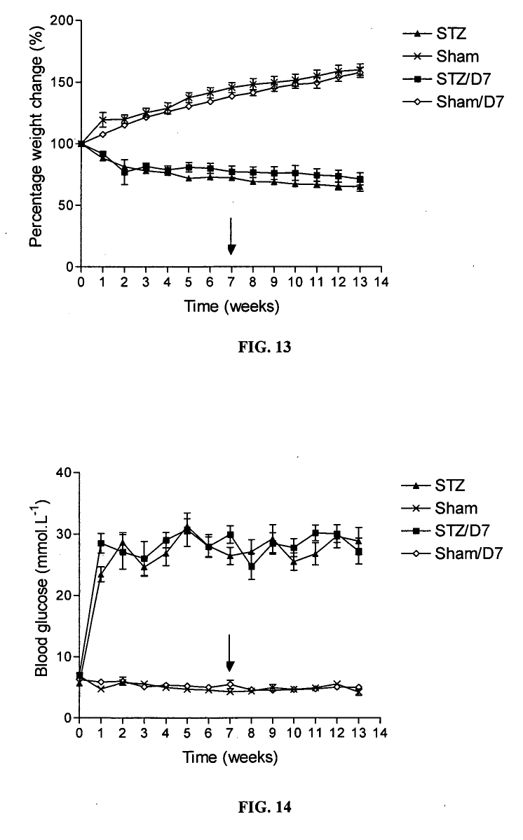

In contrast, experiments described herein unexpectedly revealed that administration of the copper chelator trientine dihydrochloride, for example, to non-Wilson's disease patients does not result in increased excretion of copper in the feces. See Example 6 and Table 4. Rather, excretion of excess copper in non-Wilson's disease patients treated with copper chelators occurs primarily, if not virtually exclusively, through the urine rather than the feces. See Example 5 and FIG. 13. These data support the idea that systemic (parenteral) administration of doses of copper antagonists including those doses that are lower than those given orally, or controlled release administration of doses of copper antagonists including those doses that are lower than those given orally, or oral administration of dose forms that avoid undesired first pass clearance such that more active ingredient is available for its intended purpose outside the gut, will be of significant benefit in the indications described herein, for example. This includes methods and uses and/or administration of doses and dose forms that utilize and/or provide for metered release directly into the circulatory system (including intramuscular, intraperitoneal, subcutaneous and intravenous administration) rather than indirectly through the gut. Thus, compositions of the invention may also be formulated for parenteral injection (including, for example, by bolus injection or continuous infusion) and may be presented in unit dose form in ampules, pre-filled syringes, small bolus infusion containers, or in multi-does containers with an added preservative.

According to the invention, methods, uses, compositions and/or doses and dose formulations of copper antagonists, including for example, a compound of Forumlae I or II, or a trientine active agent, that helps to maintain desired blood and tissue levels may be prepared that are highly effective in causing removal of systemic copper from the body via the urine, and may do so at lower doses than required for oral administration given that gut copper need not be excreted, and will be more effective in the treatment of any neurodegenerative disease, disorder, and/or condition, in which pathologically increased or undesired tissue copper plays a role in disease initiation or progression.

Trientine is a strongly basic moiety with multiple nitrogens that can be converted into a large number of suitable associated acid addition salts using an acid, for example, by reaction of stoichiometrically equivalent amounts of trientine and of the acid in an inert solvent such as ethanol or water and subsequent evaporation if the dosage form is best formulated from a dry salt. Possible acids for this reaction are in particular those that yield physiologically acceptable salts. Nitrogen-containing copper antagonists, for example, trientine active agents such as, for example, trientine, that can be delivered as a salt(s) (such as acid addition salts, e.g., trientine dihydrochloride) act as copper-chelating agents or antagonists, which aids the elimination of copper from the body by forming a stable soluble complex that is readily excreted by the kidney. Thus inorganic acids can be used, e.g., sulfuric acid, nitric acid, hydrohalic acids such as hydrochloric acid or hydrobromic acid, phosphoric acids such as orthophosphoric acid, sulfamic acid. This is not an exhaustive list. Other organic acids can be used to prepare suitable salt forms, in particular aliphatic, alicyclic, araliphatic, aromatic or heterocyclic mono- or polybasic carboxylic, sulfonic or sulfuric acids, (e.g., formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono-and-disulfonic acids, and laurylsulfuric acid). Those in the art will be able to prepare other suitable salt forms. Nitrogen-containing copper antagonists, for example, trientine active agents such as, for example, trientine, can also be in the form of quarternary ammonium salts in which the nitrogen atom carries a suitable organic group such as an alkyl, alkenyl, alkynyl or aralkyl moiety. In one embodiment such nitrogen-containing copper antagonists are in the form of a compound or buffered in solution and/or suspension to a near neutral pH much lower than the pH 14 of a solution of trientine itself.

Other trientine active agents include derivative trientine active agents, for example, trientine in combination with picolinic acid (2-pyridinecarboxylic acid). These derivatives include, for example, trientine picolinate and salts of trientine picolinate, for example, trientine picolinate HCl. These also include, for example, trientine di-picolinate and salts of trientine di-picolinate, for example, trientine di-picolinate HCl. Picolinic acid moieties may be attached to trientine, for example one or more of the CH₂ moieties, using chemical techniques known in the art. Those in the art will be able to prepare other suitable derivatives, for example, trientine-PEG derivatives, which may be useful for particular dosage forms including oral dosage forms having increased bioavailablity.

Other copper antagonists include cyclic and acyclic compounds according to the following formulae, for example: 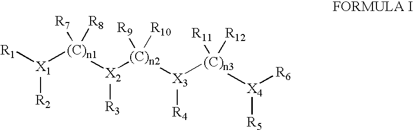
Tetra-heteroatom acyclic compounds within Formula I are provided where X1, X2, X3, and X4 are independently chosen from the atoms N, S or O, such that,

(a) for a four-nitrogen series, i.e., when X1, X2, X3, and X4 are N then:R1, R2, R3, R4, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R3, R4, R5, or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(b) for a first three-nitrogen series, i.e., when X1, X2, X3, are N and X4 is S or O then:R6 does not exist; R1, R2, R3, R4 and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R3, R4, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a second three-nitrogen series, i.e., when X1, X2, and X4 are N and X3 is O or S then:R4 does not exist and R1, R2, R3, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R3, R5, or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(d) for a first two-nitrogen series, i.e., when X2 and X3 are N and X1 and X4 are O or S then:R1 and R6 do not exist; R2, R3, R4, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R2, R3, R4, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(e) for a second two-nitrogen series, i.e., when X1 and X3 are N and X2 and X4 are O or S then:R3 and R6 do not exist; R1, R2, R4, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R4, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(f) for a third two-nitrogen series, i.e., when X1, and X2 are N and X3 and X4 are O or S then:R4 and R6 do not exist; R1, R2, R3, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R3, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(g) for a fourth two-nitrogen series, i.e., when X1 and X4 are N and X2 and X3 are O or S then:R3 and R4 do not exist; R1, R2, R5 and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R5, or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Second, for a tetra-heteroatom series of cyclic analogues, R1 and R6 are joined together to form the bridging group (CR13R14)n4, and X1, X2, X3, and X4 are independently chosen from the atoms N, S or O such that,

(a) for a four-nitrogen series, i.e., when X1, X2, X3, and X4 are N then:R2, R3, R4, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R2, R3, R4, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R7, R8, R9, R10, R11, R12, R13 or R14 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(b) for a three-nitrogen series, i.e., when X1, X2, X3, are N and X4 is S or O then: R5 does nor exist; R2, R3, and R4 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R2, R3 or R4 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, R12, R13 or R14 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a first two-nitrogen series, i.e., when X2 and X3 are N and X1 and X4 are O or S then:R2 and R5 do not exist; R3 and R4 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R3, or R4 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, R12, R13 or R14 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(d) for a second two-nitrogen series, i.e., when X1 and X3 are N and X2 and X4 are O or S then:R3 and R5 do not exist; R2 and R4 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R2, or R4 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, R12, R13 or R14 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(e) for a one-nitrogen series, i.e., when X1 is N and X2, X3 and X4 are O or S then:R3, R4 and R5 do not exist; R2 is independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, R2 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, R12, R13 or R14 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. 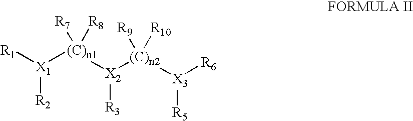
Tri-heteroatom compounds within Formula II are provided where X1, X2, and X3 are independently chosen from the atoms N, S or O such that,

(a) for a three-nitrogen series, when X1, X2, and X3 are N then:R1, R2, R3, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, and n2 are independently chosen to be 2 or 3; and R7, R8, R9, and R10 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R3, R5 or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, or R10 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(b) for a first two-nitrogen series, when X1 and X3 are N and X2 is S or O then: R3 does not exist; R1, R2, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, and n2 are independently chosen to be 2 or 3; and R7, R8, R9, and R10 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R5 or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, or R10 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a second, two-nitrogen series, when X1 and X2 are N and X3 is O or S then:R5 does not exist; R1, R2, R3, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1 and n2 are independently chosen to be 2 or 3; and R7, R8, R9, and R10 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R1, R2, R5, or R6 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, or R10 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

A second series of tri-heteroatom cyclic analogues according to the above Formula II are provided in which R1 and R6 are joined together to form the bridging group (CR11R12)n3, and X1, X2 and X3 are independently chosen from the atoms N, S or O such that:

(a) for a three-nitrogen series, when X1, X2, and X3 are N then:R2, R3, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R2, R3, or R5 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(b) for a two-nitrogen series, when X1 and X2 are N and X3 is S or O then:R5 does not exist; R2, and R3 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R2 or R3 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a one-nitrogen series, when X1 is N and X2 and X3 are O or S then:

R3 and R5 do not exist; R2 is independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, R2 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R7, R8, R9, R10, R11, or R12 may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

The compounds of the invention, including trientine active agents, may be made using any of a variety of chemical synthesis, isolation, and purification methods known in the art. Exemplary synthetic routes are described below.

General synthetic chemistry protocols are somewhat different for these classes of molecules due to their propensity to chelate with metallic cations, including copper. Glassware should be cleaned and silanized prior to use. Plasticware should be chosen specifically to have minimal presence of metal ions. Metal implements such as spatulas should be excluded from any chemistry protocol involving chelators. Water used should be purified by sequential carbon filtering, ion exchange and reverse osmosis to the highest level of purity possible, not by distillation. All organic solvents used should be rigorously purified to exclude any possible traces of metal ion contamination.

Care must also be take with purification of such derivatives due to their propensity to chelate with a variety of cations, including copper, which may be present in trace amounts in water, on the surface of glass or plastic vessels. Once again, glassware should be cleaned and silanized prior to use. Plasticware should be chosen specifically to have minimal presence of metal ions. Metal implements such as spatulas should be avoided, and water used should be purified by sequential carbon filtering, ion exchange and reverse osmosis to the highest level of purity possible, and not by distillation. All organic solvents used should be rigorously purified to exclude any possible traces of metal ion contamination. Ion exchange chromatography followed by lyophilization is typically the best way to obtain pure solid materials of these classes of molecules. Ion exchange resins should be washed clean of any possible metal contamination.

Acyclic and cyclic compounds of the invention and exemplary synthetic methods and existing syntheses from the art include the following:

For Tetra-Heteroatom Acyclic Examples of Formula I:

X1, X2, X3, and X4 are independently chosen from the atoms N, S or O such that:

4N Series:
when X1, X2, X3, and X4 are N then:
R1, R2, R3, R4, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, and n3 are independently chosen to be 2 or 3, and each repeat of any of n1, n2, and n3 may be the same as or different than any other repeat; and
R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R1, R2, R3, R4, R5, or R6 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, R10, R11, or R12 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Also provided are embodiments wherein one, two, three or four of R1 through R12 are other than hydrogen.

In some embodiments, the compounds of Formula I or II are selective for a particular oxidation state of copper. For example, the compounds may be selected so that they preferentially bind oxidized copper, or copper (II). Copper selectivity can be assayed using methods known in the art. Competition assays can be done using isotopes of copper (I) and copper (II) to determine the ability of the compounds to selectively bind one form of copper.

In some embodiments, the compounds of Formula I or II may be chosen to avoid excessive lipophilicity, for example by avoiding large or numerous alkyl substituents. Excessive lipophilicity can cause the compounds to bind to and/or pass through cellular membranes, thereby decreasing the amount of compound available for chelating copper, particularly for extracellular copper, which may be predominantly in the oxidized form of copper (II).

### Synthesis of Examples of the Open Chain 4N Series of Formula I

Trientine itself has been synthesized by reaction of 2 equivalents of ethylene diamine with 1,2-dichloro ethane to give trientine directly (1). Modification of this procedure by using starting materials with appropriate R groups would lead to symmetrically substituted open chain 4N examples as shown below:

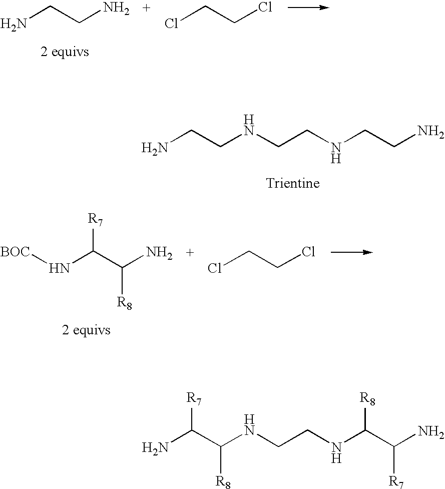

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the tetra-aza series. In order to obtain the un-symmetrically substituted derivatives a variant of some chemistry described by Meares et al (2) should be used. Standard peptide synthesis using the Rink resin along with FMOC protected natural and un-natural amino acids which can be conveniently cleaved at the penultimate step of the synthesis generates a tri-peptide C-terminal amide. This is reduced using Diborane in THF to give the open chain tetra-aza compounds as shown below:

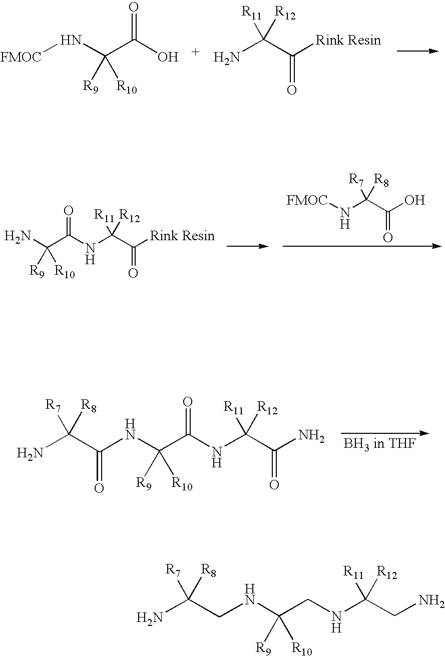

The incorporation of R₁, R2, R₅ and R₆ can be accomplished with this chemistry by standard procedures.

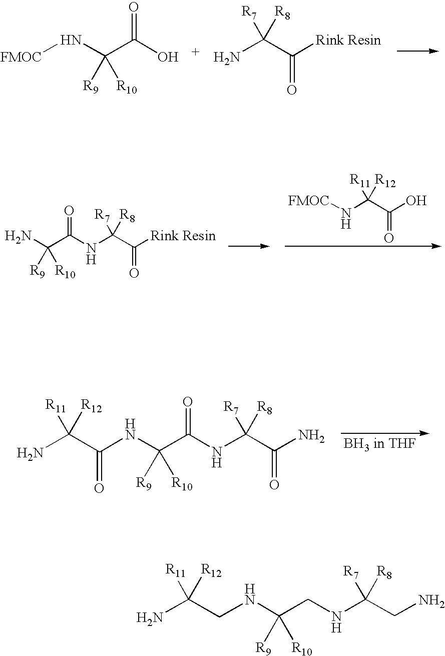

The reverse Rink approach, shown above, also leads to this class of tetra-aza derivatives and may be useful in cases where peptide coupling of a sterically hindered amino acid requires multiple coupling attempts in order to achieve success in the initial Rink approach.

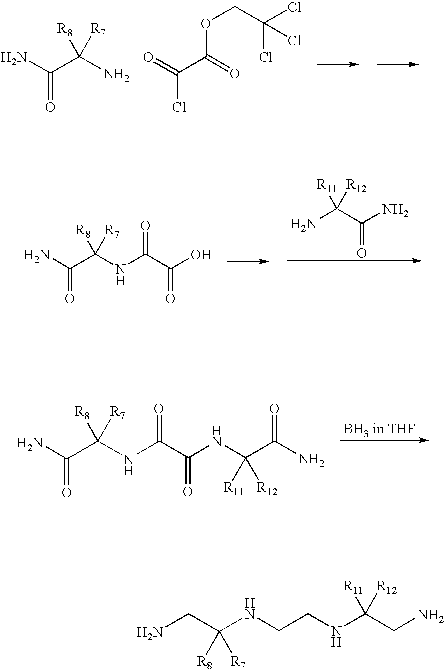

The oxalamide approach, shown above, also can lead to successful syntheses of this class of compounds, although the central substituents are always going to be hydrogen or its isotopes with this kind of chemistry. This particular variant makes use of the trichloroethyl ester group to protect one of the carbolxylic acid functions of oxalic acid but other protecting groups are also envisaged. Reaction of an aminoacid amide derived from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired tetra-aza derivative.

3NX Series 1:
when X1, X2, X3, are N and X4 is S or O then:
R6 does not exist
R1, R2, R3, R4 and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, and n3 are independently chosen to be 2 or 3, and each repeat of any of n1, n2, and n3 may be the same as or different than any other repeat; and
R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R1, R2, R3, R4, or R5 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NE-peptide, C1-C10 alkyl-NH-protein, C1-C10 alkyl-NH—CO-PEG, C1-C10 alkyl-S-peptide, C1-C10 alkyl-S-protein.

Furthermore one or several of R7, R8, R9, R10, R11, or R12 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Open Chain 3NX Series 1 of Formula I

Variations of the syntheses used for the 4N series provide examples of the 3N series 1 class of compounds. The chemistry described by Meares et al (2) can be modified to give examples of the 3NX series of compounds.

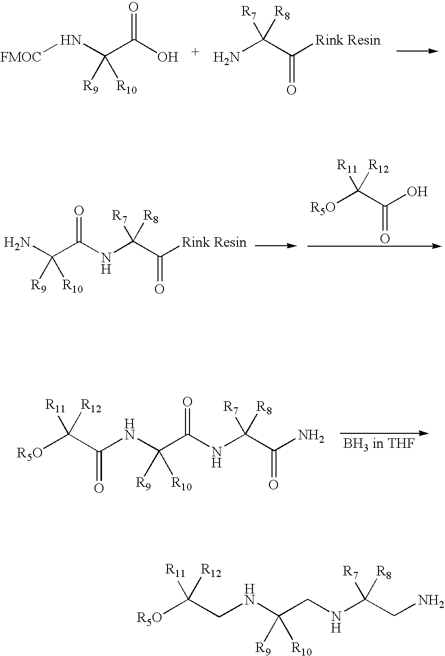

Standard peptide synthesis according to the so-called reverse Rink approach as shown above using FMOC protected natural and un-natural amino acids which can be conveniently cleaved at the penultimate step of the synthesis generates a modified tri-peptide C-terminal amide. The cases where X4 is O are incorporated by the use of an alpha-substituted carboxylic acid in the last coupling step. This is reduced using Diborane in THF to give the open chain tetra-aza compounds.

The incorporation of R₁, R2, R₅ and R₆ can be accomplished with this chemistry by standard procedures.

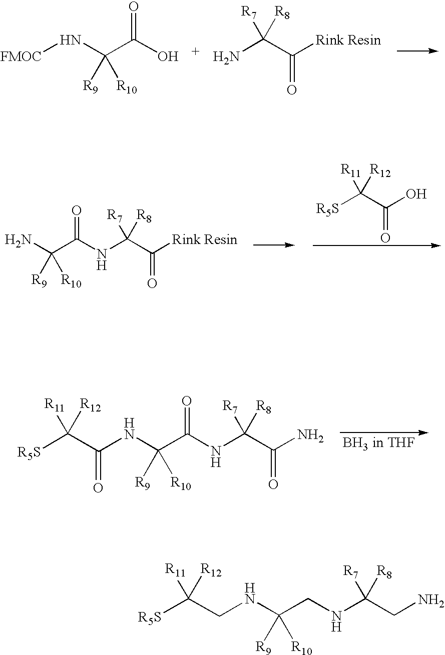

For the cases where X4=S a similar approach using standard peptide synthesis according to the so-called reverse Rink approach as shown above can be used. Coupling with FMOC protected natural and un-natural amino acids, which can be conveniently cleaved at the penultimate step of the synthesis, generates a modified tri-peptide C-terminal amide. The incorporation of X4=S is achieved by the use of an alpha-substituted carboxylic acid in the last coupling step. This is reduced using Diborane in THF to give the open chain tetra-aza compounds.

The incorporation of R₁, R2, R₅ and R₆ can be accomplished with this chemistry by standard procedures.

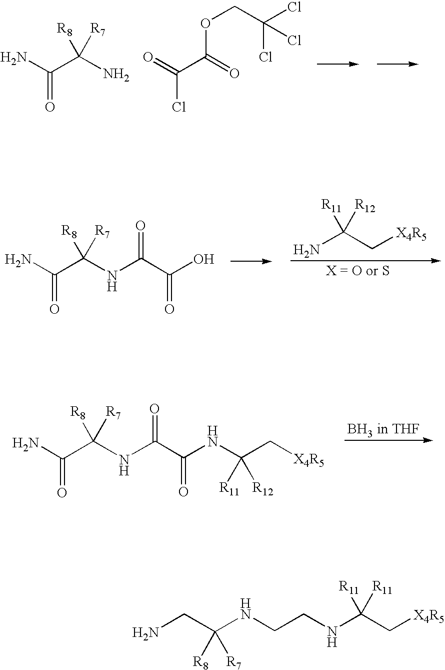

The oxalamide approach, shown above, can also lead to successful syntheses of this class of compounds, although the central substituents are always going to be hydrogen or its isotopes with this kind of chemistry. This particular variant makes use of the trichloroethyl ester group to protect one of the carbolxylic acid functions of oxalic acid but other protecting groups are also envisaged. Reaction of an aminoacid amide derived from a natural or unnatural amino acid with a differentially protected oxalyl mono chloride gives the mono-oxalamide shown which can be reacted under standard peptide coupling conditions with an ethanolamine or ethanethiolamine derivative to give the un-symmetrical bis-oxalamide which can then be reduced with diborane as shown to give the desired tri-aza derivative.

3NX Series 2:
when X1, X2, and X4 are N and X3 is O or S then:
R4 does not exist, and
R1, R2, R3, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, and n3 are independently chosen to be 2 or 3, and each repeat of any of n1, n2, and n3 may be the same as or different than any other repeat; and
R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R1, R2, R3, R5, or R6 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R7, R8, R9, R10, R11, or R12 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Open Chain 3NX Series 2 of Formula I

A different approach can be used for the synthesis of the 3N series 2 class of compounds. The key component is the incorporation in the synthesis of an appropriately substituted and protected ethanolamine or ethanethiolamine derivative, which is readily available from both natural and un-natural amino acids, as shown below.

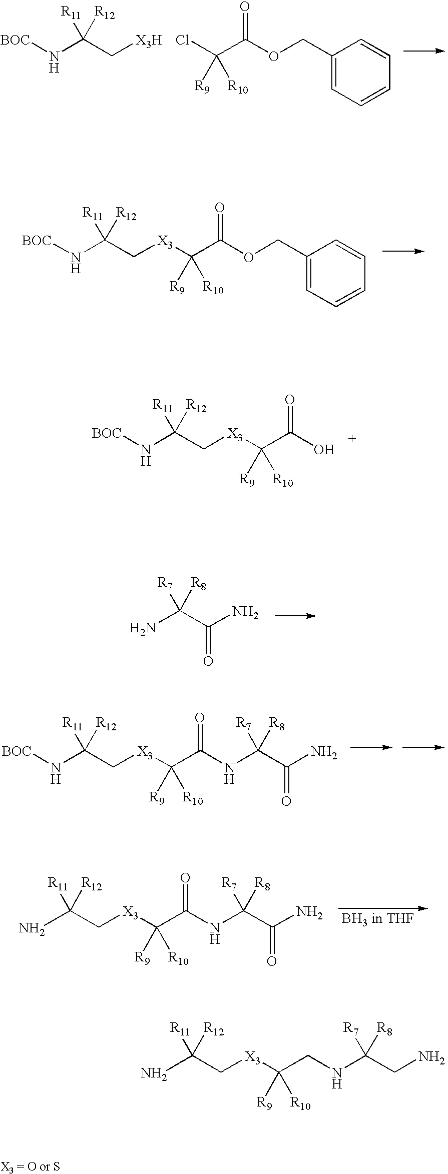

The BOC protected ethanolamine or ethanethiolamine is reacted with an appropriate benzyl protected alpha chloroacid. After hydrogenation to deprotect the ester function, standard peptide coupling with a natural or unnatural aminoacid amide followed by deprotection and reduction with diborane in THF gives the open chain tri-aza compounds. If hydrogenation is not compatible with other functionality in the molecule then alternative combinations of protecting groups can be used such as trichloroethyloxy carbonyl and t-butyl.

The incorporation of R₁, R2, R₅ and R₆ can be accomplished with this chemistry by standard procedures.

2N2X Series 1:
when X2 and X3 are N and X1 and X4 are O or S then:
R1 and R6 do not exist;
R2, R3, R4, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, and n3 are independently chosen to be 2 or 3, and each repeat of any of n1, n2, and n3 may be the same as or different than any other repeat; and
R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl

In addition, one or several of R2, R3, R4, or R5 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, R10, R11, or R12 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Open Chain 2N2X Series 1 of Formula I

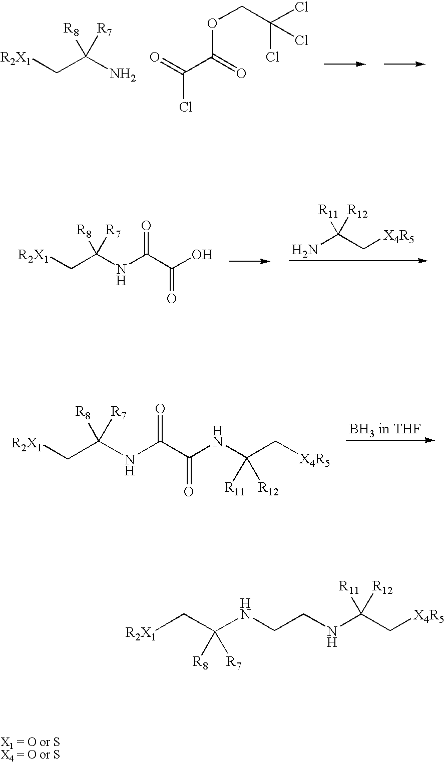

The oxalamide approach, shown above, can lead to successful syntheses of this class of compounds. This particular variant makes use of the trichloroethyl ester group to protect one of the carbolxylic acid functions of oxalic acid but other protecting groups are also envisaged. Reaction of an aminoalcohol or aminothiol derivative readily available from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired tetra-aza derivative.

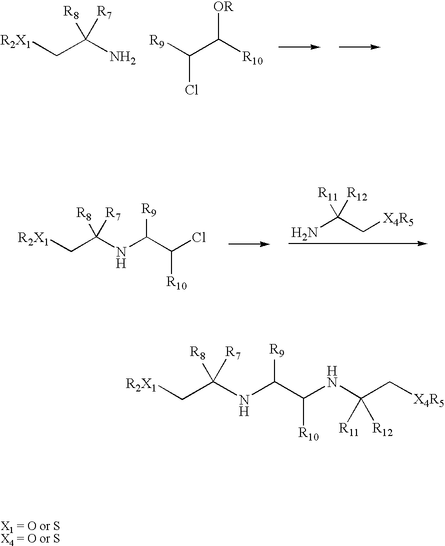

A variant of the dichloroethane approach, shown above, can also lead to successful syntheses of this class of compounds. Reaction of an aminoalcohol or aminothiol derivative readily available from a natural or unnatural amino acid with an O-protected 1-chloro, 2-hydroxy ethane derivative followed by deprotection and substitution with chloride gives the mono-chloro compound shown which can be further reacted with an appropriate aminoalcohol or aminothiol derivative readily available from a natural or unnatural amino acid to give the un-symmetrical desired product.

2N2X Series 2:
when X1 and X3 are N and X2 and X4 are O or S then:
R3 and R6 do not exist;
R1, R2, R4, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO₃H, CH2PO(OH)2, CH2P(CH3)O(OH);
n1, n2, and n3 are independently chosen to be 2 or 3, and each repeat of any of n1, n2, and n3 may be the same as or different than any other repeat; and
R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R1, R2, R4, or R5 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R7, R8, R9, R10, R11, or R12 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of the Open Chain 2N2X Series 2 of Formula I

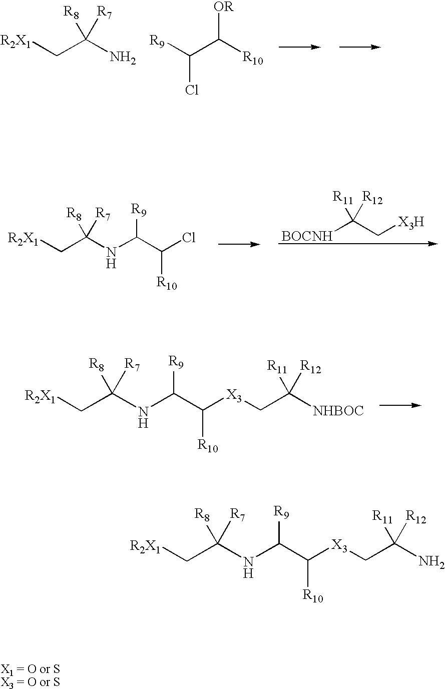

A variant of the dichloroethane approach, shown above, can lead to successful syntheses of this class of compounds. Reaction of an aminoalcohol or aminothiol derivative readily available from a natural or unnatural amino acid with an O-protected 1-chloro, 2-hydroxy ethane derivative followed by deprotection and substitution with chloride gives the mono-chloro compound shown which can be further reacted with an appropriately protected aminoalcohol or aminothiol derivative, readily available from a natural or unnatural amino acid, to give the un-symmetrical desired product after de-protection.

2N2X Series 3:
when X1 and X2 are N and X3 and X4 are O or S then:
R4 and R6 do not exist;
R1, R2, R3, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, and n3 are independently chosen to be 2 or 3, and each repeat of any of n1, n2, and n3 may be the same as or different than any other repeat; and
R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R1, R2, R3, or R5 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, R10, R11, or R12 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2N2X Series 3

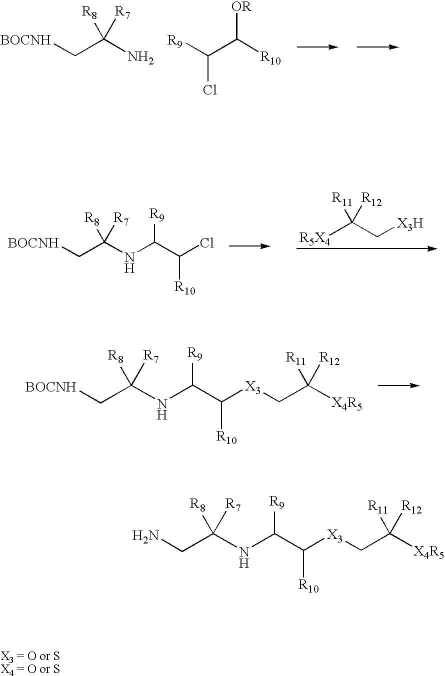

A variant of the dichloroethane approach, shown above, can lead to successful syntheses of this class of compounds. Reaction of a monoprotected ethylene diamine derivative, readily available from a natural or unnatural amino acid with an O-protected 1-chloro, 2-hydroxy ethane derivative followed by deprotection and substitution with chloride gives the mono-chloro compound shown which can be further reacted with an appropriately protected bis-alacohol or bis thiol derivative, readily available from a natural or unnatural amino acid, to give the un-symmetrical desired product after de-protection.

2N2X Series 4:
when X1 and X4 are N and X2 and X3 are O or S then:
R3 and R4 do not exist;
R1, R2, R5 and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, and n3 are independently chosen to be 2 or 3, and each repeat of any of n1, n2, and n3 may be the same as or different than any other repeat; and
R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R1, R2, R5, or R6 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, R10, R11, or R12 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2N2X Series 4 of Formula I

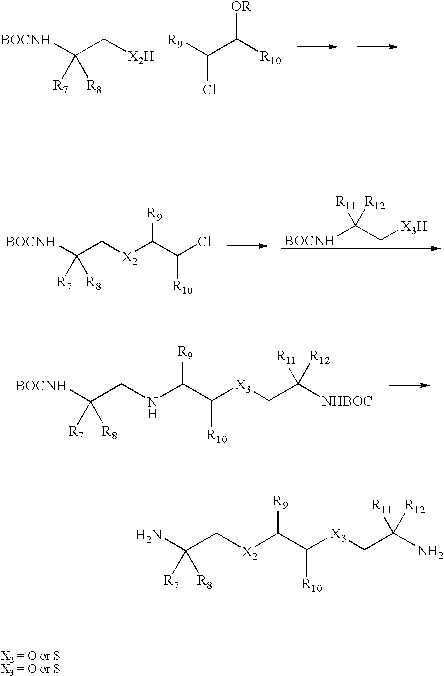

A variant of the dichloroethane approach, shown above, can lead to successful syntheses of this class of compounds. Reaction of a an appropriately protected bis-alacohol or bis thiol derivative, readily available from a natural or unnatural amino acid, with an O-protected 1-chloro, 2-hydroxy ethane derivative followed by deprotection and substitution with chloride gives the mono-chloro compound shown which can be further reacted with an appropriately protected bis-alacohol or bis thiol derivative, readily available from a natural or unnatural amino acid, to give the un-symmetrical desired product after de-protection.

For the Tetra-Heteroatom Cyclic Series:
R1 and R6 are joined together to form the bridging group (CR13R14)ₙ₄;
X1, X2, X3, and X4 are independently chosen from the atoms N, S or O such that:

4N Macrocyclic Series:
when X1, X2, X3, and X4 are N then:
R2, R3, R4, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, n3, and n4 are independently chosen to be 2 or 3, and each repeat of any of n1, n2, n3 and n4 may be the same as or different than any other repeat; and
R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R2, R3, R4, or R5 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, R10, R11, R12, R13 or R14 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 4N Series of Formula I

Trientine itself has been synthesized by reaction of 2 equivalents of ethylene diamine with 1,2-dichloro ethane to give trientine directly (1). Possible side products from this synthesis include the 12N4 macrocycle shown below, which could also be synthesized directly from Trientine by reaction with a further equivalent of 1,2-dichloro ethane under appropriately dilute concentrations to provide the 12N4 macrocycle shown. Modification of this procedure by using starting materials with appropriate R groups would lead to symmetrically substituted 12N4 macrocycle examples as shown below:

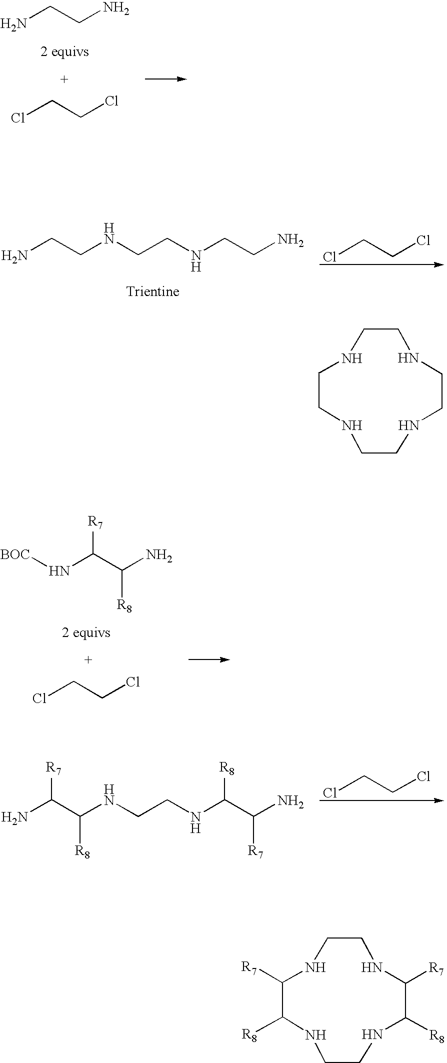

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the tetra-aza series. In order to obtain the un-symmetrically substituted derivatives a variant of some chemistry described by Meares et al (2) should be used. Standard peptide synthesis using the Merrifield approach or the SASRIN resin along with FMOC protected natural and un-natural amino acids which can be conveniently cleaved at a later step of the synthesis generates a fully protected tetra-peptide C-terminal SASRIN derivative. Cleavage of the N terminal FMOC protecting group followed by direct cyclization upon concomitant cleavage from the resin gives the macrocyclic tetrapeptide. This is reduced using Diborane in THF to give the 12N4 series of compounds as shown below:

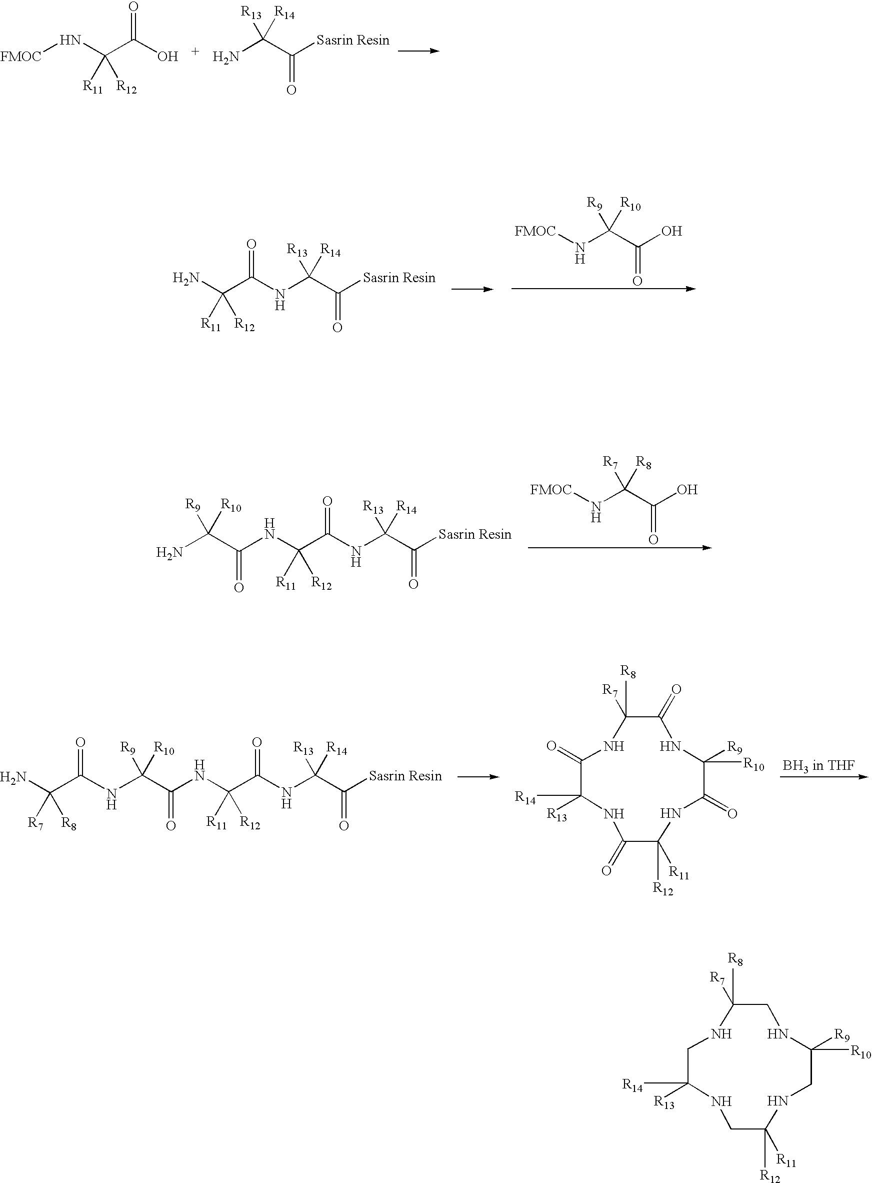

The incorporation of R₁, R2, R₅ and R₆ can be accomplished with this chemistry by standard procedures.

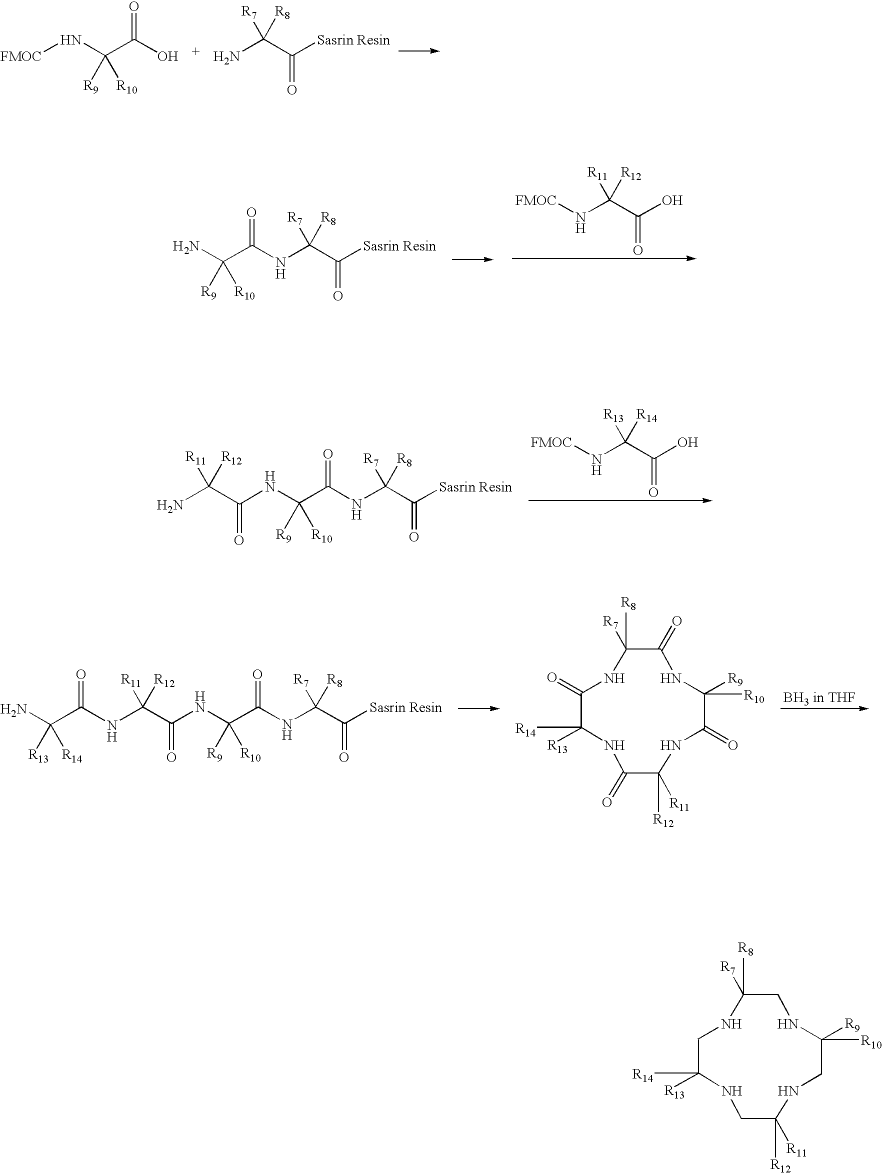

The reverse Merrifield/SASRIN approach, shown above, also leads to this class of tetra-aza derivatives and may be useful in cases where peptide coupling of a sterically hindered amino acid requires multiple coupling attempts in order to achieve success in the initial Merrifield approach.

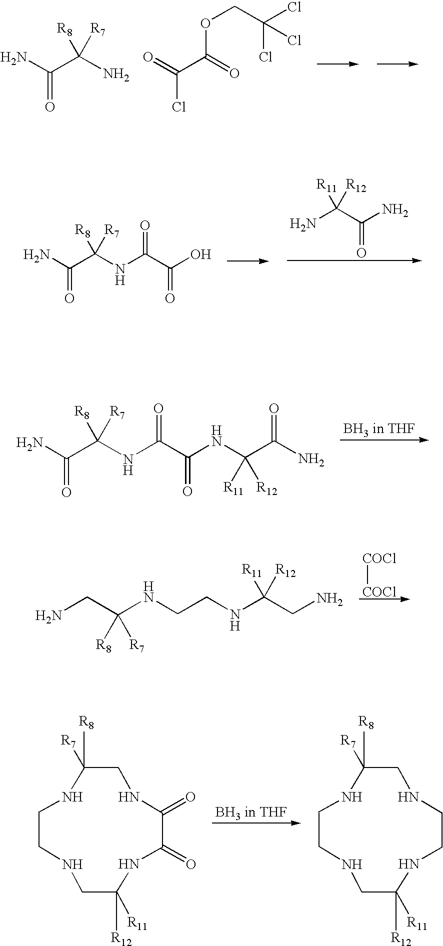

The oxalamide approach, shown above, also can lead to successful syntheses of this class of compounds. This particular variant makes use of the trichloroethyl ester group to protect one of the carbolxylic acid functions of oxalic acid but other protecting groups are also envisaged. Reaction of an aminoacid amide derived from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired tetra-aza derivative. Further reaction with oxalic acid gives the cyclic derivative, which can then be reduced once again with diborane to give the 12N4 series of compounds.

3NX Series:
when X1, X2, X3, are N and X4 is S or O then:
R5 does not exist;
R2, R3, and R4 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, n3, and n4 are independently chosen to be 2 or 3, and each repeat of any of n1, n2, n3 and n4 may be the same as or different than any other repeat; and
R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R2, R3 or R4 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, R10, R11, R12, R13 or R14 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 3NX Series of Formula I

Trientine itself has been synthesized by reaction of 2 equivalents of ethylene diamine with 1,2-dichloro ethane to give trientine directly (1). Possible side products from this synthesis include the 12N4 macrocycle shown below, which could also be synthesized directly from Trientine by reaction with a further equivalent of 1,2-dichloro ethane under appropriately dilute concentrations to provide the 12N4 macrocycle shown. Modification of this procedure by using starting materials with appropriate R groups leads to symmetrically substituted 12N4 macrocycle examples as shown below:

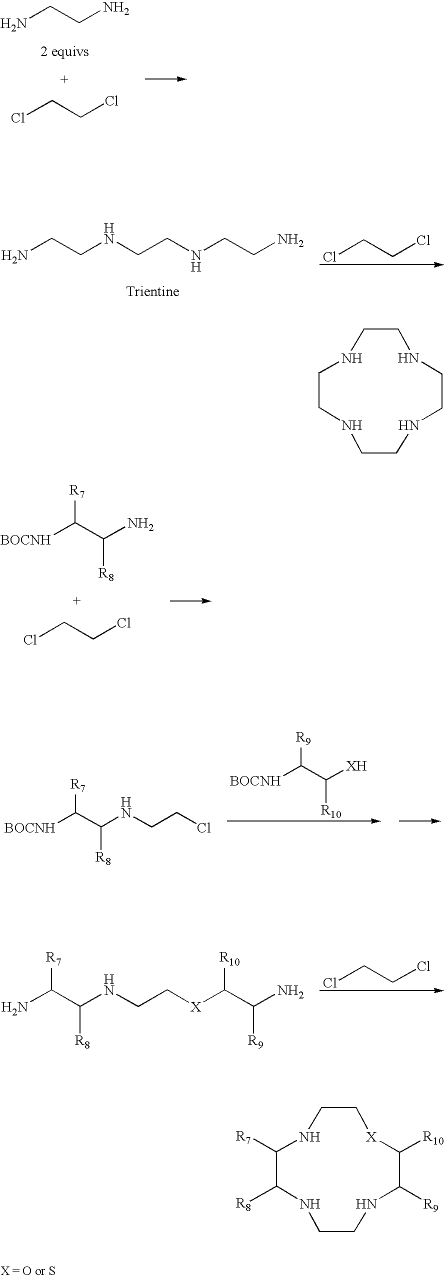

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the tri-aza X series. In order to obtain alternative un-symmetrically substituted derivatives a variant of some chemistry described by Meares et al (2) could be used. Standard peptide synthesis using the Merrifield approach or the SASRIN resin along with FMOC protected natural and un-natural amino acids which can be conveniently cleaved at a later step of the synthesis generates a tri-peptide C-terminal SASRIN derivative which can be further elaborated with an appropriate BOCO or BOCS compound the give the resin bouond 3NX compound shown. Reduction with diborane followed by Tosylation would give the 3NX OTosyl linear compound, which, upon deprotection and cyclization would give the desired 3NX macrocycle as shown below:

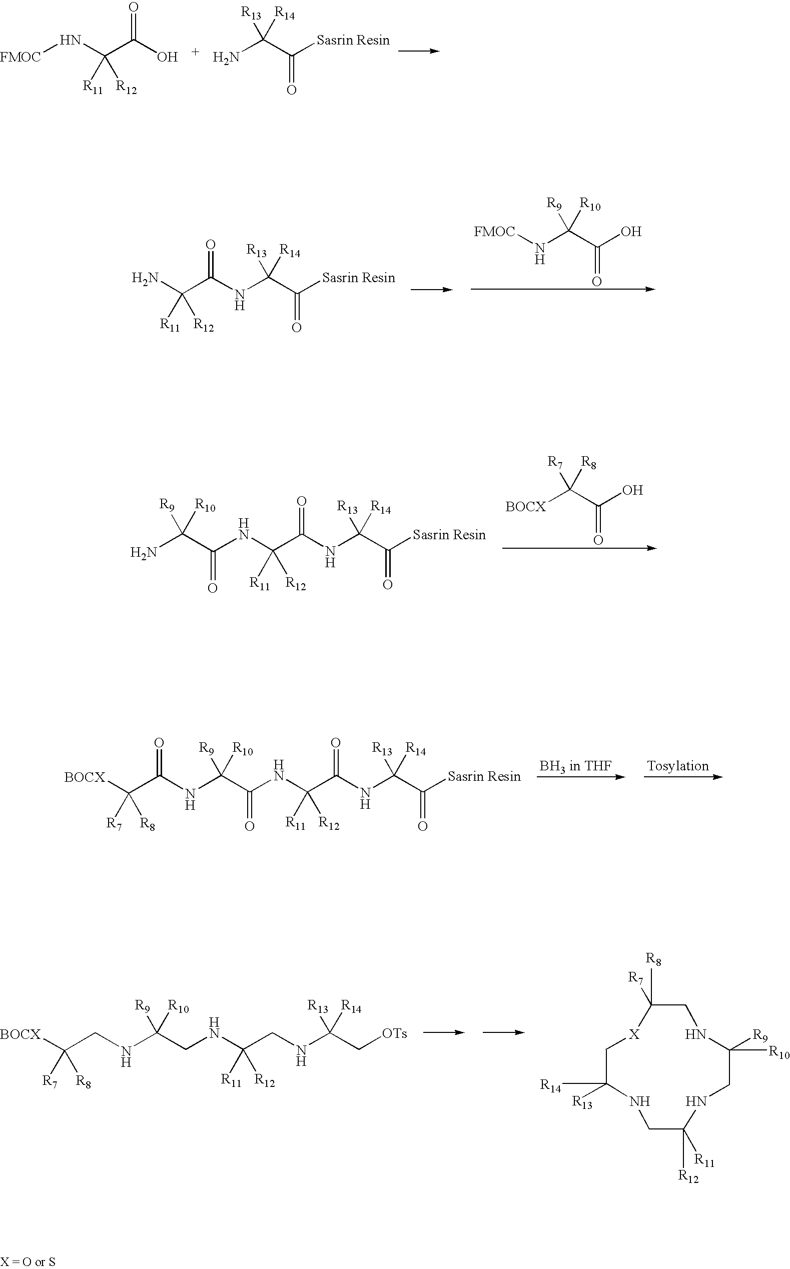

The incorporation of R₁, R2, R₅ and R₆ can be accomplished with this chemistry by standard procedures.

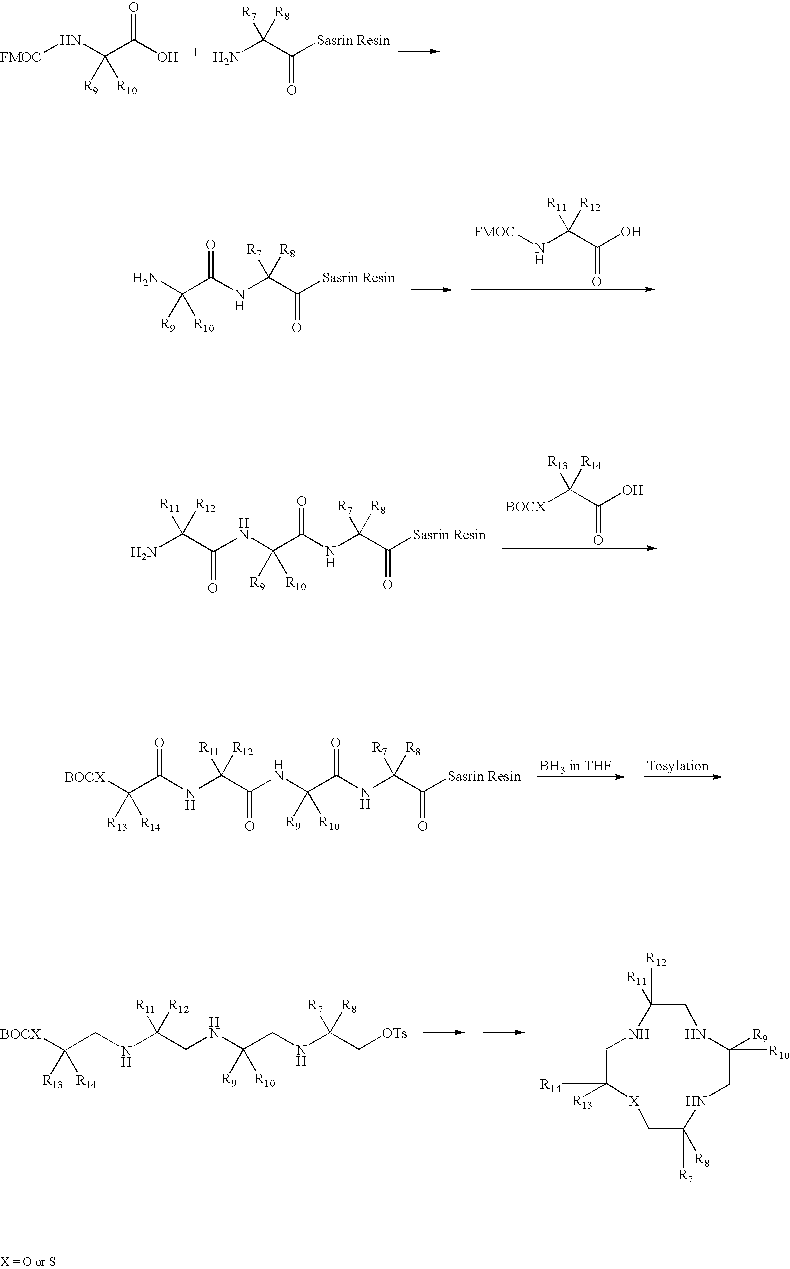

The reverse Merrifield/SASRIN approach, shown above, also leads to this class of tetra-aza derivatives and may be useful in cases where peptide coupling of a sterically hindered amino acid requires multiple coupling attempts in order to achieve success in the initial Merrifield approach.

2N2X Series 1:
when X2 and X3 are N and X1 and X4 are O or S then:
R2 and R5 do not exist
R3 and R4 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, n3, and n4 are independently chosen to be 2 or 3, and each repeat of any of n1, n2, n3 and n4 may be the same as or different than any other repeat; and
R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl

In addition, one or both of R3, or R4 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R7, R8, R9, R10, R11, R12, R13 or R14 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 2N2X Series 1 of Formula I

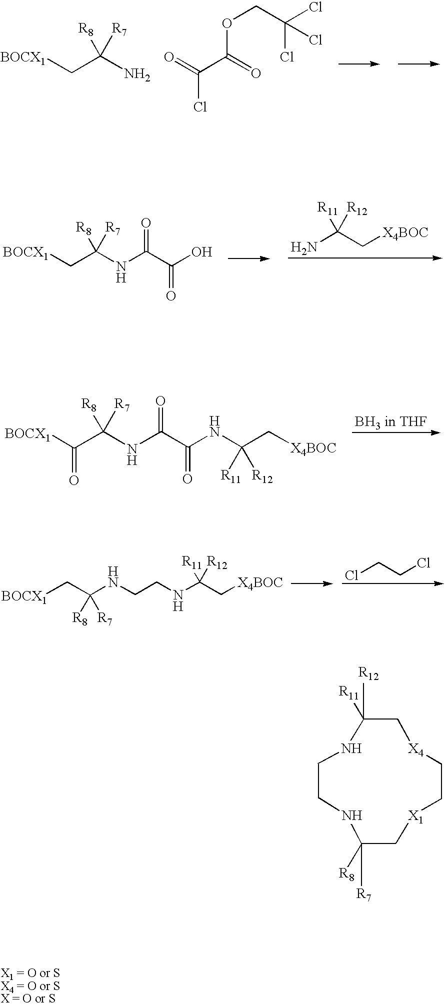

The oxalamide approach, shown above, again can lead to successful syntheses of this class of compounds, although the central substituents are always going to be hydrogen or its isotopes with this kind of chemistry. This particular variant makes use of the trichloroethyl ester group to protect one of the carboxylic acid functions of oxalic acid but other protecting groups are also envisaged. Reaction of an aminoalcohol or aminothiol derivative readily available from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired di-aza derivative. Deprotection followed by cyclization would give the 12N2X2 analogs.

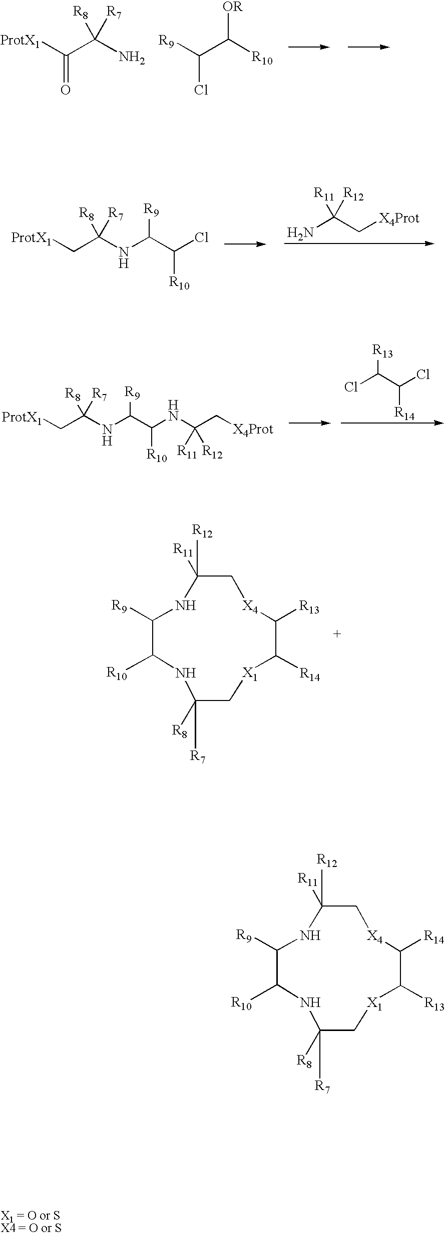

A variant of the dichloroethane approach, shown above, can also lead to successful syntheses of this class of compounds. Reaction of an aminoalcohol or aminothiol derivative readily available from a natural or unnatural amino acid with an O-protected 1-chloro, 2-hydroxy ethane derivative followed by deprotection and substitution with chloride gives the mono-chloro compound shown which can be further reacted with an appropriate aminoalcohol or aminothiol derivative readily available from a natural or unnatural amino acid to give the un-symmetrical shown. Deprotection followed by cyclization with a dichloroethan derivative would give a mixture of the the two position isomers shown.

2N2X Series 2:
when X1 and X3 are N and X2 and X4 are O or S then:
R3 and R5 do not exist
R2 and R4 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, n3, and n4 are independently chosen to be 2 or 3, and each repeat of any of n1, n2, n3 and n4 may be the same as or different than any other repeat; and
R7, R8, R9, R10, R1, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or both of R2, or R4 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R7, R8, R9, R10, R11, R12, R13 or R14 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 2N2X Series 2 of Formula I

Trientine itself has been synthesized by reaction of 2 equivalents of ethylene diamine with 1,2-dichloro ethane to give trientine directly (1). Possible side products from this synthesis include the 12N4 macrocycle shown below, which could also be synthesized directly from Trientine by reaction with a further equivalent of 1,2-dichloro ethane under appropriately dilute concentrations to provide the 12N4 macrocycle shown. Modification of this procedure by using starting materials with appropriate R groups would lead to symmetrically substituted 12N4 macrocycle examples as shown below:

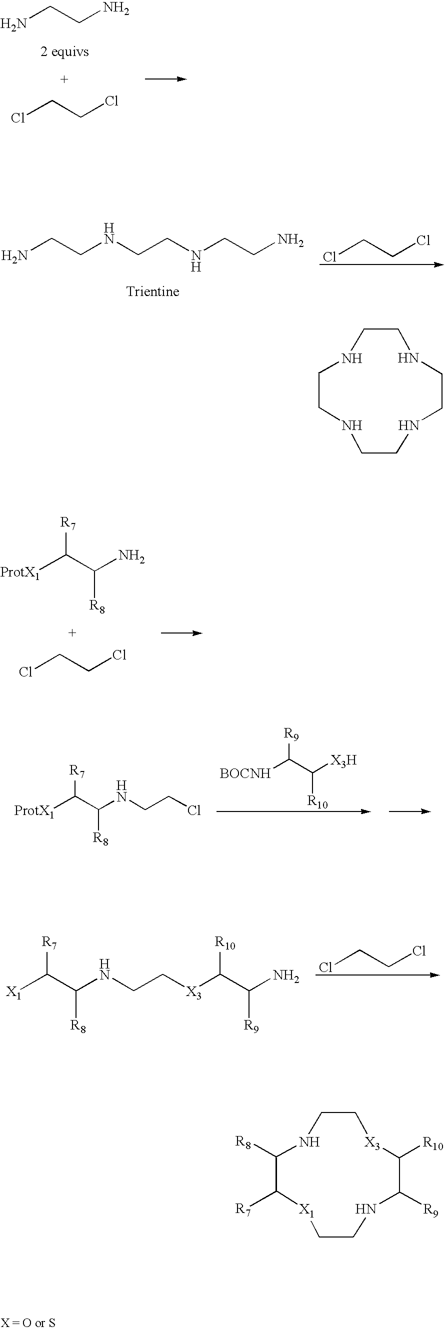

The judicious use of protecting group chemistry such as the widely used BOC (t-butyloxycarbonyl) group and an appropriate O or S protecting group allows the chemistry to be directed specifically towards the substitution pattern shown. Other approaches such as via the chemistry of ethyleneimine (2) may also lead to a subset of the di-aza 2X series. A variant of this approach using substituted dichloroethane derivatives could be used to access more complex substitution patterns. This would lead to mixtures of position isomers, which can be separated by HPLC.

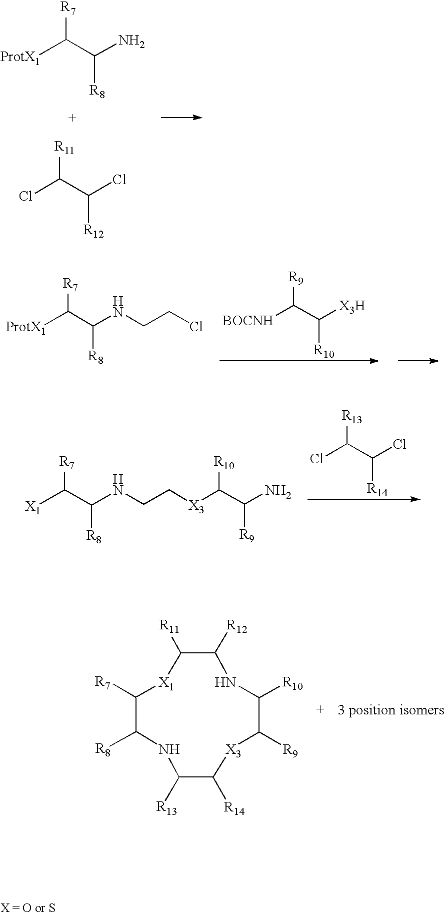

1N3X Series:
when X1 is N and X2, X3 and X4 are O or S then:
R3, R4 and R5 do not exist;
R2 is independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, n3, and n4 are independently chosen to be 2 or 3, and each repeat of any of n1, n2, n3 and n4 may be the same as or different than any other repeat; and
R7, R8, R9, R10, R11, R12, R13 and R14 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, R2 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, R10, R11, R12, R13 or R14 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 1N3X Series of Formula I

Trientine itself has been synthesized by reaction of 2 equivalents of ethylene diamine with 1,2-dichloro ethane to give trientine directly (1). Possible side products from this synthesis include the 12N4 macrocycle shown below, which could also be synthesized directly from Trientine by reaction with a further equivalent of 1,2-dichloro ethane under appropriately dilute concentrations to provide the 12N4 macrocycle shown. Modification of this procedure by using starting materials with appropriate R groups would lead to substituted 12NX3 macrocycle examples as shown below:

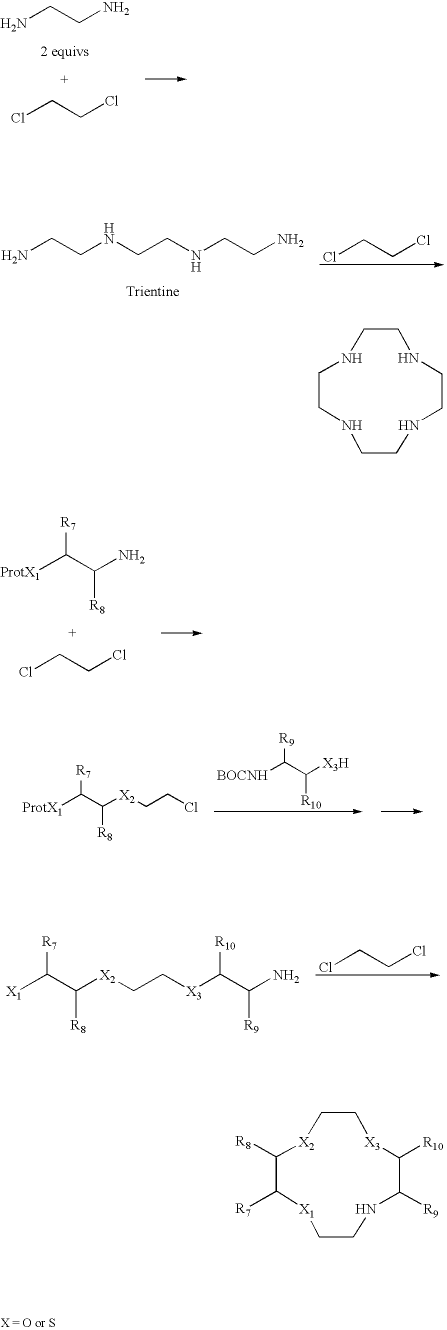

The judicious use of protecting group chemistry such as the widely used BOC (t-butyloxycarbonyl) group and an appropriate O or S protecting group allows the chemistry to be directed specifically towards the substitution pattern shown. Other approaches such as via the chemistry of ethyleneimine (2) may also lead to a subset of the mono-aza 3X series. A variant of this approach using substituted dichloroethane derivatives could be used to access more complex substitution patterns. This would lead to mixtures of position isomers, which can be separated by HPLC.

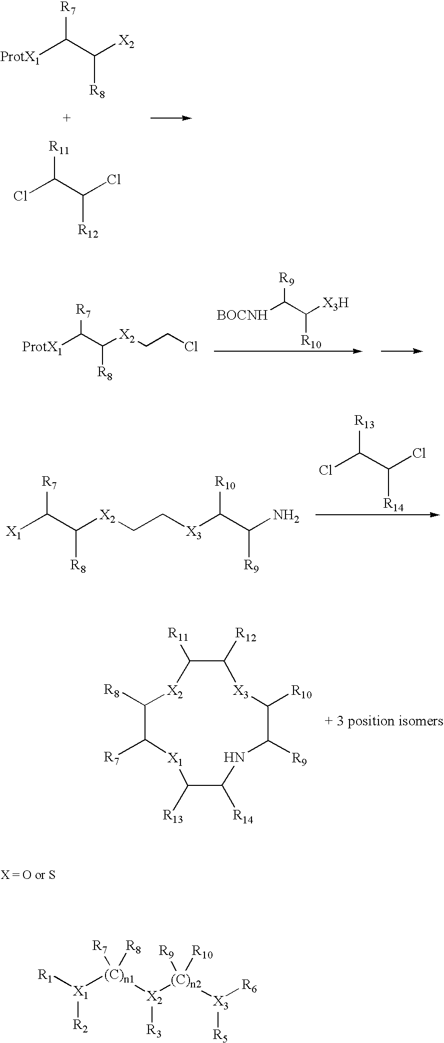

For the Tri-Heteroatom Acyclic Examples of Formula II:
X1, X2, and X3 are independently chosen from the atoms N, S or O such that:

3N Series:
when X1, X2, and X3 are N then:
R1, R2, R3, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1 and n2 are independently chosen to be 2 or 3, and each repeat of any of n1 and n2 may be the same as or different than any other repeat; and
R7, R8, R9, and R10 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R1, R2, R3, R5 or R6 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R7, R8, R9, or R10 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 3N Series of Formula II

As mentioned above Trientine itself has been synthesized by reaction of 2 equivalents of ethylene diamine with 1,2-dichloro ethane to give Trientine directly (1). A variant of this procedure by using starting materials with appropriate R groups and 1-amino,2-chloro ethane would lead to some open chain 3N examples as shown below:

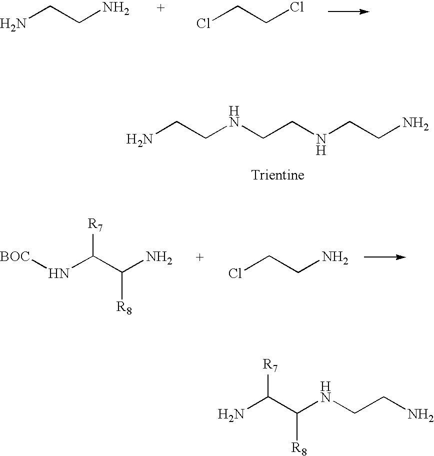

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the tri-aza series. In order to obtain the un-symmetrically substituted derivatives a variant of some chemistry described by Meares et al (2) could be used. Standard peptide synthesis using the Rink resin along with FMOC protected natural and un-natural amino acids which can be conveniently cleaved at the penultimate step of the synthesis generates a di-peptide C-terminal amide. This can be reduced using Diborane in THF to give the open chain tri-aza compounds as shown below:

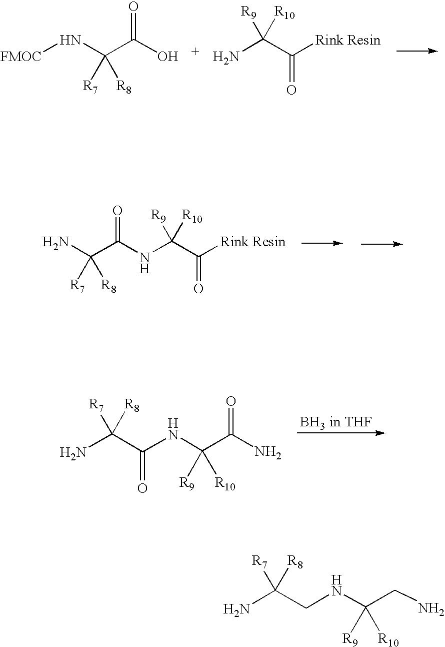

The reverse Rink approach may also be useful where peptide coupling is slowed for a particular substitution pattern as shown below. Again the incorporation of R₁, R2, R₅ and R₆ can be accomplished with this chemistry by standard procedures:

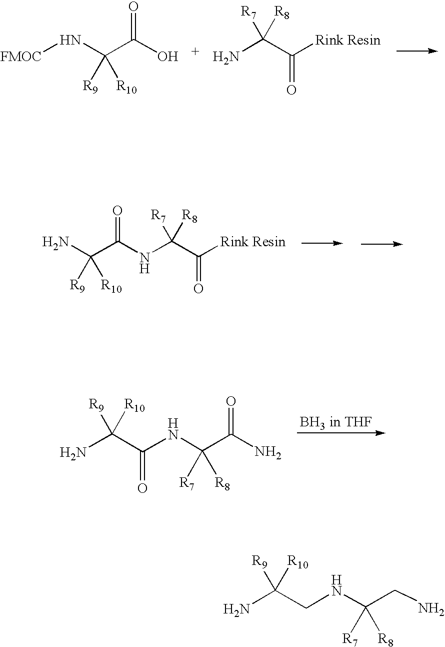

2NX Series 1:
when X1 and X3 are N and X2 is S or O then:
R3 does not exist
R1, R2, R5, and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1 and n2 are independently chosen to be 2 or 3, and each repeat of any of n1 and n2 may be the same as or different than any other repeat; and
R7, R8, R9, and R10 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl

In addition, one or several of R1, R2, R5 or R6 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, or R10 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2NX Series 1 of Formula II

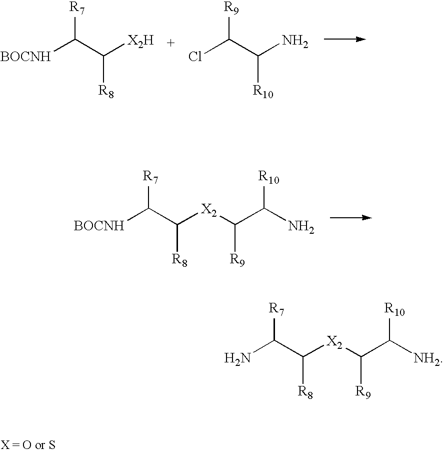

The synthesis of the 2NX series 1 compounds can be readily achieved as shown above. The judicious use of protecting group chemistry such as the widely used BOC (t-butyloxycarbonyl) group allows the chemistry to be directed specifically towards the substitution pattern shown above. Other approaches such as via the chemistry of ethyleneimine (2) may also lead to a subset of the tri-aza X series.

2NX Series 2
when X1 and X2 are N and X3 is O or S then:
R5 does not exist;
R1, R2, R3 and R6 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1 and n2 are independently chosen to be 2 or 3, and each repeat of any of n1 and n2 may be the same as or different than any other repeat; and
R7, R8, R9, and R10 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R1, R2, R5, or R6 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, or R10 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2NX Series 2 of Formula II

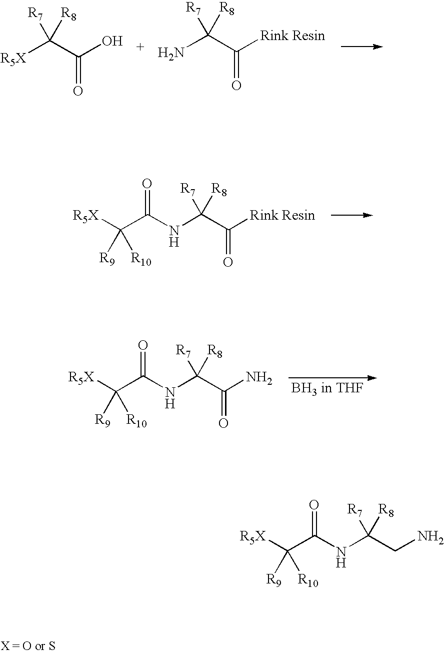

For the cases where X═O or S a similar approach using standard peptide synthesis according to the Rink approach as shown above can be used. Coupling of a suitably protected alpha thiolo or hydroxy carboxylic acid with a Rink resin amino acid derivative followed by cleavage gives the desired linear di-amide, which can be reduced with Diborane in THF to give the open chain 2NX compounds.

The incorporation of R₁, R2, R₅ and R₆ can be accomplished with this chemistry by standard procedures.

The reverse Rink version is also feasible and again the incorporation of R₁, R2, R₅ and R₆ can be accomplished with this chemistry by standard procedures.

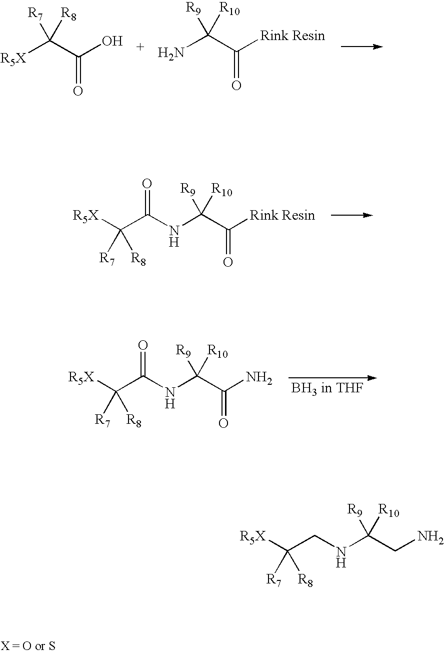

Tri-Heteroatom Cyclic Series of Formula II:
R1 and R6 form a bridging group (CR11R12)n3; and
X1, X2, and X3 are independently chosen from the atoms N, S or O such that:

3N Series:
when X1, X2 and X3 are N then:
R2, R3, and R5 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, and n3 are independently chosen to be 2 or 3, and each repeat of any of n1, n2 and n3 may be the same as or different than any other repeat; and
R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R2, R3, or R5 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, R10, R11, or R12 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 3N Series of Formula II

As mentioned above Trientine itself has been synthesized by reaction of 2 equivalents of ethylene diamine with 1,2-dichloro ethane to give Trientine directly (1). A variant of this procedure by using starting materials with appropriate R groups and 1-amino,2-chloro ethane would lead to open chain 3N examples which could then be cyclized by reaction with an appropriate 1,2 dichloroethane derivative as shown below:

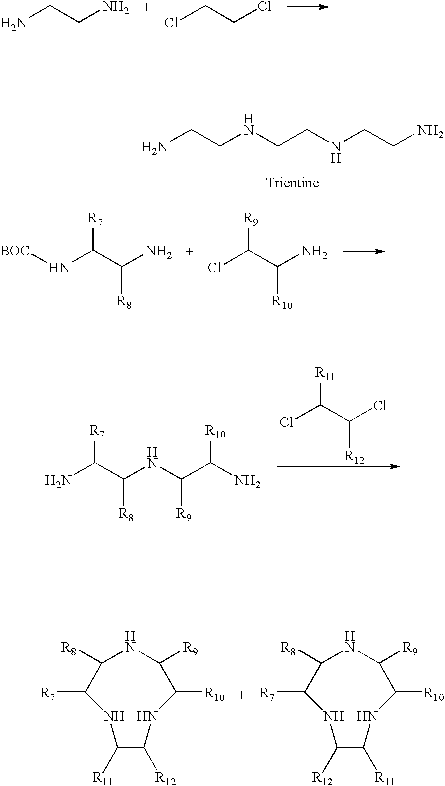

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the macrocyclic tri-aza series. In order to obtain the un-symmetrically substituted derivatives a variant of some chemistry described by Meares et al (2) could be used. Standard peptide synthesis using the Merrifield approach/SASRIN resin along with FMOC protected natural and un-natural amino acids which can be conveniently cleaved at the penultimate step of the synthesis generates a tri-peptide attached to resin via it's C-terminus. This can be cyclized during concomitant cleavage from the resin followed by reduction using Diborane in THF to give the cyclic tri-aza compounds as shown below:

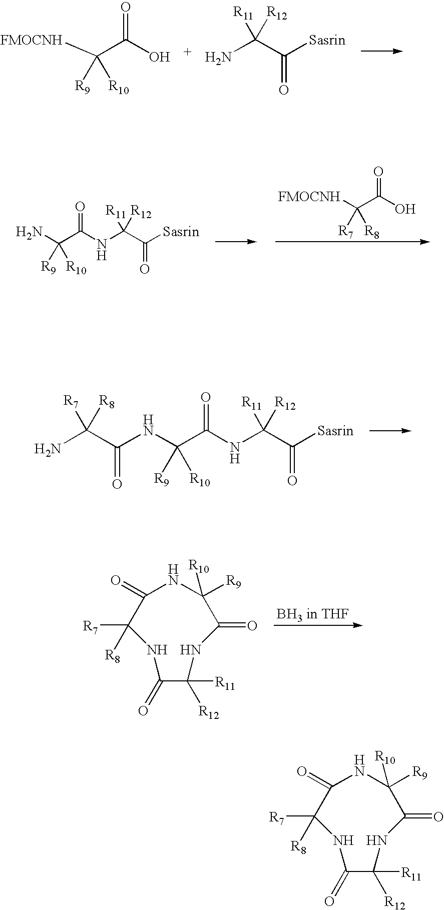

The incorporation of R₁, R₂, and R₅ can be accomplished with this chemistry by standard procedures.

The reverse Rink approach may also be useful where peptide coupling is slowed for a particular substitution pattern as shown below. Again the incorporation of R₁, R2, R₅ and R₆ can be accomplished with this chemistry by standard procedures:

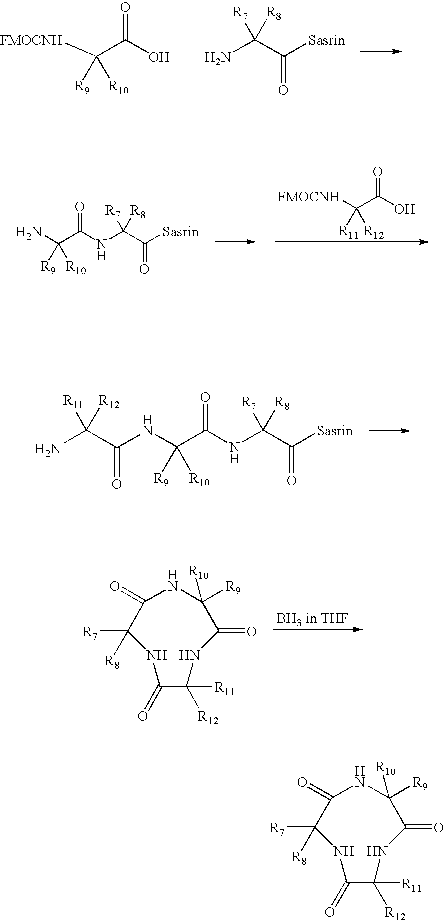

2NX Series:
when X1 and X2 are N and X3 is S or O then:
R5 does not exist;
R2 and R3 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, and n3 are independently chosen to be 2 or 3, and each repeat of any of n1, n2 and n3 may be the same as or different than any other repeat; and
R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl

In addition, one or both of R2 or R3 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, R10, R11, or R12 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 2NX Series of Formula II

As mentioned above Trientine itself has been synthesized by reaction of 2 equivalents of ethylene diamine with 1,2-dichloro ethane to give Trientine directly (1). A variant of this procedure by using starting materials with appropriate R groups and 1-amino,2-chloro ethane would lead to open chain 2NX examples which could then be cyclized by reaction with an appropriate 1,2 dichloroethane derivative as shown below:

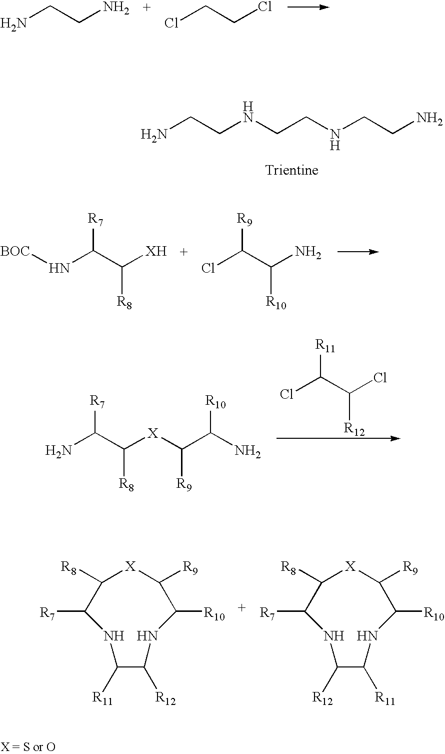

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the macrocyclic di-aza X series. In order to obtain the un-symmetrically substituted derivatives a variant of some chemistry described by Meares et al (2) could be used. Standard peptide synthesis using the Merrifield approach/SASRIN resin along with FMOC protected natural and un-natural amino acids which can be conveniently cleaved at the penultimate step of the synthesis generates a tri-peptide attached to resin via it's C-terminus. This can be cyclized during concomitant cleavage from the resin followed by reduction using Diborane in THF to give the cyclic tri-aza compounds as shown below:

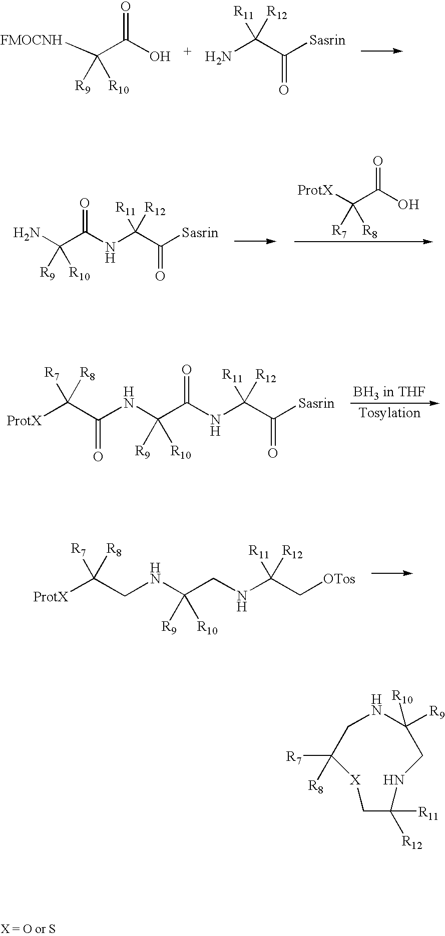

The incorporation of R₁, and R₂ can be accomplished with this chemistry by standard procedures.

The reverse Rink approach may also be useful where peptide coupling is slowed for a particular substitution pattern as shown below. Again the incorporation of R1, and R₂ can be accomplished with this chemistry by standard procedures:

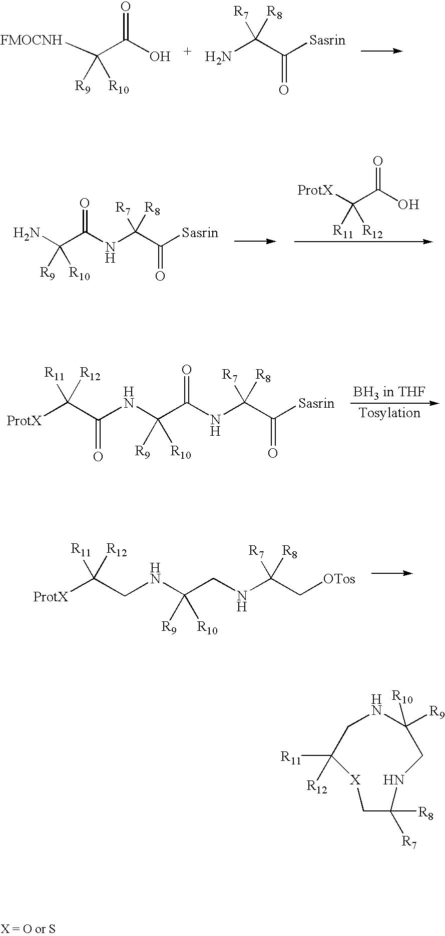

1N2X Series:
when X1 is N and X2 and X3 are O or S then:
R3 and R5 do not exist;
R2 is independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH2COOH, CH2SO3H, CH2PO(OH)2, CH2P(CH3)O(OH);
n1, n2, and n3 are independently chosen to be 2 or 3, and each repeat of any of n1, n2 and n3 may be the same as or different than any other repeat;
R7, R8, R9, R10, R11, and R12 are independently chosen from H, CH3, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, R2 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R7, R8, R9, R10, R11, or R12 may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 1N2X Series of Formula II

As mentioned above Trientine itself has been synthesized by reaction of 2 equivalents of ethylene diamine with 1,2-dichloro ethane to give Trientine directly (1). A variant of this procedure by using starting materials with appropriate R groups and 1-amino,2-chloro ethane would lead to open chain 1N2X examples which could then be cyclized by reaction with an appropriate 1,2 dichloroethane derivative as shown below:

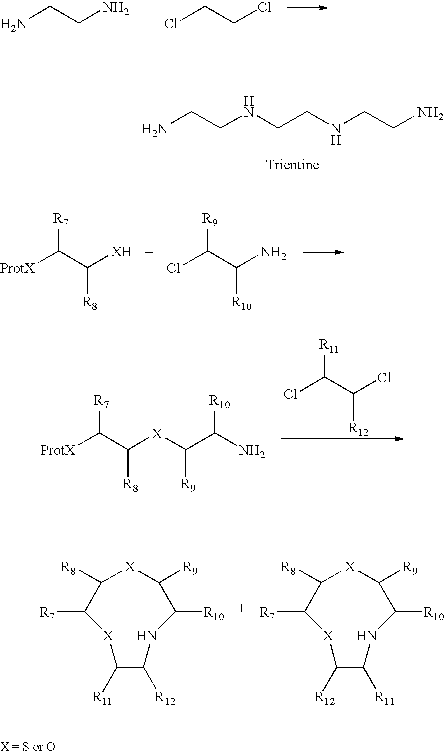

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the macrocyclic aza di-X series. In order to obtain the un-symmetrically substituted derivatives a variant of some chemistry above could be used:

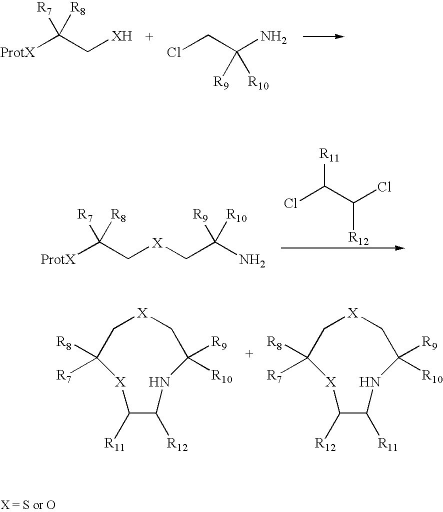

The incorporation of R₁ and R₂ can by accomplished with this chemistry by standard procedures.

Many of the synthetic routes allow for control of the particular R groups introduced. For synthetic methods incorporating amino acids, synthetic amino acids can be used to incorporate a variety of substituent R groups. The dichloroethane synthetic schemes also allow for the incorporation of a wide variety of R groups by using dichlorinated ethane derivatives. It will be appreciated that many of these synthetic schemes can lead to isomeric forms of the compounds; such isomers can be separated using techniques known in the art.

Documents describing aspects of these synthetic schemes include the following: (1) A W von Hoffman, Berichte 23, 3711 (1890); (2) The Polymerization Of Ethylenimine, Giffin D. Jones, Ame Langsjoen, Sister Mary Marguerite Christine Neumann, Jack Zomlefer, J. Org. Chem., 1944; 9(2); 125-147; (3) The peptide way to macrocyclic bifunctional chelating agents:synthesis of 2-(p-nitrobenzyl)-1,4,7,10-tetraazacyclododecane-N,N′,N″,N′″-tetraacetic acid and study of its yttrium(III) complex, Min K. Moi, Claude F. Meares, Sally J. DeNardo, J. Am. Chem. Soc.,1988; 110(18); 6266-6267; (4) Synthesis of a kinetically stable ⁹⁰Y labelled macrocycle-antibody conjugate, Jonathan P L Cox, Karl J Jankowski, Ritu Kataky, David Parker, Nigel R A Beeley, Byron A Boyce, Michael A W Eaton, Kenneth Millar, Andrew T Millican, Alice Harrison and Carole Walker, J. Chem. Soc. Chem. Comm., 797 (1989); (5) Specific and stable labeling of antibodies with technetium-99m with a diamide dithiolate chelating agent, Fritzberg A R, Abrams P G, Beaumier P L, Kasina S, Morgan A C, Rao T N, Reno J M, Sanderson J A, Srinivasan A, Wilbur D S, et al., Proc Natl Acad Sci USA. 1988 June; 85(11):4025-4029; (6) Towards tumour imaging with ¹¹¹In labelled macrocycle-antibody conjugates, Andrew S Craig, Ian M Helps, Karl J Jankowski, David Parker, Nigel R A Beeley, Byron A Boyce, Michael A W Eaton, Andrew T Millican, Kenneth Millar, Alison Phipps, Stephen K Rhind, Alice Harrison and Carol Walker, J. Chem. Soc. Chem. Comm., 794 (1989); (7) Synthesis of C- and N-functionalised derivatives of NOTA, DOTA, and DTPA:bifunctional complexing agents for the derivitisation of antibodies, Jonathan P L Cox, Andrew S Craig, Ian M Helps, Karl J Jankowski, David Parker, Michael A W Eaton, Andrew T Millican, Kenneth Millar, Nigel R A Beeley and Byron A Boyce, J. Chem. Soc. Perkin. I, 2567 (1990); (8) Macrocyclic chelators as anticancer agents in radioimmunotherapy, N R A Beeley and P R J Ansell, Current Opinions in Therapeutic Patents, 2 1539-1553 (1992); and (9) Synthesis of new macrocyclic amino-phosphinic acid complexing agents and their C— and P— functionalised derivatives for protein linkage, Christopher J Broan, Eleanor Cole, Karl J Jankowski, David Parker, Kanthi Pulukoddy, Byron A Boyce, Nigel R A Beeley, Kenneth Millar and Andrew T Millican, Synthesis, 63 (1992).

Any of the methods of treating a subject having or suspected of having or predisposed to a neurodegenerative disease, disorder, and/or condition, or other diseases, disorders, and/or conditions referenced or described herein may utilize the administration of any of the doses, dosage forms, formulations, compositions and/or devices herein described.

Aspects of the invention include controlled or other doses, dosage forms, formulations, compositions and/or devices containing one or more copper antagonists, for example, one or more compounds of Formulae I or II, or trientine active agents, including but not limited to, trientine, trientine dihydrochloride or other pharmaceutically acceptable salts thereof, trientine analogues of formulae I and II and salts thereof. The present invention includes, for example, doses and dosage forms for at least oral administration, transdermal delivery, topical application, suppository delivery, transmucosal delivery, injection (including subcutaneous administration, subdermal administration, intramuscular administration, depot administration, and intravenous administration (including delivery via bolus, slow intravenous injection, and intravenous drip), infusion devices (including implantable infusion devices, both active and passive), administration by inhalation or insufflation, buccal administration, sublingual administration, and ophthalmic administration.

Neurodegenerative disease, disorders and/or conditions in which the methods, uses, doses, dose formulations, and routes of administration thereof of the invention will be useful include, for example, dementia, memory impairment caused by dementia, memory impairment seen in senile dementia, various degenerative diseases of the nerves including Alzheimer's disease, Huntingtons disease, Parkinson's disease, parkinsonism, amyotrophic lateral sclerosis (ALS), Friedreich's ataxia and other hereditary ataxia, other diseases, conditions and disorders characterized by loss, damage or dysfunction of neurons including transplantation of neuron cells into individuals to treat individuals suspected of suffering from such diseases, conditions and disorders, any neurodegenerative disease of the eye, including photoreceptor loss in the retina in patients afflicted with macular degeneration, retinitis pigmentosa, glaucoma, and similar diseases, stroke, cerebral ischemia, head trauma, migraine, depression, peripheral neuropathy, pain, cerebral amyloid angiopathy, nootropic or cognition enhancement, multiple sclerosis, ocular angiogenesis, corneal injury, macular degeneration, tumor invasion, tumor growth, tumor metastasis, corneal scarring, scleritis, motor neuron and Lewy body disease, attention deficit disorder, migraine, narcolepsy, psychiatric disorders, panic disorders, social phobias, anxiety, psychoses, obsessive-compulsive disorders, obesity or eating disorders, body dysmorphic disorders, post-traumatic stress disorders, conditions associated with aggression, drug abuse treatment, or smoking secession, traumatic brain and spinal cord injury, and epilepsy.

Thus, the present invention also is directed to doses, dosage forms, formulations, compositions and/or devices comprising one or more copper antagonists, for example, one or more compounds of Formulae I and II and salts thereof, and one or more trientine active agents, including but not limited to, trientine, trientine dihydrochloride, trientine disuccinate, or other pharmaceutically acceptable salts thereof, trientine analogues and salts thereof, useful for the therapy of neurodegenerative diseases, disorders, and/or conditions in humans and other mammals and other disorders as disclosed herein. The use of these dosage forms, formulations compositions and/or devices of copper antagonism enables effective treatment of these conditions, through novel and improved formulations of the copper antagonists, for example, copper chelators, suitable for administration to humans and other mammals.

Evidence also supports the idea that diabetic patients who develop Alzheimer's have a modified permeability of the blood brain barrier. Firstly in the context of the proteomic analysis in Alzheimer's patients compared to matched controls show that the fragmentation pattern of serum albumin varied systematically between brain tissue from subjects with Alzheimer's and matched controls. It is normally thought that the blood brain barrier is impermeable to large proteins such as serum albumin; however; these findings indicate the presence of modified permeability in subjects with Alzheimer's disease. Secondly, in further studies it was demonstrated that cultured cortical neurons can process serum albumen in a reproducible manner to generate fragments including those that are similar to those observed in the brains of patients with Alzheimer's Disease. These findings relate to permeability of the blood brain barrier in Alzheimer's Disease and point to an underlying cause of this permeability. See Example 12. In other studies we have shown that accumulation of Cu²⁺ in the cardiovascular interstitial tissue leads to modified structure and function. Without intending or wishing to be bound by any particular theory or mechanism, accumulation of Cu²⁺ in the interstitial tissue of the cerebrovascular artery is identified as the process leading to increased permeability of the blood brain barrier in diabetic Alzheimer's Disease patients. The inventions described and claimed herein also include the use of the compounds provided or referenced for ameliorating or reversing permeability of the blood brain barrier. Modification of the blood brain barrier has utility, for example, in the treatment of neurodegenerative disorders, including those identified herein.

The invention provides, for example, dosage forms, formulations, devices and/or compositions containing one or more antagonists, for example, copper chelators, including one or more compounds of Formulae I and II and salts thereof, and trientine active agents, including but not limited to, trientine, trientine dihydrochloride or other pharmaceutically acceptable salts thereof, and salts thereof. The dosage forms, formulations, devices and/or compositions of the invention may be formulated to optimize bioavailability and to maintain plasma concentrations within the therapeutic range, including for extended periods, and results in increases in the time that plasma concentrations of the copper antagonist(s) remain within a desired therapeutic range at the site or sites of action. Controlled delivery preparations also optimize the drug concentration at the site of action and minimize periods of under and over medication, for example.

The dosage forms, formulated, devices and/or compositions of the invention may be formulated for periodic administration, including once daily administration, to provide low dose controlled and/or low dose long-lasting in vivo release of a copper antagonist, for example, a copper chelator for chelation of copper and excretion of chelated copper via the urine and/or to provide enhanced bioavailability of a copper antagonist, such as a copper chelator for chelation of copper and excretion of chelated copper via the urine.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of a copper antagonist.

Examples of dosage forms suitable for transdermal administration include, but are not limited, to transdermal patches, transdermal bandages, and the like.

Examples of dosage forms suitable for topical administration of the compounds and formulations of the invention are any lotion, stick, spray, ointment, paste, cream, gel, etc. whether applied directly to the skin or via an intermediary such as a pad, patch or the like.

Examples of dosage forms suitable for suppository administration of the compounds and formulations of the invention include any solid dosage form inserted into a bodily orifice particularly those inserted rectally, vaginally and urethrally.

Examples of dosage forms suitable for transmucosal delivery of the compounds and formulations of the invention include depositories solutions for enemas, pessaries, tampons, creams, gels, pastes, foams, nebulised solutions, powders and similar formulations containing in addition to the active ingredients such carriers as are known in the art to be appropriate.

Examples of dosage of forms suitable for injection of the compounds and formulations of the invention include delivery via bolus such as single or multiple administrations by intravenous injection, subcutaneous, subdermal, and intramuscular administration or oral administration.

Examples of dosage forms suitable for depot administration of the compounds and formulations of the invention include pellets or small cylinders of active agent or solid forms wherein the active agent is entrapped in a matrix of biodegradable polymers, microemulsions, liposomes or is microencapsulated.

Examples of infusion devices for compounds and formulations of the invention include infusion pumps containing one or more copper antagonists, for example one or more copper chelators, such as for example, one or more compounds of Formulae I and II and salts thereof, or trientine active agents, including but not limited to, trientine, trientine dihydrochloride, trintine disuccinate or other pharmaceutically acceptable salts thereof, at a desired amount for a desired number of doses or steady state administration, and include implantable drug pumps.

Examples of implantable infusion devices for compounds, and formulations of the invention include any solid form in which the active agent is encapsulated within or dispersed throughout a biodegradable polymer or synthetic, polymer such as silicone, silicone rubber, silastic or similar polymer.

Examples of dosage forms suitable for inhalation or insufflation of the compounds and formulations of the invention include compositions comprising solutions and/or suspensions in pharmaceutically acceptable, aqueous, or organic solvents, or mixture thereof and/or powders.

Examples of dosage forms suitable for buccal administration of the compounds and formulations of the invention include lozenges, tablets and the like, compositions comprising solutions and/or suspensions in pharmaceutically acceptable, aqueous, or organic solvents, or mixtures thereof and/or powders.

Examples of dosage forms suitable for sublingual administration of the compounds and formulations of the invention include lozenges, tablets and the like, compositions comprising solutions and/or suspensions in pharmaceutically acceptable, aqueous, or organic solvents, or mixtures thereof and/or powders.

Examples of dosage forms suitable for opthalmic administration of the compounds and formulations of the invention include inserts and/or compositions comprising solutions and/or suspensions in pharmaceutically acceptable, aqueous, or organic solvents.

Examples of controlled drug formulations useful for delivery of the compounds and formulations of the invention are found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. The individual monographs contain specific criteria for compliance with the test and the apparatus and test procedures to be used. Examples have been given, for example for the release of aspirin from Aspirin Extended-release Tablets (for example, see: Ansel, H. C., Allen, L. V. and Popovich, N. G., Pharmaceutical Dosage Forms and Drug Delivery Systems, 7th Ed., Lippincott 1999, p. 237). Modified-release tablets and capsules must meet the USP standard for uniformity as described for conventional dosage units. Uniformity of dosage units may be demonstrated by either of two methods, weight variation or content uniformity, as described in the USP. Further guidance concerning the analysis of extended release dosage forms has been provided by the F.D.A. (see Guidance for Industry. Extended release oral dosage forms:development, evaluation, and application of in vitro/in vivo correlations. Rockville, Md.:Center for Drug Evaluation and Research, Food and Drug Administration, 1997).

Further examples of dosage forms of the invention include, but are not limited to modified-release (MR) dosage forms including delayed-release (DR) forms; prolonged-action (PA) forms; controlled-release (CR) forms; extended-release (ER) forms; timed-release (TR) forms; and long-acting (LA) forms. For the most part, these terms are used to describe orally administered dosage forms, however these terms may be applicable to any of the dosage forms, formulations, compositions and/or devices described herein. These formulations effect delayed total drug release for some time after drug administration, and/or drug release in small aliquots intermittently after administration, and/or drug release slowly at a controlled rate governed by the delivery system, and/or drug release at a constant rate that does not vary, and/or drug release for a significantly longer period than usual formulations.

Modified-release dosage forms of the invention include dosage forms having drug release features based on time, course, and/or location which are designed to accomplish therapeutic or convenience objectives not offered by conventional or immediate-release forms. See, for example, Bogner, R. H. Bioavailability and bioequivalence of extended-release oral dosage forms. U.S. Pharmacist 22 (Suppl.):3-12 (1997); Scale-up of oral extended-release drug delivery systems:part I, an overview. Pharmaceutical Manufacturing 2:23-27 (1985). Extended-release dosage forms of the invention include, for example, as defined by The United States Food and Drug Administration (FDA), a dosage form that allows a reduction in dosing frequency to that presented by a conventional dosage form, e.g., a solution or an immediate-release dosage form. See, for example, Bogner, R. H. Bioavailability and bioequivalence of extended-release oral dosage forms. US Pharmacist 22 (Suppl.):3-12 (1997); Guidance for industry. Extended release oral dosage forms:development, evaluation, and application of the in vitro/in vivo correlations. Rockville, Md.:Center for Drug Evaluation and Research, Food and Drug Administration (1997). Repeat action dosage forms of the invention include, for example, forms that contain two single doses of medication, one for immediate release and the second for delayed release. Bi-layered tablets, for example, may be prepared with one layer of drug for immediate release with the second layer designed to release drug later as either a second dose or in an extended-release manner. Targeted-release dosage forms of the invention include, for example, formulations that facilitate drug release and which are directed towards isolating or concentrating a drug in a body region, tissue, or site for absorption or for drug action.

The invention in part provides dosage forms, formulations, devices and/or compositions and/or methods utilizing administration of dosage forms, formulations, devices and/or compositions incorporating one or more copper antagonists, for example one or more copper chelators, such as for example, one or more compounds of Formulae I or II and salts thereof, and trientine active agents, including but not limited to, trientine, trientine dihydrochloride, trientine disuccinate, or other pharmaceutically acceptable salts thereof, complexed with one or more suitable anions to yield complexes that are only slowly soluble in body fluids. One such example of modified release forms of one or more copper antagonists is produced by the incorporation of the active agent or agents into certain complexes such as those formed with the anions of various forms of tannic acid (for example, see:Merck Index 12th Ed., 9221). Dissolution of such complexes may depend, for example, on the pH of the environment. This slow dissolution rate provides for the extended release of the copper chelator. For example, salts of tannic acid, and/or tannates, provide for this quality, and are expected to possess utility for the treatment of conditions in which increased copper plays a role. Examples of equivalent products are provided by those having the tradename Rynatan (Wallace:see, for example, Madan, P. L., “Sustained release dosage forms,” U.S. Pharmacist 15:39-50 (1990); Ryna-12 S, which contains a mixture of mepyramine tannate with phenylephrine tannate, Martindale 33rd Ed., 2080.4).

Also included in the invention are coated beads, granules or microspheres containing one or more copper antagonists. Thus, the invention also provides a method to achieve modified release of one or more copper antagonists by incorporation of the drug into coated beads, granules, or microspheres. Such formulations of one or more copper antagonists have utility for the treatment of diseases in humans and other mammals in which a copper chelator, for example, trientine, is indicated. In such systems, the copper antagonist is distributed onto beads, pellets, granules or other particulate systems. Using conventional pan-coating or air-suspension coating techniques, a solution of the copper antagonist substance is placed onto small inert nonpareil seeds or beads made of sugar and starch or onto microcrystalline cellulose spheres. The nonpareil seeds are most often in the 425 to 850 micrometer range whereas the microcrystalline cellulose spheres are available ranging from 170 to 600 micrometers (see Ansel, H. C., Allen, L. V. and Popovich, N. G., Pharmaceutical Dosage Forms and Drug Delivery Systems, 7th Ed., Lippincott 1999, p. 232). The microcrystalline spheres are considered more durable during production than sugar-based cores (see:Celphere microcrystalline cellulose spheres. Philadelphia:FMC Corporation, 1996). Methods for manufacture of microspheres suitable for drug delivery have been described (see, for example, Arshady, R. Microspheres and microcapsules:a survey of manufacturing techniques. 1:suspension and cross-linking. Polymer Eng Sci 30:1746-1758 (1989); see also, Arshady, R., Micro-spheres and microcapsules:a survey of manufacturing techniques. 2:coacervation. Polymer Eng Sci 30:905-914 (1990); see also: Arshady R., Microspheres and micro-capsules:a survey of manufacturing techniques. 3:solvent evaporation. Polymer Eng Sci 30:915-924 (1990). In instances in which the copper antagonist dose is large, the starting granules of material may be composed of the copper antagonist itself. Some of these granules may remain uncoated to provide immediate copper antagonist release. Other granules (about two-thirds to three-quarters) receive varying coats of a lipid material such as beeswax, carnauba wax, glycerylmonostearate, cetyl alcohol, or a cellulose material such as ethylcellulose (infra). Subsequently, granules of different coating thickness are blended to achieve a mixture having the desired release characteristics. The coating material may be coloured with one or more dyes to distinguish granules or beads of different coating thickness (by depth of colour) and to provide distinctiveness to the product. When properly blended, the granules may be placed in capsules or tableted. Various coating systems are commercially available which are aqueous-based and which use ethylcellulose and plasticizer as the coating material (e.g., Aquacoat™ [FMC Corporation, Philadelphia] and Surerelease™ [Colorcon]; Aquacoat aqueous polymeric dispersion. Philadelphia:FMC Corporation, 1991; Surerelease aqueous controlled release coating system. West Point, Pa.:Colorcon, 1990; Butler, J., Cumming, I, Brown, J. et al., A novel multiunit controlled-release system, Pharm Tech 22:122-138 (1998); Yazici, E., Oner, L., Kas, H. S. & Hincal, A. A., Phenyloin sodium microspheres: bench scale formulation, process characterization and release kinetics, Pharmaceut Dev Technol 1:175-183 (1996)). Aqueous-based coating systems eliminate the hazards and environmental concerns associated with organic solvent-based systems. Aqueous and organic solvent-based coating methods have been compared (see, for example, Hogan, J. E. Aqueous versus organic solvent coating. Int J Pharm Tech Prod Manufacture 3:17-20 (1982)). The variation in the thickness of the coats and in the type of coating materials used affects the rate at which the body fluids are capable of penetrating the coating to dissolve the copper antagonist. Generally, the thicker the coat, the more resistant to penetration and the more delayed will be copper antagonist release and dissolution. Typically, the coated beads are about 1 mm in diameter. They are usually combined to have three or four release groups among the more than 100 beads contained in the dosing unit (see Madan, P. L. Sustained release dosage forms. U.S. Pharmacist 15:39-50 (1990)). This provides the different desired sustained or extended release rates and the targeting of the coated beads to the desired segments of the gastrointestinal tract. One example of this type of dosage form is the Spansule™ (SmithKline Beecham Corporation, U.K.). Examples of film-forming polymers which can be used in water-insoluble release-slowing intermediate layer(s) (to be applied to a pellet, spheroid or tablet core) include ethylcellulose, polyvinyl acetate, Eudragit® RS, Eudragit® RL, etc. (Each of Eudragit® RS and Eudragit® RL is an ammonio methacrylate copolymer. The release rate can be controlled not only by incorporating therein suitable water-soluble pore formers, such as lactose, mannitol, sorbitol, etc., but also by the thickness of the coating layer applied. Multi tablets may be formulated which include small spheroid-shaped compressed minitablets that may have a diameter of between 3 to 4 mm and can be placed in gelatin capsule shell to provide the desired pattern of copper chelator release. Each capsule may contain 8-10 minitablets, some uncoated for immediate release and others coated for extended release of the copper chelator of the invention.

A number of methods may be employed to generate modified-release dosage forms of one or more copper antagonists suitable for oral administration to humans and other mammals. Two basic mechanisms are available to achieve modified release drug delivery. These are altered dissolution or diffusion of drugs and excipients. Within this context, for example, four processes may be employed, either simultaneously or consecutively. These are as follows:(i) hydration of the device (e.g., swelling of the matrix); (ii) diffusion of water into the device; (iii) controlled or delayed dissolution of the drug; and (iv) controlled or delayed diffusion of dissolved or solubilized drug out of the device.

For orally administered dosage forms of the compounds and formulations of the invention, extended antagonist action, for example, copper chelator action, may be achieved by affecting the rate at which the copper antagonist is released from the dosage form and/or by slowing the transit time of the dosage form through the gastrointestinal tract (see Bogner, R. H. Bioavailability and bioequivalence of extended-release oral dosage forms. U.S. Pharmacist 22 (Suppl.):3-12 (1997)). The rate of drug release from solid dosage forms may be modified by the technologies described below which, in general, are based on the following:1) modifying drug dissolution by controlling access of biologic fluids to the drug through the use of barrier coatings; 2) controlling drug diffusion rates from dosage forms; and 3) chemically reacting or interacting between the drug substance or its pharmaceutical barrier and site-specific biological fluids. Systems by which these objectives are achieved are also provided herein. In one approach, employing digestion as the release mechanism, the copper antagonist is either coated or entrapped in a substance that is slowly digested or dispersed into the intestinal tract. The rate of availability of the copper antagonist is a function of the rate of digestion of the dispersible material. Therefore, the release rate, and thus the effectiveness of the copper antagonist, varies from subject to subject depending upon the ability of the subject to digest the material.

A further form of slow release dosage form of the compounds and formulations of the invention is any suitable osmotic system where semipermeable membranes of for example cellulose acetate, cellulose acetate butyrate, cellulose acetate propionate, is used to control the release of copper chelator. These can be coated with aqueous dispersions of enteric lacquers without changing release rate. An example of such an osmotic system is an osmotic pump device, an example of which is the Oros™ device developed by Alza Inc. (U.S.A.). This system comprises a core tablet surrounded by a semi-permeable membrane coating having a 0.4 mm diameter hole produced by a laser beam. The core tablet has two layers, one containing the drug (the “active” layer) and the other containing a polymeric osmotic agent (the “push” layer). The core layer consists of active drug, filler, a viscosity modulator, and a solubilizer. The system operates on the principle of osmotic pressure. This system is suitable for delivery of a wide range of copper antagonists, including the compounds of Formulae I and II, and trientine active agents, or salts of any of them. The coating technology is straightforward, and release is zero-order. When the tablet is swallowed, the semi-permeable membrane permits aqueous fluid to enter from the stomach into the core tablet, dissolving or suspending the copper antagonist. As pressure increases in the osmotic layer, it forces or pumps the copper antagonist solution out of the delivery orifice on the side of the tablet. Only the copper antagonist solution (not the undissolved copper antagonist) is capable of passing through the hole in the tablet. The system is designed such that only a few drops of water are drawn into the tablet each hour. The rate of inflow of aqueous fluid and the function of the tablet depends on the existence of an osmotic gradient between the contents of the bi-layer and the fluid in the gastrointestinal tract. Copper antagonist delivery is essentially constant as long as the osmotic gradient remains unchanged. The copper antagonist release rate may be altered by changing the surface area, the thickness or composition of the membrane, and/or by changing the diameter of the copper antagonist release orifice. The copper antagonist-release rate is not affected by gastrointestinal acidity, alkalinity, fed conditions, or gut motility. The biologically inert components of the tablet remain intact during gut transit and are eliminated in the feces as an insoluble shell. Other examples of the application of this technology are provided by Glucotrol XL Extended Release Tablets (Pfizer Inc.) and Procardia XL Extended Release Tablets (Pfizer Inc.; see, Martindale 33rd Ed., p. 2051.3).

The invention also provides devices for compounds and formulations of the invention that utilize monolithic matrices including, for example, slowly eroding or hydrophilic polymer matrices, in which one or more copper antagonists is compressed or embedded.

Monolithic matrix devices comprising compounds and formulations of the invention include those formed using either of the following systems, for example:(I), copper antagonist dispersed in a soluble matrix, which become increasingly available as the matrix dissolves or swells; examples include hydrophilic colloid matrices, such as hydroxypropylcellulose (BP) or hydroxypropyl cellulose (USP); hydroxypropyl methylcellulose (HPMC; BP, USP); methylcellulose (MC; BP, USP); calcium carboxymethylcellulose (Calcium CMC; BP, USP); acrylic acid polymer or carboxy polymethylene (Carbopol) or Carbomer (BP, USP); or linear glycuronan polymers such as alginic acid (BP, USP), for example those formulated into microparticles from alginic acid (alginate)-gelatin hydrocolloid coacervate systems, or those in which liposomes have been encapsulated by coatings of alginic acid with poly-L-lysine membranes. Copper antagonist release occurs as the polymer swells, forming a matrix layer that controls the diffusion of aqueous fluid into the core and thus the rate of diffusion of copper antagonist from the system. In such systems, the rate of copper antagonist release depends upon the tortuous nature of the channels within the gel, and the viscosity of the entrapped fluid, such that different release kinetics can be achieved, for example, zero-order, or first-order combined with pulsatile release. Where such gels are not cross-linked, there is a weaker, non-permanent association between the polymer chains, which relies on secondary bonding. With such devices, high loading of the copper antagonist is achievable, and effective blending is frequent. Devices may contain 20-80% of copper antagonist (w/w), along with gel modifiers that can enhance copper antagonist diffusion; examples of such modifiers include sugars that can enhance the rate of hydration, ions that can influence the content of cross-links, and pH buffers that affect the level of polymer ionization. Hydrophilic matrix devices of the invention may also contain one or more of pH buffers, surfactants, counter-ions, lubricants such as magnesium stearate (BP, USP) and a glidant such as colloidal silicon dioxide (USP; colloidal anhydrous silica, BP) in addition to copper chelator and hydrophilic matrix; (II) copper antagonist particles are dissolved in an insoluble matrix, from which copper antagonist becomes available as solvent enters the matrix, often through channels, and dissolves the copper antagonist particles. Examples include systems formed with a lipid matrix, or insoluble polymer matrix, including preparations formed from Carnauba wax (BP; USP); medium-chain triglyceride such as fractionated coconut oil (BP) or triglycerida saturata media (PhEur); or cellulose ethyl ether or ethylcellulose (BP, USP). Lipid matrices are simple and easy to manufacture, and incorporate the following blend of powdered components:lipids (20-40% hydrophobic solids w/w) which remain intact during the release process; copper antagonist, e.g., copper chelator; channeling agent, such as sodium chloride or sugars, which leaches from the formulation, forming aqueous micro-channels (capillaries) through which solvent enters, and through which copper antagonist is released. In the alternative system, which employs an insoluble polymer matrix, the copper antagonist is embedded in an inert insoluble polymer and is released by leaching of aqueous fluid, which diffuses into the core of the device through capillaries formed between particles, and from which copper antagonist diffuses out of the device. The rate of release is controlled by the degree of compression, particle size, and the nature and relative content (w/w) of excipients. An example of such a device is that of Ferrous Gradumet (Martindale 33rd Ed., 1360.3). A further example of a suitable insoluble matrix is an inert plastic matrix. By this method, copper antagonist is granulated with an inert plastic material such as polyethylene, polyvinyl acetate, or polymethacrylate, and the granulated mixture is then compressed into tablets. Once ingested, the copper antagonist is slowly released from the inert plastic matrix by diffusion (see, for example, Bodmeier, R. & Paeratakul, O., “Drug release from laminated polymeric films prepared from aqueous latexes,” J Pharm Sci 79:32-26 (1990); Laghoueg, N., et al., “Oral polymer-drug devices with a core and an erodable shell for constant drug delivery,” Int J Pharm 50:133-139(1989); Buckton, G., et al., “The influence of surfactants on drug release from acrylic matrices. Int J Pharm 74:153-158 (1991)). The compression of the tablet creates the matrix or plastic form that retains its shape during the leaching of the copper antagonist and through its passage through the gastrointestinal tract. An immediate-release portion of copper antagonist may be compressed onto the surface of the tablet. The inert tablet matrix, expended of copper antagonist, is excreted with the feces. An example of a successful dosage form of this type is Gradumet (Abbott; see, for example, Ferro-Gradumet, Martindale 33rd Ed., p. 1860.4).

Further examples of monolithic matrix devices of the invention have compounds and formulations of the invention incorporated in pendent attachments to a polymer matrix (see, for example, Scholsky, K. M. and Fitch, R. M., Controlled release of pendant bioactive materials from acrylic polymer colloids. J Controlled Release 3:87-108 (1986)). In these devices, copper antagonists, e.g., copper chelators, are attached by means of an ester linkage to poly(acrylate) ester latex particles prepared by aqueous emulsion polymerization.

Yet further examples of monolithic matrix devices of the invention incorporate dosage forms of the compounds and formulations of the invention in which the copper antagonist is bound to a biocompatible polymer by a labile chemical bond, e.g., polyanhydrides prepared from a substituted anhydride (itself prepared by reacting an acid chloride with the drug: methacryloyl chloride and the sodium salt of methoxy benzoic acid) have been used to form a matrix with a second polymer (Eudragit RL) which releases drug on hydrolysis in gastric fluid (see:Chafi, N., Montheard, J. P. & Vergnaud, J. M. Release of 2-aminothiazole from polymeric carriers. Int J Pharm 67:265-274 (1992)).

In formulating a successful hydrophilic matrix system for the compounds and formulations of the invention, the polymer selected for use must form a gelatinous layer rapidly enough to protect the inner core of the tablet from disintegrating too rapidly after ingestion. As the proportion of polymer is increased in a formulation so is the viscosity of the gel formed with a resulting decrease in the rate of copper antagonist diffusion and release (see Formulating for controlled release with Methocel Premium cellulose ethers. Midland, Mich.:Dow Chemical Company, 1995). In general, 20% (w/w) of HPMC results in satisfactory rates of drug release for an extended-release tablet formulation. However, as with all formulations, consideration must be given to the possible effects of other formulation ingredients such as fillers, tablet binders, and disintegrants. An example of a proprietary product formulated using a hydrophilic matrix base of HPMC for extended drug release is that of Oramorph SR Tablets (Roxane; see Martindale 33rd Ed., p. 2014.4).

Two-layered tablets can be manufactured containing one or more of the compounds and formulations of the invention, with one layer containing the uncombined copper antagonist for immediate release and the other layer having the copper antagonist imbedded in a hydrophilic matrix for extended-release. Three-layered tablets may also be similarly prepared, with both outer layers containing the copper antagonist for immediate release. Some commercial tablets are prepared with an inner core containing the extended-release portion of drug and an outer shell enclosing the core and containing drug for immediate release.

The invention also provides forming a complex between the compounds and formulations of the invention and an ion exchange resin, whereupon the complex may be tableted, encapsulated or suspended in an aqueous vehicle. Release of the copper antagonist is dependent on the local pH and electrolyte concentration such that the choice of ion exchange resin may be made so as to preferentially release the copper antagonist in a given region of the alimentary canal. Delivery devices incorporating such a complex are also provided. For example, a modified release dosage form of copper antagonist can be produced by the incorporation of copper antagonist into complexes with an anion-exchange resin. Solutions of copper antagonist may be passed through columns containing an ion-exchange resin to form a complex by the replacement of H₃O⁺ ions. The resin-trientine complex is then washed and may be tableted, encapsulated, or suspended in an aqueous vehicle. The release of the copper antagonist is dependent on the pH and the electrolyte concentration in the gastrointestinal fluid. Release is greater in the acidity of the stomach than in the less acidic environment of the small intestine. Alternative examples of this type of extended release preparation are provided by hydrocodone polistirex and chorpheniramine polistirex suspension (Medeva; Tussionex Pennkinetic Extended Release Suspension, see:Martindale 33rd Ed., p. 2145.2) and by phentermine resin capsules (Pharmanex; Ionamin Capsules see:Martindale 33rd Ed., p. 1916.1). Such resin-copper antagonist systems can additionally incorporate polymer barrier coating and bead technologies in addition to the ion-exchange mechanism. The initial dose comes from an uncoated portion, and the remainder from the coated beads, wherein release may be extended over a 12-hour period by ion exchange. The copper antagonist containing particles are minute, and may also be suspended to produce a liquid with extended-release characteristics, as well as solid dosage forms. Such preparations may also be suitable for administration, for example in depot preparations suitable for intramuscular injection.

The invention also provides a method to produce modified release preparations of one or more copper antagonists, for example, one or more copper chelators, by microencapsulation. Microencapsulation is a process by which solids, liquids, or even gasses may be encapsulated into microscopic size particles through the formation of thin coatings of “wall” material around the substance being encapsulated such as disclosed in U.S. Pat. Nos. 3,488,418; 3,391,416 and 3,155,590. Gelatin (BP, USP) is commonly employed as a wall-forming material in microencapsulated preparations, but synthetic polymers such as polyvinyl alcohol (USP), ethylcellulose (BP, USP), polyvinyl chloride, and other materials may also be used (see, for example, Zentner, G. M., Rork, G. S., and Himmelstein, K. J., Osmotic flow through controlled porosity films:an approach to delivery of water soluble compounds, J Controlled Release 2:217-229 (1985); Fites, A. L., Banker, G. S., and Smolen, V. F., Controlled drug release through polymeric films, J Pharm Sci 59:610-613 (1970); Samuelov, Y., Donbrow, M., and Friedman, M., Sustained release of drugs from ethylcellulose-polyethylene glycol films and kinetics of drug release, J Pharm Sci 68:325-329 (1979)).

Encapsulation begins with the dissolving of the prospective wall material, say gelatin, in water. One or more copper antagonist, for example, one or more copper chelators, is then added and the two-phase mixture is thoroughly stirred. With the material to be encapsulated broken up to the desired particle size, a solution of a second material is added. This additive material, for example, acacia, is chosen to have the ability to concentrate the gelatin (polymer) into tiny liquid droplets. These droplets (the coacervate) then form a film or coat around the particles of the solid copper chelator as a consequence of the extremely low interfacial tension of the residual water or solvent in the wall material so that a continuous, tight, film-coating remains on the particle (see Ansel, H. C., Allen, L. V., and Popovich, N. G., Pharmaceutical Dosage Forms and Drug Delivery Systems, 7th Ed., Lippincott 1999, p. 233). The final dry microcapsules are free flowing, discrete particles of coated material. Of the total particle weight, the wall material usually represents between 2 and 20% (w/w). The coated particles are then admixed with tableting excipients and formed into dosage-sized tablets. Different rates of copper antagonist release may be obtained by changing the core-to-wall ratio, the polymer used for the coating, or the method of microencapsulation (for example, see:Yazici, E., Oner, L., Kas, H. S. & Hincal, A. A. Phenyloin sodium microspheres:bench scale formulation, process characterization and release kinetics. Pharmaceut Dev Technol 1996; 1:175-183).

One of the advantages of microencapsulation is that the administered dose of one or more copper antagonists, for example, one or more copper chelators, is subdivided into small units that are spread over a large area of the gastrointestinal tract, which may enhance absorption by diminishing localized copper chelator concentrations (see Yazici et al., supra). An example of a drug that is commercially available in a microencapsulated extended-release dosage form is potassium chloride (Micro-K Exten-caps, Wyeth-Ayerst, Martindale 33rd Ed., p 1968.1). Other useful approaches include those in which the copper antagonist is incorporated into polymeric colloidal particles or microencapsulates (microparticles, microspheres or nanoparticles) in the form or reservoir and matrix devices (see:Douglas, S. J., et al., “Nanoparticles in drug delivery,” C. R. C. Crit Rev Therap Drug Carrier Syst 3:233-261 (1987); Oppenheim, R. C., “Solid colloidal drug delivery systems:nanoparticles,” Int J Pharm 8:217-234 (1981); Higuchi, T., “Mechanism of sustained action medication:theoretical analysis of rate of release of solid drugs dispersed in solid matrices,” J Pharm Sci 52:1145-1149 (1963)).

The invention also includes repeat action tablets containing one or more copper antagonists, for example, one or more copper chelators. These are prepared so that an initial-dose of the copper chelator is released immediately followed later by a second dose. The tablets may be prepared with the immediate-release dose in the tablet's outer shell or coating with the second dose in the tablet's inner core, separated by a slowly permeable barrier coating. In general, the copper antagonist from the inner core is exposed to body fluids and released 4 to 6 hours after administration. An example of this type of product is proved by Repetabs (Schering Inc.). Repeat action dosage forms are suitable for the administration of one or more copper antagonists for the indications noted herein.

The invention also includes delayed-release oral dosage forms containing one or more copper antagonists, for example, one or more copper chelators. The release of one or more copper antagonist, for example, one or more copper chelators, from an oral dosage form can be intentionally delayed until it reaches the intestine at least in part by way of, for example, enteric coating. Enteric coatings by themselves are not an efficient method for the delivery of copper antagonists because of the inability of such coating systems to provide or achieve a sustained therapeutic effect after release onset. Enteric coats are designed to dissolve or break down in an alkaline environment. The presence of food may increase the pH of the stomach. Therefore, the concurrent administration of enteric-coated copper antagonists with food or the presence of food in the stomach may lead to dose dumping and unwanted secondary effects. Furthermore, in the event of gastrointestinal side-effects, it would be desirable to have a copper chelator form that is capable of providing the controlled delivery of copper antagonists in a predictable manner over a long period of time.

Enteric coatings have application in the present invention when combined or incorporated with one or more of the other dose delivery formulations or devices described herein. This form of delivery conveys the advantage of minimizing the gastric irritation that may be caused in some subjects by copper antagonist such as, for example, trientine. The enteric coating may be time-dependent, pH-dependent where it breaks down in the less acidic environment of the intestine and erodes by moisture over time during gastrointestinal transit, or enzyme-dependent where it deteriorates due to the hydrolysis-catalyzing action of intestinal enzymes (see, for example, Muhammad, N. A., et al., “Modifying the release properties of Eudragit L30D,” Drug Dev Ind Pharm., 17:2497-2509 (1991)). Among the many agents used to enteric coat tablets and capsules known to those skilled in the art are fats including triglycerides, fatty acids, waxes, shellac, and cellulose acetate phthalate although further examples of enteric coated preparations can be found in the USP.

The invention also provides devices incorporating one or more copper antagonists, for example, one or more copper chelators, in a membrane-control system. Such devices comprise a rate-controlling membrane enclosing a copper antagonist reservoir. Following oral administration the membrane gradually becomes permeable to aqueous fluids, but does not erode or swell. The copper antagonist reservoir may be composed of a conventional tablet, or a microparticle pellet containing multiple units that do not swell following contact with aqueous fluids. The cores dissolve without modifying their internal osmotic pressure, thereby avoiding the risk of membrane rupture, and typically comprise 60:40 mixtures of lactulose: microcrystalline cellulose (w/w). Copper antagonist(s) is(are) released through a two-phase process, comprising diffusion of aqueous fluids into the matrix, followed by diffusion of the copper antagonist out of the matrix. Multiple-unit membrane-controlled systems typically comprise more than one discrete unit. They can contain discrete spherical beads individually coated with rate-controlling membrane and may be encapsulated in a hard gelatin shell (examples of such preparations include Contac 400; Martindale 33rd Ed., 1790.1 and Feospan; Martindale 33rd Ed., p. 1859.4). Alternatively, multiple-unit membrane-controlled systems may be compressed into a tablet (for example, Suscard; Martindale 33rd Ed., p. 2115.1). Alternative implementations of this technology include devices in which the copper antagonist is coated around inert sugar spheres, and devices prepared by extrusion spheronization employing a conventional matrix system. Advantages of such systems include the more consistent gastro-intestinal transit rate achieved by multiple-unit systems, and the fact that such systems infrequently suffer from catastrophic dose dumping. They are also ideal for the delivery of more than one drug at a time.

An example of a sustained release dosage form of one or more compounds and formulations of the invention is a matrix formation, such a matrix formation taking the form of film coated spheroids containing as active ingredient one or more copper antagonists, for example, one or more copper chelators and a non water soluble spheronising agent. The term “spheroid” is known in the pharmaceutical art and means spherical granules having a diameter usually of between 0.01 mm and 4 mm. The spheronising agent may be any pharmaceutically acceptable material that, together with the copper antagonist, can be spheronised to form spheroids. Microcrystalline cellulose is preferred. Suitable microcrystalline cellulose includes, for example, the material sold as Avicel PH 101 (Trade Mark, FMC Corporation). The film-coated spheroids may contain between 70% and 99% (by wt), especially between 80% and 95% (by wt), of the spheronising agent, especially microcrystalline cellulose. In addition to the active ingredient and spheronising agent, the spheroids may also contain a binder. Suitable binders, such as low viscosity, water soluable polymers, will be well known to those skilled in the pharmaceutical art. A suitable binder is, in particular polyvinylpyrrolidone in various degrees of polymerization. However, water-soluble hydroxy lower alkyl celluloses, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate copolymer, or ethyl cellulose. Other thickening agents or binders include:the lipid type, among which are vegetable oils (cotton seed, sesame and groundnut oils) and derivatives of these oils (hydrogenated oils such as hydrogenated castor oil, glycerol behenate, the waxy type such as natural carnauba wax or natural beeswax, synthetic waxes such as cetyl ester waxes, the amphiphilic type such as polymers of ethylene oxide (polyoxyethylene glycol of high molecular weight between 4000 and 100000) or propylene and ethylene oxide copolymers (poloxamers), the cellulosic type (semisynthetic derivatives of cellulose, hydroxypropylmethylcellulose, hydroxypropylcellulose, hydroxymethylcellulose, of high molecular weight and high viscosity, gum) or any other polysaccharide such as alginic acid, the polymeric type such as acrylic acid polymers (such as carbomers), and the mineral type such as colloidal silica and bentonite.

Suitable diluents for the copper antagonist(s) in the pellets, spheroids or core are, e.g., microcrystalline cellulose, lactose, dicalcium phosphate, calcium carbonate, calcium sulphate, sucrose, dextrates, dextrin, dextrose, dicalcium phosphate dihydrate, kaolin, magnesium carbonate, magnesium oxide, maltodextrin, cellulose, microcrystalline cellulose, sorbitol, starches, pregelatinized starch, talc, tricalcium phosphate and lactose. Suitable lubricants are e.g., magnesium stearate and sodium stearyl fumarate. Suitable binding agents include, e.g., hydroxypropyl methylcellulose, polyvidone, and methylcellulose.

Suitable binders that may be included are:gum arabic, gum tragacanth, guar gum, alginic acid, sodium alginate, sodium carboxymethylcellulose, dextrin, gelatin, hydroxyethylcellulose, hydroxypropylcellulose, liquid glucose, magnesium and aluminum. Suitable disintegrating agents are starch, sodium starch glycolate, crospovidone and croscarmalose sodium. Suitable surface active are Poloxamer 188®, polysorbate 80 and sodium lauryl sulfate. Suitable flow aids are talc colloidal anhydrous silica. Suitable lubricants that may be used are glidants (such as anhydrous silicate, magnesium trisilicate, magnesium silicate, cellulose, starch, talc or tricalcium phosphate) or alternatively antifriction agents (such as calcium stearate, hydrogenated vegetable oils, paraffin, magnesium stearate, polyethylene glycol, sodium benzoate, sodium lauryl sulphate, fumaric acid, stearic acid or zinc stearate and talc). Suitable water-soluble polymers are PEG with molecular weights in the range 1000 to 6000.

Delayed release of the composition or formulation of the invention may be achieved through the use of a tablet, pellet, spheroid or core itself, which besides having a filler and binder, other ancillary substances, in particular lubricants and nonstick agents, and disintegrants. Examples of lubricants and nonstick agents are higher fatty acids and their alkali metal and alkaline-earth-metal salts, such as calcium stearate. Suitable disintegrants are, in particular, chemically inert agents, for example, cross-linked polyvinylpyrrolidone, cross-linked sodium carboxymethylcelluloses, and sodium starch glycolate.

Yet further embodiments of the invention include formulations of one or more copper antagonists, for example, one or more copper chelators, incorporated into transdermal drug delivery systems, such as those described in:Transdermal Drug Delivery Systems, Chapter 10. In:Ansel, H. C., Allen, L. V. and Popovich, N. G. Pharmaceutical Dosage Forms and Drug Delivery Systems, 7th Ed., Lippincott 1999, pp. 263-278). Transdermal drug delivery systems facilitate the passage of therapeutic quantities of drug substances through the skin and into the systemic circulation to exert systemic effects, as originally described (see Stoughton, R. D. Percutaneous absorption, Toxicol Appl Pharmacol 7:1-8 (1965)). Evidence of percutaneous drug absorption may be found through measurable blood levels of the drug, detectable excretion of the drug and/or its metabolites in the urine, and through the clinical response of the subject to its administration. For transdermal drug delivery, it is considered ideal if the drug penetrates through the skin to the underlying blood supply without drug build up in the dermal layers (Black, C. D., “Transdermal drug delivery systems,” U.S. Pharm 1:49 (1982)). Formulations of drugs suitable for trans-dermal delivery are known to those skilled in the art, and are described in references such as Ansel et al., (supra). Methods known to enhance the delivery of drugs by the percutaneous route include chemical skin penetration enhancers, which increase skin permeability by reversibly damaging or otherwise altering the physicochemical nature of the stratum corneum to decrease its resistance to drug diffusion (see Shah, V., Peck, C. C., and Williams, R. L., Skin penetration enhancement:clinical pharmacological and regulatory considerations, In:Walters, K. A. and Hadgraft, J. (Eds.) Pharmaceutical skin penetration enhancement. New York:Dekker, 1993). Among effective alterations are increased hydration of the stratum corneum and/or a change in the structure of the lipids and lipoproteins in the intercellular channels brought about through solvent action or denaturation (see Walters K. A., “Percutaneous absorption and transdermal therapy,” Pharm Tech 10:30-42 (1986)). Skin penetration enhancers suitable for formulation with copper antagonist in transdermal drug delivery systems may be chosen from the following list:acetone, laurocapram, dimethylacetamide, dimethylformamide, dimethylsulphoxide, ethanol, oleic acid, polyethylene glycol, propylene glycol and sodium lauryl sulphate. Further skin penetration enhancers may be found in publications known to those skilled in the art (see, for example, Osborne, D. W., & Henke, J. J., “Skin penetration enhancers cited in the technical literature,” Pharm Tech 21:50-66 (1997); Rolf, D., “Chemical and physical methods of enhancing transdermal drug delivery,” Pharm Tech 12:130-139 (1988)).

In addition to chemical means, there are physical methods that enhance transdermal drug delivery and penetration of the compounds and formulations of the invention. These include iontophoresis and sonophoresis. Iontophoresis involves the delivery of charged chemical compounds across the skin membrane using an applied electrical field. Such methods have proven suitable for delivery of a number of drugs. Accordingly, another embodiment of the invention comprises one or more copper antagonists, for example, one or more copper chelators, formulated in such a manner suitable for administration by iontophoresis or sonophoresis. Formulations suitable for administration by iontophoresis or sonophoresis may be in the form of gels, creams, or lotions. Transdermal delivery, methods or formulations of the invention, may utilize, among others, monolithic delivery systems, drug-impregnated adhesive delivery systems (e.g., the Latitude™ drug-in-adhesive system from 3M), active transport devices and membrane-controlled systems. Monolithic systems of the invention incorporate a copper antagonist matrix, comprising a polymeric material in which the copper antagonist is dispersed between backing and frontal layers. Drug impregnated adhesive delivery systems comprise an adhesive polymer in which one or more compounds and formulations of the invention and any excipients are incorporated into the adhesive polymer. Active transport devices incorporate a copper antagonist reservoir, often in liquid or gel form, a membrane that may be rate controlling, and a driving force to propel the copper chelator across the membrane. Membrane-controlled transdermal systems of the invention comprise a copper antagonist reservoir, often in liquid or gel form, a membrane that may be rate controlling and backing, adhesive and/or protecting layers. Transdermal delivery dosage forms of the invention include those which substitute the copper antagonist, for the diclofenic or other pharmaceutically acceptable salt thereof referred to in the transdermal delivery systems disclosed in, by way of example, U.S. Pat. Nos. 6,193,996, and 6,262,121.

Formulations and/or compositions for topical administration of one or more compounds and formulations of the invention ingredient can be prepared as an admixture or other pharmaceutical formulation to be applied in a wide variety of ways including, but are not limited to, lotions, creams gels, sticks, sprays, ointments and pastes. These product types may comprise several types of formulations including, but not limited to solutions, emulsions, gels, solids, and liposomes. If the topical composition of the invention is formulated as an aerosol and applied to the skin as a spray-on, a propellant may be added to a solution composition. Suitable propellants as used in the art can be utilized. By way of example of topical administration of an active agent, reference is made to U.S. Pat. Nos. 5,602,125, 6,426,362 and 6,420,411.

Also included in the dosage forms in accordance with the present invention are any variants of the oral dosage forms that are adapted for suppository or other parenteral use. When rectally administered in the form of suppositories, for example, these compositions may be prepared by mixing one or more compounds and formulations of the invention with a suitable non-irritating excipient, such as cocoa butter, synthetic glyceride esters or polyethylene glycols, which are solid at ordinary temperatures, but liquidity and/or dissolve in the rectal cavity to release the copper chelator. Suppositories are generally solid dosage forms intended for insertion into body orifices including rectal, vaginal and occasionally urethrally and can be long acting or slow release. Suppositories include a base that can include, but is not limited to, materials such as alginic acid, which will prolong the release of the pharmaceutically acceptable active ingredient over several hours (5-7). Such bases can be characterized into two main categories and a third miscellaneous group:1) fatty or oleaginous bases, 2) water-soluble or water-miscible bases and 3) miscellaneous bases, generally combinations of lipophilic and hydrophilic substances. Fatty or oleaginous bases include hydrogenated fatty acids of vegetable oils such as palm kernel oil and cottonseed oil, fat-based compound containing compounds of glycerin with the higher molecular weight fatty acids such as palmitic and stearic acids, cocoa butter is also used where phenol and chloral hydrate lower the melting point of cocoa butter when incorporated, solidifying agents like cetyl esters wax (about 20%) or beeswax (about 4%) may be added to maintain a solid suppository. Other bases include other commercial products such as Fattibase (triglycerides from palm, palm kernel and coconut oils with self-emulsifying glycerol monostearate and poloxyl stearate), Wecobee and Witepsol bases. Water-soluble bases are generally glycerinated gelatin and water-miscible bases are generally polyethylene glycols. The miscellaneous bases include mixtures of the oleaginous and water-soluble or water-miscible materials. An example of such a base in this group is polyoxyl 40 stearate and polyoxyethylene diols and the free glycols.

Transmucosal administration of the compounds and formulations of the invention may utilize any mucosal membrane but commonly utilizes the nasal, buccal, vaginal and rectal tissues.

Formulations suitable for nasal administration of the compounds and formulations of the invention may be administered in a liquid form, for example, nasal spray, nasal drops, or by aerosol administration by nebulizer, including aqueous or oily solutions of the copper chelator. Formulations for nasal administration, wherein the carrier is a solid, include a coarse powder having a particle size, for example, of less than about 100 microns, preferably less than about 50 microns, which is administered in the manner in which snuff is taken, i.e., by rapid inhalation through the nasal passage from a container of the powder held close up to the nose. Compositions in solution may be nebulized by the use of inert gases and such nebulized solutions may be breathed directly from the nebulizing device or the nebulizing device may be attached to a facemask, tent or intermittent positive-pressure breathing machine. Solutions, suspensions or powder compositions of the copper chelator may be administered orally or nasally from devices that deliver the formulation in an appropriate manner. Formulations of the invention may be prepared as aqueous solutions for example in saline, solutions employing benzyl alcohol or other suitable preservatives, absorption promoters to enhance bio-availability, fluorocarbons, and/or other solubilising or dispersing agents known in the art.

The invention provides extended-release formulations containing one or more copper antagonists, for example, one or more copper chelators, for parenteral administration. Extended rates of copper antagonist action following injection may be achieved in a number of ways, including the following:crystal or amorphous copper antagonist forms having prolonged dissolution characteristics; slowly dissolving chemical complexes of the copper antagonist entity; solutions or suspensions of copper antagonist in slowly absorbed carriers or vehicles (as oleaginous); increased particle size of copper antagonist in suspension; or, by injection of slowly eroding microspheres of copper antagonist (for example, see:Friess, W., Lee, G. and Groves, M. J. Insoluble collagen matrices for prolonged delivery of proteins. Pharmaceut Dev Technol 1:185-193 (1996)). The duration of action of the various forms of insulin for example is based in part on its physical form (amorphous or crystalline), complex formation with added agents, and its dosage form (solution of suspension).

The copper antagonist of the invention can be formulated into a pharmaceutical composition suitable for administration to a patient. The composition can be prepared according to conventional methods by dissolving or suspending an amount of the copper antagonist ingredient in a diluent. The amount is from between 0.1 mg to 1000 mg per ml of diluent of the copper antagonist. In some embodiments, dosage forms of 100 mg and 200 mg of a copper antagonist, for example, a copper chelator, are provided. The copper antagonist can be provided and administered in forms suitable for once-a-day dosing. An acetate, phosphate, citrate or glutamate buffer may be added allowing a pH of the final composition to be from about 5.0 to about 9.5; optionally a carbohydrate or polyhydric alcohol tonicifier and, a preservative selected from the group consisting of m-cresol, benzyl alcohol, methyl, ethyl, propyl and butyl parabens and phenol may also be added. A sufficient amount of water for injection is used to obtain the desired concentration of solution. Additional tonicifying agents such as sodium chloride, as well as other excipients, may also be present, if desired. Such excipients, however, must maintain the overall tonicity of the copper antagonist composition, as parenteral formulations must be isotonic or substantially isotonic otherwise significant irritation and pain would occur at the site of administration.

The terms buffer, buffer solution and buffered solution, when used with reference to hydrogen-ion concentration or pH, refer to the ability of a system, particularly an aqueous solution, to resist a change of pH on adding acid or alkali, or on dilution with a solvent. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydroxyl ion added does not exceed the capacity of the buffer system to neutralize it.

Maintaining the pH of the formulation in the range of approximately 5.0 to 9.5 can enhance the stability of the parenteral formulation of the present invention. Other pH ranges, for example, include, 5.5 to 9.0, or 6.0 to 8.5, or 6.5 to 8.0, or 7.0 to 7.5.

The buffer used in the practice of the present invention is selected from any of the following, for example, an acetate buffer, a phosphate buffer or glutamate buffer, the most preferred buffer being a phosphate buffer.

Carriers or excipients can also be used to facilitate administration of the compositions and formulations of the invention. Examples of carriers and excipients include calcium carbonate, calcium phosphate, various sugars such as lactose, glucose, or sucrose, or types of starch, cellulose derivatives, gelatin, polyethylene glycols and physiologically compatible solvents.

A stabilizer may be included in the formulations of the invention, but will generally not be needed. If included, however, a stabilizer useful in the practice of the invention is a carbohydrate or a polyhydric alcohol. The polyhydric alcohols include such compounds as sorbitol, mannitol, glycerol, xylitol, and polypropylene/ethylene glycol copolymer, as well as various polyethylene glycols (PEG) of molecular weight 200, 400, 1450, 3350, 4000, 6000, and 8000). The carbohydrates include, for example, mannose, ribose, trehalose, maltose, inositol, lactose, galactose, arabinose, or lactose.

The United States Pharmacopeia (USP) states that anti-microbial agents in bacteriostatic or fungistatic concentrations must be added to preparations contained in multiple dose containers. They must be present in adequate concentration at the time of use to prevent the multiplication of microorganisms inadvertently introduced into the preparation while withdrawing a portion of the contents with a hypodermic needle and syringe, or using other invasive means for delivery, such as pen injectors. Antimicrobial agents should be evaluated to ensure compatibility with all other components of the formula, and their activity should be evaluated in the total formula to ensure that a particular agent that is effective in one formulation is not ineffective in another. It is not uncommon to find that a particular agent will be effective in one formulation but not effective in another formulation.

A preservative is, in the common pharmaceutical sense, a substance that prevents or inhibits microbial growth and may be added to a pharmaceutical formulation for this purpose to avoid consequent spoilage of the formulation by microorganisms. While the amount of the preservative is not great, it may nevertheless affect the overall stability of the copper antagonist.

While the preservative for use in the practice of the invention can range from 0.005 to 1.0% (w/v), the preferred range for each preservative, alone or in combination with others, is:benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid.

A detailed description of each preservative is set forth in “Remington's Pharmaceutical Sciences” as well as Pharmaceutical Dosage Forms:Parenteral Medications, Vol. 1, 1992, Avis et al. For these purposes, the copper antagonist may be administered parenterally (including subcutaneous injections, intravenous, intramuscular, intradermal injection or infusion techniques) or by inhalation spray in dosage unit formulations containing conventional non-toxic pharmaceutically-acceptable carriers, adjuvants and vehicles.

If desired, the parenteral formulation may be thickened with a thickening agent such as a methylcellulose. The formulation may be prepared in an emulsified form, either water in oil or oil in water. Any of a wide variety of pharmaceutically acceptable emulsifying agents may be employed including, for example, acacia powder, a non-ionic surfactant or an ionic surfactant.

It may also be desirable to add suitable dispersing or suspending agents to the pharmaceutical formulation. These may include, for example, aqueous suspensions such as synthetic and natural gums, e.g., tragacanth, acacia, alginate, dextran, sodium carboxymethylcellulose, methylcellulose, polyvinyl-pyrrolidone or gelatin.

A vehicle of importance for parenteral products is water. Water of suitable quality for parenteral administration must be prepared either by distillation or by reverse osmosis. Only by these means is it possible to separate adequately various liquid, gas and solid contaminating substances from water. The water may be purged with nitrogen gas to remove any oxygen or free radicals of oxygen from the water.

It is possible that other ingredients may be present in the parenteral pharmaceutical formulation of the invention. Such additional ingredients may include wetting agents, oils (e.g., a vegetable oil such as sesame, peanut or olive), analgesic agents, emulsifiers, antioxidants, bulking agents, tonicity modifiers, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Containers are also an integral part of the formulation of an injection and may be considered a component, for there is no container that is totally insoluble or does not in some way affect the liquid it contains, particularly if the liquid is aqueous. Therefore, the selection of a container for a particular injection must be based on a consideration of the composition of the container, as well as of the solution, and the treatment to which it will be subjected.

In order to permit introduction of a needle from a hypodermic syringe into a multiple-dose vial and provide for resealing as soon as the needle is withdrawn, each vial is sealed with a rubber closure held in place by an aluminum band.

Stoppers for glass vials, such as, West 4416/50, 4416/50 (Teflon faced) and 4406/40, Abbott 5139 or any equivalent stopper can be used as the closure for the dose vial. These stoppers pass the stopper integrity test when tested using patient use patterns, e.g., the stopper can withstand at least about 100 injections.

Each of the components of the pharmaceutical formulation described above is known in the art and is described in Pharmaceutical Dosage Forms:Parenteral Medications, Vol. 1, 2nd ed., Avis et al., Eds., Mercel Dekker, New York, N.Y. 1992.

The manufacturing process for the above formulation involves compounding, sterile filtration and filling steps. The compounding procedure, may for example, involve the dissolution of ingredients in a specific order, such as the preservative first followed by the stabilizer/tonicity agents, buffers and then the copper antagonist, or dissolving all of the ingredients forming the parenteral formulation at the same time. An example of one method of preparing a parenteral formulation for administration is the dissolution of the copper antagonist form, for example, a copper chelator(s), in water and diluting the resultant mixture to 154 mM in a phosphate buffered saline.

Alternatively, parenteral formulations of the invention are prepared by mixing the ingredients following generally accepted procedures. For example, the selected components may be mixed in a blender or other standard device to produce a concentrated mixture which may then be adjusted to the final concentration and viscosity by the addition of water, a thickening agent, a buffer, 5% human serum albumin or an additional solute to control tonicity.

Alternatively, the copper antagonist can be packaged as a dry solid and/or powder to be reconstituted with a solvent to yield a parenteral formulation in accordance with the invention for use at the time of reconstitution.

In addition the manufacturing process may include any suitable sterilization process when developing the parenteral formulation of the invention. Typical sterilization processes include filtration, steam (moist heat), dry heat, gases (e.g., ethylene oxide, formaldehyde, chlorine dioxide, propylene oxide, beta-propiolacctone, ozone, chloropicrin, peracetic acid methyl bromide and the like), radiant exposure and aseptic handling.

Suitable routes of parenteral administration include intramuscular, intravenous, subcutaneous, intraperitoneal, subdermal, intradermal, intraarticular, intrathecal and the like. Mucosal delivery is also permissible. The dose and dosage regimen will depend upon the weight and health of the subject.

In addition to the above means of achieving extended drug action, the rate and duration of copper chelator delivery may be controlled by, for example by using mechanically controlled drug infusion pumps.

The copper antagonist(s), such as, for example, a copper chelator(s), can be administered in the form of a depot injection that may be formulated in such a manner as to permit a sustained release of the copper antagonist. The copper antagonist can be compressed into pellets or small cylinders and implanted subcutaneously or intramuscularly. The pellets or cylinders may additionally be coated with a suitable biodegradable polymer chosen so as to provide a desired release profile. The copper antagonist may alternatively be micropelleted. The copper antagonist micropellets using bioacceptable polymers can be designed to allow release rates to be manipulated to provide a desired release profile. Alternatively, injectable depot forms can be made by forming microencapsulated matrices of the copper antagonist in biodegradable polymers such as polylactide-polyglycolide. Depending on the ratio of copper antagonist to polymer, and the nature of the particular polymer employed, the rate of copper antagonist release can be controlled. Examples of other biodegradable polymers include poly(orthoesters) and poly(anhydrides). Depot injectable formulations can also be prepared by entrapping the copper chelator in liposomes, examples of which include unilamellar vesicles, large unilamellar vesicles and multilamellar vesicles. Liposomes can be formed from a variety of phospholipids, such as cholesterol, stearyl amine or phosphatidylcholines. Depot injectable formulations can also be prepared by entrapping the copper chelator in microemulsions that are compatible with body tissue. By way of example reference is made to U.S. Pat. Nos. 6,410,041 and 6,362,190.

The invention in part provides infusion dose delivery formulations and devices, including but not limited to implantable infusion devices for delivery of compositions and formulations of the invention. Implantable infusion devices may employ inert material such as biodegradable polymers listed above or synthetic silicones, for example, cylastic, silicone rubber or other polymers manufactured by the Dow-Corning Corporation. The polymer may be loaded with copper antagonist and any excipients. Implantable infusion devices may also comprise a coating of, or a portion of, a medical device wherein the coating comprises the polymer loaded with trientine active agent and any excipient. Such an implantable infusion device may be prepared as disclosed in U.S. Pat. No. 6,309,380 by coating the device with an in vivo biocompatible and biodegradable or bioabsorbable or bioerodable liquid or gel solution containing a polymer with the solution comprising a desired dosage amount of copper antagonist and any excipients. The solution is converted to a film adhering to the medical device thereby forming the implantable copper antagonist-deliverable medical device.

An implantable infusion device may also be prepared by the in situ formation of a copper antagonist containing solid matrix as disclosed in U.S. Pat. No. 6,120,789, herein incorporated in its entirety. Implantable infusion devices may be passive or active. An active implantable infusion device may comprise a copper antagonist reservoir, a means of allowing the trientine active agent to exit the reservoir, for example a permeable membrane, and a driving force to propel the copper chelator from the reservoir. Such an active implantable infusion device may additionally be activated by an extrinsic signal, such as that disclosed in WO 02/45779, wherein the implantable infusion device comprises a system configured to deliver the copper antagonist comprising an external activation unit operable by a user to request activation of the implantable infusion device, including a controller to reject such a request prior to the expiration of a lockout interval. Examples of an active implantable infusion device include implantable drug pumps. Implantable drug pumps include, for example, miniature, computerized, programmable, refillable drug delivery systems with an attached catheter that inserts into a target organ system, usually the spinal cord or a vessel. See Medtronic Inc. Publications:UC9603124EN NP-2687, 1997; UC199503941b EN NP-2347 182577-101,2000; UC199801017a EN NP3273a 182600-101, 2000; UC200002512 EN NP4050, 2000; UC199900546bEN NP-3678EN, 2000. Minneapolis, Minn.:Medtronic Inc; 1997-2000. Many pumps have 2 ports:one into which drugs can be injected and the other that is connected directly to the catheter for bolus administration or analysis of fluid from the catheter. Implantable drug infusion pumps (SynchroMed EL and Synchromed programmable pumps; Medtronic) are indicated for long-term intrathecal infusion of morphine sulfate for the treatment of chronic intractable pain; intravascular infusion of floxuridine for treatment of primary or metastatic cancer; intrathecal injection (baclofen injection) for severe spasticity; long-term epidural infusion of morphine sulfate for treatment of chronic intractable pain; long-term intravascular infusion of doxorubicin, cisplatin, or methotrexate for the treatment or metastatic cancer; and long-term intravenous infusion of clindamycin for the treatment of osteomyelitis. Such pumps may also be used for the long-term infusion of one or more copper antagonists, for example, one or more copper chelators, at a desired amount for a desired number of doses or steady state administration. One form of a typical implantable drug infusion pump (Synchromed EL programmable pump; Medtronic) is titanium covered and roughly disk shaped, measures 85.2 mm in diameter and 22.86 mm in thickness, weighs 185 g, has a drug reservoir of 10 mL, and runs on a lithium thionyl-chloride battery with a 6- to 7-year life, depending on use. The downloadable memory contains programmed drug delivery parameters and calculated amount of drug remaining, which can be compared with actual amount of drug remaining to access accuracy of pump function, but actual pump function over time is not recorded. The pump is usually implanted in the right or left abdominal wall. Other pumps useful in the invention include, for example, portable disposable infuser pumps (PDIPs). Additionally, implantable infusion devices may employ liposome delivery systems, such as a small unilamellar vesicles, large unilamellar vesicles, and multilamellar vesicles can be formed from a variety of phospholipids, such as cholesterol, stearyl amine or phosphatidylcholines.

The invention also includes delayed-release ocular preparations containing one or more copper antagonist, for example, one or more copper chelators. One of the problems associated with the use of ophthalmic solutions is the rapid loss of administered drug due to blinking of the eye and the flushing effect of lacrimal fluids. Up to 80% of an administered dose may be lost through tears and the action of nasolacrimal drainage within 5 minutes of installation. Extended periods of therapy may be achieved by formulations of the invention that increase the contact time between the copper chelator and the corneal surface. This may be accomplished through use of agents that increase the viscosity of solutions; by ophthalmic suspensions in which the copper antagonist particles slowly dissolve; by slowly dissipating ophthalmic ointments; or by use of ophthalmic inserts. Preparations of one or more copper antagonist, for example, one or more copper chelators, suitable for ocular administration to humans may be formulated using synthetic high molecular weight cross-linked polymers such as those of acrylic acid (e.g., Carbopol 940) or gellan gum (Gelrite; see, Merck Index 12th Ed., 4389), a compound that forms a gel upon contact with the precorneal tear film (e.g. as employed in Timoptic-XE by Merck, Inc.).

Further examples include delayed-release ocular preparations containing copper antagonist in ophthalmic inserts, such as the OCUSERT system (Alza Inc.). Typically, such inserts are elliptical with dimensions of about 13.4 mm by 5.4 mm by 0.3 mm (thickness). The insert is flexible and has a copper antagonist-containing core surrounded on each side by a layer of hydrophobic ethylene/vinyl acetate copolymer membranes through which the copper antagonist diffuses at a constant rate. The white margin around such devices contains white titanium dioxide, an inert compound that confers visibility. The rate of copper antagonist diffusion is controlled by the polymer composition, the membrane thickness, and the copper antagonist solubility. During the first few hours after insertion, the copper antagonist release rate is greater than that which occurs thereafter in order to achieve initially therapeutic copper antagonist levels. The copper antagonist-containing inserts may be placed in the conjunctival sac from which they release their medication over a treatment period. Another form of an ophthalmic insert is a rod shaped, water-soluble structure composed of hydroxypropyl cellulose in which copper chelator is embedded. The insert is placed into the inferior cul-de-sac of the eye once or twice daily as required for therapeutic efficacy. The inserts soften and slowly dissolve, releasing the copper antagonist that is then taken up by the ocular fluids. A further example of such a device is furnished by Lacrisert (Merck Inc.).

The invention also provides in part dose delivery formulations and devices formulated to enhance bioavailability of copper antagonist. This may be in addition to or in combination with any of the formulations or devices described above.

Despite good hydrosolubility, one or more copper antagonists, such as a copper cheltaor, for example, trientine, may be poorly absorbed in the digestive tract. A therapeutically effective amount of copper antagonist is an amount capable of providing an appropriate level of copper antagonist in the bloodstream. By increasing the bioavailability of copper antagonist, a therapeutically effective level of copper antagonist may be achieved by administering lower dosages than would otherwise be necessary.

An increase in bioavailability of copper antagonist may be achieved by complexation of copper antagonist with one or more bioavailability or absorption enhancing agents or in bioavailability or absorption enhancing formulations.

The invention in part provides for the formulation of copper antagonist, e.g., copper chelator, with other agents useful to enhance bioavailability or absorption. Such bioavailability or absorption enhancing agents include, but are not limited to, various surfactants such as various triglycerides, such as from butter oil, monoglycerides, such as of stearic acid and vegetable oils, esters thereof, esters of fatty acids, propylene glycol esters, the polysorbates, sodium lauryl sulfate, sorbitan esters, sodium sulfosuccinate, among other compounds. By altering the surfactant properties of the delivery vehicle it is possible to, for example, allow a copper chelator to have greater intestinal contact over a longer period of time that increases uptake and reduces side effects. Further examples of such agents include carrier molecules such as cyclodextrin and derivatives thereof, well known in the art for their potential as complexation agents capable of altering the physicochemical attributes of drug molecules. For example, cyclodextrins may stabilize (both thermally and oxidatively), reduce the volatility of, and alter the solubility of, trientine active agents with which they are complexed. Cyclodextrins are cyclic molecules composed of glucopyranose ring units that form toroidal structures. The interior of the cyclodextrin molecule is hydrophobic and the exterior is hydrophilic, making the cyclodextrin molecule water-soluble. The degree of solubility can be altered through substitution of the hydroxyl groups on the exterior of the cyclodextrin. Similarly, the hydrophobicity of the interior can be altered through substitution, though generally the hydrophobic nature of the interior allows accommodation of relatively hydrophobic guests within the cavity. Accommodation of one molecule within another is known as complexation and the resulting product is referred to as an inclusion complex. Examples of cyclodextrin derivatives include sulfobutylcyclodextrin, maltosylcyclodextrin, hydroxypropylcyclodextrin, and salts thereof. Complexation of copper chelator with a carrier molecule such as cyclodextrin to form an inclusion complex may thereby reduce the size of the copper chelator dose needed for therapeutic efficacy by enhancing the bioavailability of the administered active agent.

The invention in part also provides for the formulation of copper antagonist, e.g., copper chelator, in a microemulsion to enhance bioavailability. A microemulsion is a fluid and stable homogeneous solution composed of four major constituents, respectively, a hydrophilic phase, a lipophilic phase, at least one surfactant (SA) and at least one cosurfactant (CoSA). A surfactant is a chemical compound possessing two groups, the first polar or ionic, which has a great affinity for water, the second which contains a longer or shorter aliphatic chain and is hydrophobic. These chemical compounds having marked hydrophilic character are intended to cause the formation of micelles in aqueous or oily solution. Examples of suitable surfactants include mono-, di- and triglycerides and polyethylene glycol (PEG) mono- and diesters. A cosurfactant, also sometimes known as “co-surface-active agent”, is a chemical compound having hydrophobic character, intended to cause the mutual solubilization of the aqueous and oily phases in a microemulsion. Examples of suitable co-surfactants include ethyl diglycol, lauric esters of propylene glycol, oleic esters of polyglycerol, and related compounds.

The invention in part also provides for the formulation of copper antagonist with various polymers to enhance bioavailability by increasing adhesion to mucosal surfaces, by decreasing the rate of degradation by hydrolysis or enzymatic degradation of the copper antagonist, and by increasing the surface area of the copper antagonist relative to the size of the particle. Suitable polymers can be natural or synthetic, and can be biodegradable or non-biodegradable. Delivery of low molecular weight active agents, such as for example compounds of Formulae I and II and trientine active agents, may occur by either diffusion or degredation of the polymeric system. Representative natural polymers include proteins such as zein, modified zein, casein, gelatin, gluten, serum albumin, and collagen, polysaccharides such as cellulose, dextrans, and polyhyaluronic acid. Synthetic polymers are generally preferred due to the better characterization of degradation and release profiles. Representative synthetic polymers include polyphosphazenes, poly(vinyl alcohols), polyamides, polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate). Synthetically modified natural polymers include cellulose derivatives such as alkyl celluloses, hydroxyalkyl celluloses, cellulose ethers, cellulose esters, and nitrocelluloses. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. Each of the polymers described above can be obtained from commercial sources such as Sigma Chemical Co., St. Louis, Mo., Polysciences, Warrenton, Pa., Aldrich Chemical Co., Milwaukee, Wis., Fluka, Ronkonkoma, N.Y., and BioRad, Richmond, Calif. or can be synthesized from monomers obtained from these suppliers using standard techniques. The polymers described above can be separately characterized as biodegradable, non-biodegradable, and bioadhesive polymers, as discussed in more detail below. Representative synthetic degradable polymers include polyhydroxy acids such as polylactides, polyglycolides and copolymers thereof, poly(ethylene terephthalate), poly(butic acid), poly(valeric acid), poly(lactide-co-caprolactone), polyanhydrides, polyorthoesters and blends and copolymers thereof. Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers. In general, these materials degrade either by enzymatic hydrolysis or exposure to water in vivo, by surface or bulk erosion. Examples of non-biodegradable polymers include ethylene vinyl acetate, poly(meth)acrylic acid, polyamides, polyethylene, polypropylene, polystyrene, polyvinyl chloride, polyvinylphenol, and copolymers and mixtures thereof. Hydrophilic polymers and hydrogels tend to have bioadhesive properties. Hydrophilic polymers that contain carboxylic groups (e.g., poly[acrylic acid]) tend to exhibit the best bioadhesive properties. Polymers with the highest concentrations of carboxylic groups are preferred when bioadhesiveness on soft tissues is desired. Various cellulose derivatives, such as sodium alginate, carboxymethylcellulose, hydroxymethylcellulose and methylcellulose also have bioadhesive properties. Some of these bioadhesive materials are water-soluble, while others are hydrogels. Polymers such as hydroxypropylmethylcellulose acetate succinate (HPMCAS), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP) may be utilized to enhance the bioavailibity of trientine active agent with which they are complexed. Rapidly bioerodible polymers such as poly(lactide-co-glycolide), polyanhydrides, and polyorthoesters, whose carboxylic groups are exposed on the external surface as their smooth surface erodes, can also be used for bioadhesive copper chelator delivery systems. In addition, polymers containing labile bonds, such as polyanhydrides and polyesters, are well known for their hydrolytic reactivity. Their hydrolytic degradation rates can generally be altered by simple changes in the polymer backbone. Upon degradation, these materials also expose carboxylic groups on their external surface, and accordingly, these can also be used for bioadhesive copper chelator delivery systems.

Other agents that may enhance bioavailability or absorption of one or more copper antagonists can act by facilitating or inhibiting transport across the intestinal mucosa. For example, it has long been suggested that blood flow in the stomach and intestine is a factor in determining intestinal drug absorption and drug bioavailability, so that agents that increase blood flow, such as vasodilators, may increase the rate of absorption of orally administered copper chelator by increasing the blood flow to the gastrointestinal tract. Vasodilators have been used in combination with other drugs. For example, in EPO Publication 106335, the use of a coronary vasodilator, diltiazem, is reported to increase oral bioavailability of drugs which have an absolute bioavailability of not more than 20%, such as adrenergic beta-blocking agents (e.g., propranolol), catecholamines (e.g., dopamine), benzodiazepine derivatives (e.g., diazepam), vasodilators (e.g., isosorbide dinitrate, nitroglycerin or amyl nitrite), cardiotonics or antidiabetic agents, bronchodilators (e.g., tetrahydroisoquinoline), hemostatics (e.g., carbazochrome sulfonic acid), antispasmodics (e.g., timepidium halide) and antitussives (e.g., tipepidine). Vasodilators therefore constitute another class of agents that may enhance the bioavailability of copper antagonist.

Other mechanisms of enhancing bioavailability of the compositions and formulations of the invention include the inhibition of reverse active transport mechanisms. For example, it is now thought that one of the active transport mechanisms present in the intestinal epithelial cells is p-glycoprotein transport mechanism which facilitates the reverse transport of substances, which have diffused or have been transported inside the epithelial cell, back into the lumen of the intestine. It has been speculated that the p-glycoprotein present in the intestinal epithelial cells may function as a protective reverse pump which prevents toxic substances which have been ingested and diffused or transported into the epithelial cell from being absorbed into the circulatory system and becoming bioavailable. One of the unfortunate aspects of the function of the p-glycoprotein in the intestinal cell however is that it can also function to prevent bioavailability of substances which are beneficial, such as certain drugs which happen to be substrates for the p-glycoprotein reverse transport system. Inhibition of this p-glycoprotein mediated active transport system will cause less drug to be transported back into the lumen and will thus increase the net drug transport across the gut epithelium and will increase the amount of drug ultimately available in the blood. Various p-glycoprotein inhibitors are well known and appreciated in the art. These include, water soluble vitamin E; polyethylene glycol; poloxamers including Pluronic F-68; Polyethylene oxide; polyoxyethylene castor oil derivatives including Cremophor EL and Cremophor RH 40; Chrysin, (+)-Taxifolin; Naringenin; Diosmin; Quercetin; and the like. Inhibition of a reverse active transport system of which, for example, a copper antagonist is a substrate may thereby enhance the bioavailability of said copper antagonist.

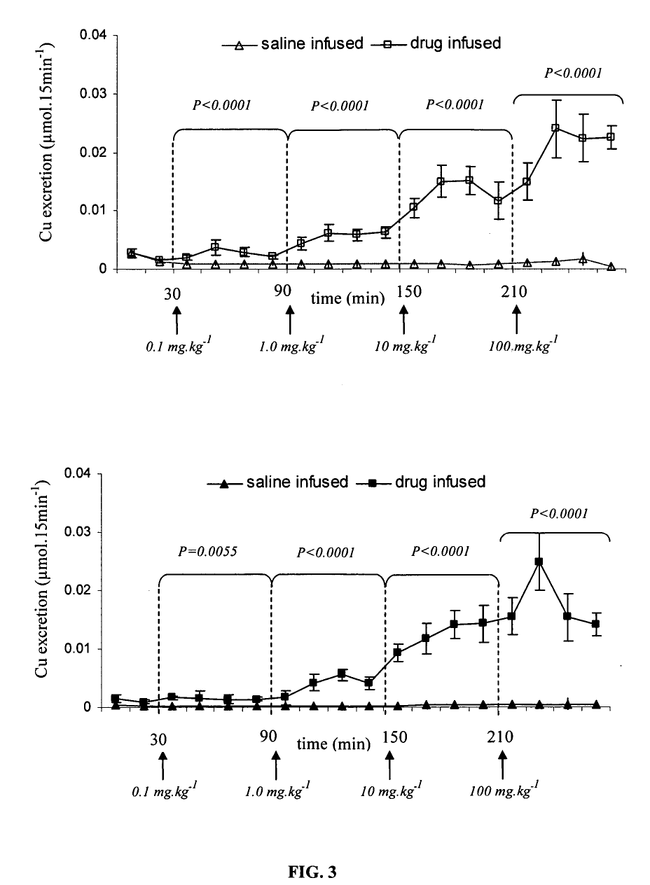

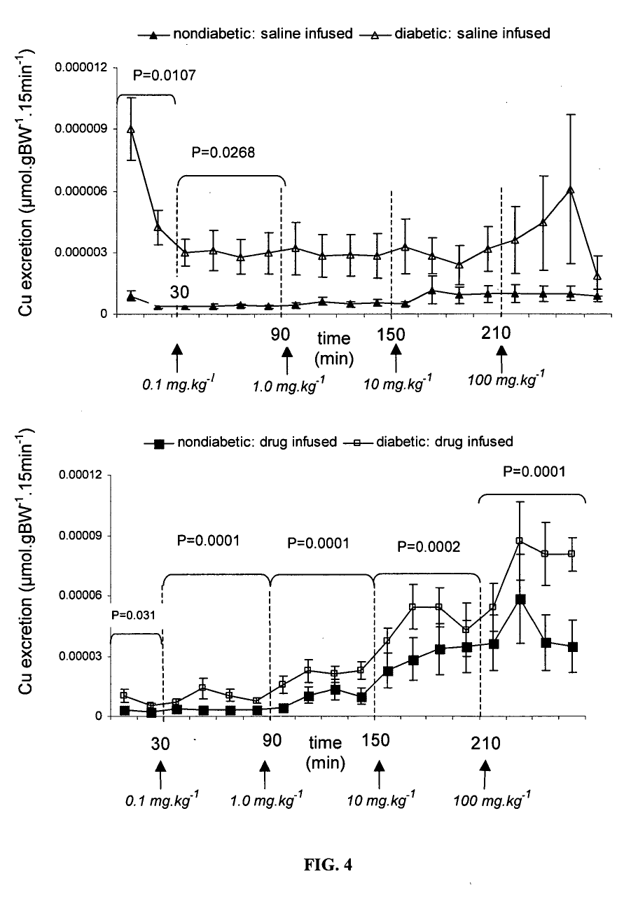

Surprisingly, as shown in Example 2, and in FIGS. 3 and 4 in particular, the copper chelator trientine dihydrochloride is effective at removing Cu from rats, including STZ-treated rats, at doses far lower than have been previously shown to be effective. As can be seen in FIG. 3 and particularly in FIG. 4, which presents Cu excretion normalised to body weight, Cu excretion in the urine of rats parenterally administered trientine dihydrochlorinde at a dose of 0.1 mg.kg⁻¹ (the lowest dose administered in the studies presented herein) is significantly increased over that of rats administered saline.

These data show that copper antagonists, including but not limited to trientine active agents, including but not limited to trientine, trientine salts, compounds of Formulae I and II, and so on, will be effective at doses lower than, for example, the doses herein shown to be effective in increasing Cn excretion in the urine of humans. It may be effective at doses in the order of 1/10, 1/100 and even 1/1000 of those we have already employed (e.g. in the order of 120 mg.d⁻¹, 12 mg.d⁻¹ or even 1.2 mg.d⁻¹).

The invention accordingly in part provides low-dose compositions, formulations and devices comprising one or more copper antagonist, for example one or more copper chelators, including but not limited to trientine active agents, including but not limited to trientine, trientine salts, componds of Formulae I and II, and so on, in an amount sufficient to provide, for example, dosage rates from 0.01 mg.kg⁻¹ to 5 mg.kg⁻¹, 0.01 mg.kg⁻¹ to 4.5 mg.kg⁻¹, 0.02 mg.kg⁻¹ to 4 mg.kg⁻¹, 0.02 to 3.5 mg.kg⁻¹, 0.02 mg.kg⁻¹ to 3 mg.kg⁻¹, 0.05 mg.kg⁻¹ to 2.5 mg.kg⁻¹, 0.05 mg.kg⁻¹ to 2 mg.kg⁻¹, 0.05-0.1 mg.kg⁻¹ to 5 mg.kg⁻¹, 0.05-0.1 mg.kg⁻¹ to 4 mg.kg⁻¹, 0.05-0.1 mg.kg⁻¹ to 3 mg.kg⁻¹, 0.05-0.1 mg.kg⁻¹ to 2 mg.kg⁻¹, 0.05-0.1 mg.kg⁻¹ to 1 mg.kg⁻¹, and/or any other rate within the ranges as set forth.

Any such dose may be administered by any of the routes or in any of the forms herein described. It will be appreciated that any of the dosage forms, compositions, formulations or devices described herein particularly for oral administration may be utilized, where applicable or desirable, in a dosage form, composition, formulation or device for administration by any of the other routes herein contemplated or commonly employed. For example, a dose or doses could be given parenterally using a dosage form suitable for parenteral administration which may incorporate features or compositions described in respect of dosage forms suitable for oral administration, or be delivered in an oral dosage form such as a modified release, extended release, delayed release, slow release or repeat action oral dosage form.

A better understanding of the invention will be gained by reference to the following experimental section. The following experiments are illustrative of the present invention and are not intended to limit the invention in any way.

### EXAMPLE 1

This Example was carried out to determine for the sake of subsequent comparison baseline physiological data relating to the effects of streptozotocin (STZ) treatment in rats.

All methods used in this study were approved by the University of Auckland Animal Ethics Committee and were in accordance with The Animals Protection Act and Regulations of New Zealand.

Male Wistar rats (n=28, 303±2.9 g) were divided randomly into STZ-treated and non-treated groups. Following induction of anesthesia (5% halothane and 2 l.min⁻¹ O₂), animals in the STZ-treated group received a single intravenous dose of streptozotocin (STZ, 55 mg.kg⁻¹ body weight, Sigma; St. Louis, Mo.) in 0.5 ml saline administered via the tail vein. Non-treated animals received an equivalent volume of saline. Following injection, both STZ-treated and non-treated rats were housed in like-pairs and provided with access to normal rat chow (Diet 86 pellets; New Zealand Stock Feeds, Auckland, NZ) and deionized water ad libitum. Blood glucose and body weight were measure at day 3 following STZ/saline injection and then weekly throughout the study.

Results were as follows. With regard to effects of STZ on blood glucose and body weight, blood glucose increased to 25±2 mmol.l⁻¹ three days following STZ injection (Table 1). Despite a greater daily food intake, STZ-treated animals lost weight whilst non-treated animals continued to gain weight during the 44 days following STZ/saline injection. On the day of the experiment blood glucose levels were 24±1 and 5±0 mmol.l⁻¹ and body weight 264±7 g and 434±9 g for STZ-treated and non-treated animals respectively.

TABLE 1
Blood glucose, body weight and food consumption
in STZ-treated versus non-treated animals

| | STZ-treated | | Non-treated | |
|---|---|---|---|---|
| Body weight prior to | 303 ± 3 | g | 303 ± 3 | g |
| STZ/saline | | | | |
| Blood glucose 3 days | *25 ± 2 | mmol · l⁻¹ | 5 ± 0.2 | mmol · l⁻¹ |
| following STZ/saline | | | | |
| Daily food consumption | *58 ± 1 | g | 28 ± 1 | g |
| Blood glucose on | *24 ± 1 | mmol · l⁻¹ | 5 ± 0.2 | mmol · l⁻¹ |
| experimental day | | | | |
| Body weight on | *264 ± 7 | g | 434 ± 9 | g |
| experimental day | | | | |

STZ-treated animals n = 14, non-treated animals n = 14.
Values shown as mean ± SEM.
Asterisk indicates a significant difference (P < 0.05).

Thus, results showed that STZ treatment resulted in elevated blood glucose, increased food intake, and decreased body weight.

### EXAMPLE 2

This Example assessed the effect of acute intravenous administration of increasing doses of trientine on the excretion profiles of copper and iron in the urine of STZ-treated and non-STZ-treated rats.

Six to seven weeks (mean=44±1 days) after administration of STZ, animals underwent either a control or trientine experimental protocol. All animals were fasted overnight prior to surgery but continued to have ad libitum access to deionized water. Induction and maintenance of surgical anesthesia was by 3-5% halothane and 2 l.min⁻¹ O₂. The femoral artery and vein were cannulated with a solid-state blood pressure transducer (Mikrotip™ 1.4F, Millar Instruments, Texas, USA) and a saline filled PE 50 catheter respectively. The ureters were exposed via a midline abdominal incision, cannulated using polyethylene catheters (external diameter 0.9 mm, internal diameter 0.5 mm) and the wound sutured closed. The trachea was cannulated and the animal ventilated at 70-80 breaths.min⁻¹ with air supplemented with O₂ (Pressure Controlled Ventilator, Kent Scientific, Conn., USA). The respiratory rate and end-tidal pressure (10-15 cmH₂O) were adjusted to maintain end-tidal CO₂ at 35-40 mmHg (SC-300 CO₂ Monitor, Pryon Corporation, Wisconsin, USA). Body temperature was maintained at 37° C. throughout surgery and the experiment by a heating pad. Estimated fluid loss was replaced with intravenous administration of 154 mmol.l⁻¹ NaCl solution at a rate of 5 ml.kg⁻¹.h⁻¹.

Following surgery and a 20 min stabilization period, the experimental protocol was started. Trientine was administered intravenously over 60 s in hourly doses of increasing concentration (0.1, 1.0, 10 and 100 mg.kg-1 in 75 μl saline followed by 125 μl saline flush). Control animals received an equivalent volume of saline. Urine was collected in 15 min aliquots throughout the experiment in pre-weighed polyethylene epindorf tubes. At the end of the experiment a terminal blood sample was taken by cardiac puncture and the separated serum stored at −80° C. until future analysis. Hearts were removed through a rapid mid-sternal thoracotomy and processed as described below.

Mean arterial pressure (MAP), heart rate (HR, derived from the MAP waveform) oxygen saturation (Nonin 8600V Pulse Oximeter, Nonin Medical Inc., Minnesota, USA) and core body temperature, were all continuously monitored throughout the experiment using a PowerLab/16s data acquisition module (AD Instruments, Australia). Calibrated signals were displayed on screen and saved to disc as 2 s averages of each variable.

Urine and tissue analysis was carried out as follows. Instrumentation:A Perkin Elmer (PE) Model 3100 Atomic Absorption Spectrophotometer equipped with a PE HGA-600 Graphite Furnace and PE AS-60 Furnace Autosampler was used for Cu and Fe determninations in urine. Deuterium background correction was employed. A Cu or Fe hollow-cathode lamp (Perkin Elmer Corporation) was used and operated at either 10 W (Cu) or 15 W (Fe). The 324.8 nm atomic line was used for Cu and the 248.3 nm atomic line for Fe. The slit width for both Cu and Fe was 0.7 nm. Pyrolytically coated graphite tubes were used for all analyses. The injection volume was 20 μL. A typical graphite furnace temperature program is shown below:

GF-AAS temperature program

| Procedure | Temp/° C. | Ramp/s | Hold/s | Int. Flow/mL min⁻¹ |
|---|---|---|---|---|
| Drying | 90 | 1 | 5 | 300 |
| | 120 | 60 | 5 | 300 |
| Pre-treatment | 1250* | 20 | 10 | 300 |
| | 20 | 1 | 10 | 300 |
| Atomization - Cu/Fe | 2300/2500 | 1 | 8 | 0 |
| Post-treatment | 2600 | 1 | 5 | 300 |

*A pre-treatment temperature of 1050° C. was used for tissue digest analyses (see Example 3)

Reagents:All reagents used were of the highest purity available and at least of analytical grade. GF-AAS standard working solutions of Cu and Fe were prepared by stepwise dilution of 1000 mg.l⁻¹ (Spectrosol standard solutions; BDH). Water was purified by a Millipore Milli-Q ultra-pure water system to a resistivity of 18 MΩ.

Sample pretreatment was carried out as follows. Urine:Urine was collected in pre-weighed 1.5 ml micro test tubes (eppendorf). After reweighing, the urine specimens were centrifuged and the supernatant diluted 25:1 with 0.02 M 69% Aristar grade HNO₃. The sample was stored at 4° C. prior to GF-AAS analysis. If it was necessary to store a sample for a period in excess of 2 weeks, it was frozen and kept at −20° C. Serum:Terminal blood samples were centrifuged and serum treated and stored as per urine until analysis. From the trace metal content of serum from the terminal blood sample and urine collected over the final hour of the experiment, renal clearance was calculated using the following equation:

$$renal clearance of trace metal ( µl . {min}^{- 1} ) = \frac{\begin{matrix}concentration of metal in urine ( µg . {µl}^{- 1} ) * \\ rate of urine flow ( µl . {min}^{- 1} )\end{matrix}}{concentration of metal in serum ( µg . {µl}^{- 1} )}$$

Statistical analyses were carried out as follows. All values are expressed as mean±SEM and P values <0.05 were considered statistically significant. Student's unpaired t-test was initially used to test for weight and glucose differences between the STZ-treated and control groups. For comparison of responses during drug exposure, statistical analyses were performed using analysis of variance (Statistics for Windows v.6.1, SAS Institute Inc., California, USA). Subsequent statistical analysis was performed using a mixed model repeated measures ANOVA design (see Example 4).

The results were as follows. With regard to cardiovascular variables during infusion, baseline levels of MAP during the control period prior to infusion were not significantly different between non-STZ-treated and STZ-treated animals (99±4 mmHg). HR was significantly lower in STZ-treated than non-STZ-treated animals (287±11 and 364±9 bpm respectively, P<0.001). Infusion of trientine or saline had no effect on these variables except at the highest dose where MAP decreased by a maximum of 19±4 mmHg for the 2 min following administration and returned to pre-dose levels within 10 min. Body temperature and oxygen saturation remained stable in all animals throughout the experiment.

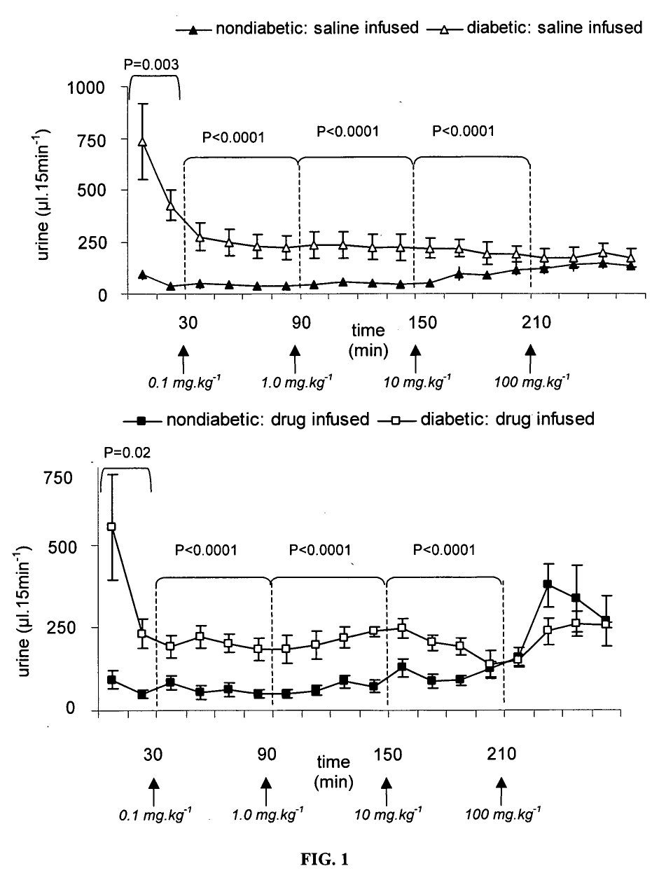

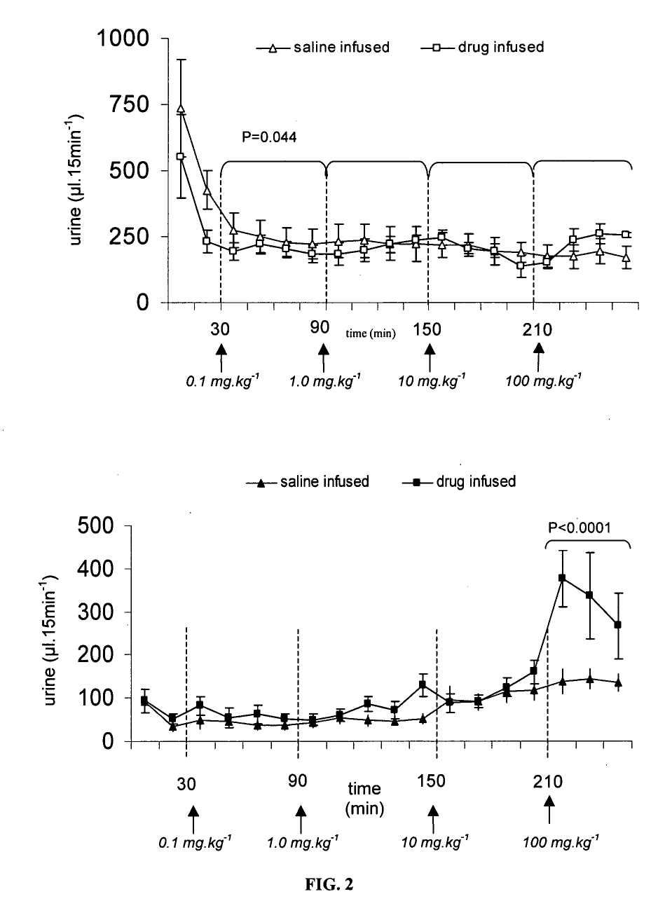

With regard to urine excretion, STZ-treated animals consistently excreted significantly more urine than non-STZ-treated animals except in response to the highest dose of copper chelator (100 mg.kg⁻¹) or equivalent volume of saline (FIG. 1). Administration of the 100 mg.kg⁻¹ dose of trientine also increased urine excretion in non-STZ-treated animals to greater than that of non-STZ-treated animals receiving the equivalent volume of saline (FIG. 2). This effect was not seen in STZ-treated animals.

With regard to urinary excretion of Cu and Fe analysis of the dose response curves showed that, at all doses, STZ-treated and non-STZ-treated animals receiving copper chelator excreted more Cu than animals receiving an equivalent volume of saline (FIG. 3). To provide some correction for the effects of lesser total body growth of the STZ-treated animals, and thus to allow more appropriate comparison between STZ-treated and non-STZ-treated animals, excretion rates of trace elements were also calculated per gram of body weight. FIG. 4 shows that STZ-treated animals had significantly greater copper excretion per gram of body weight in response to each dose of copper chelator than did non-STZ-treated animals. The same pattern was seen in response to saline, however the effect was not always significant.

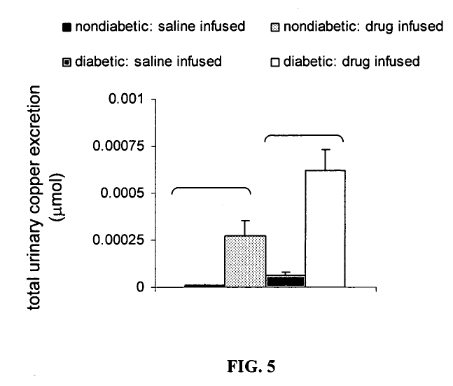

Total copper excreted over the entire duration of the experiment was significantly increased in both non-STZ-treated and STZ-treated animals administered trientine compared with their respective saline controls (FIG. 5). STZ-treated animals receiving copper chelator also excreted more total copper per gram of body weight than non-STZ-treated animals receiving copper chelator. The same significant trend was seen in response to saline administration (FIG. 6).

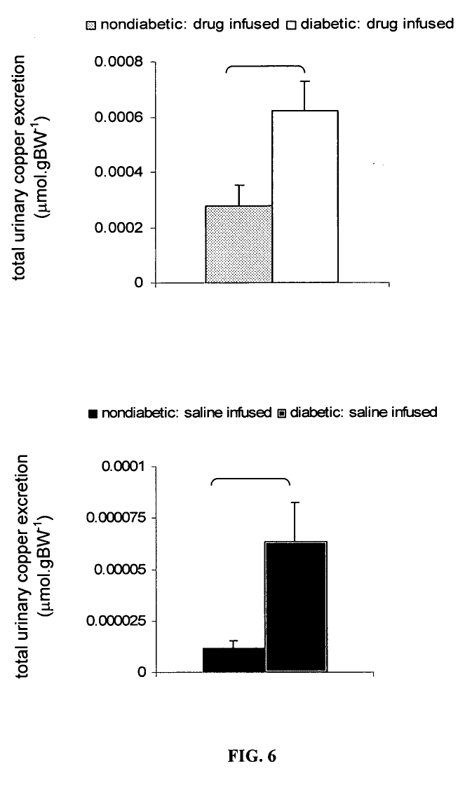

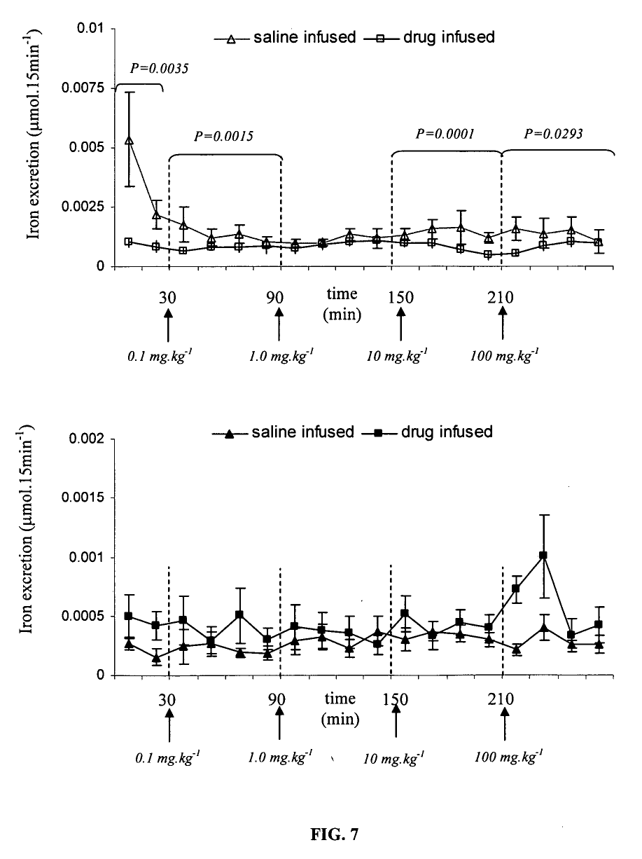

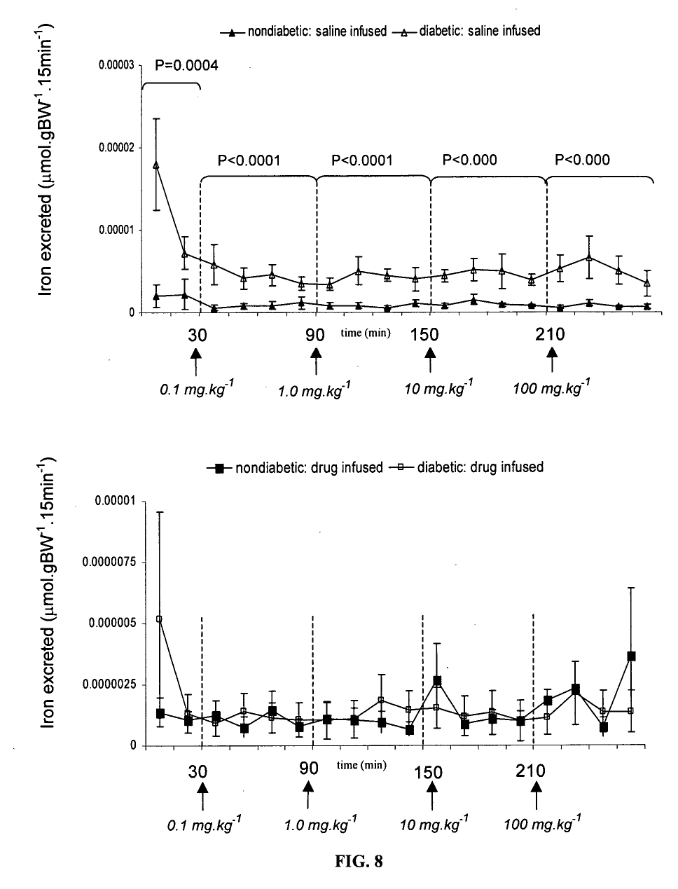

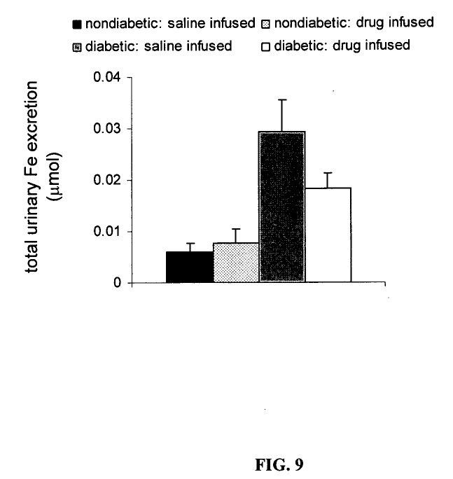

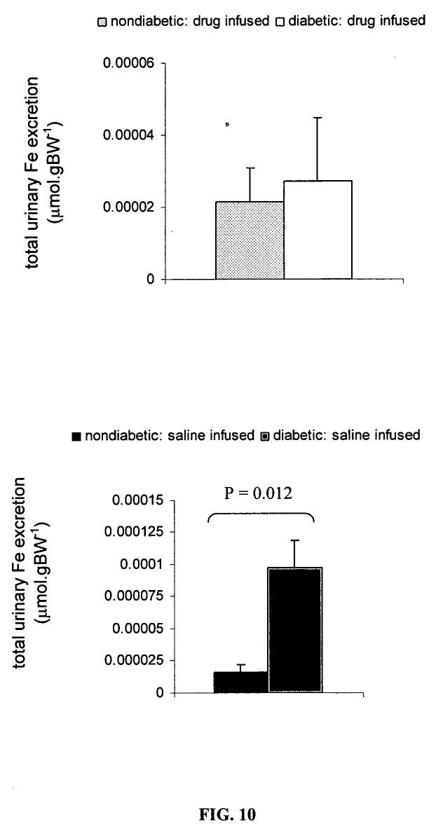

In comparison, iron excretion in both STZ-treated and non-STZ-treated animals receiving trientine was not greater than animals receiving an equivalent volume of saline (FIG. 7). Analysis per gram of body weight shows STZ-treated animals receiving saline excrete significantly more iron than non-STZ-treated animals, however this trend was not evident between STZ-treated and non-STZ-treated animals receiving trientine (FIG. 8). Total iron excretion in both STZ-treated and non-STZ-treated animals receiving copper chelator was not different from animals receiving saline (FIG. 9). In agreement with analysis of dose response curves, total iron excretion per gram of body weight was significantly greater in STZ-treated animals receiving saline than non-STZ-treated animals but this difference was not seen in response to trientine (FIG. 10).

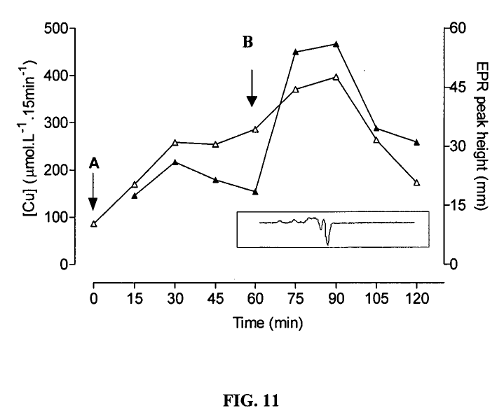

Electron paramagnetic resonance spectroscopy showed that the urinary Cu from copper chelator-treated animals was mainly complexed as trientine-Cu^{II} (FIG. 11), indicating that the increased tissue Cu in STZ-treated rats is mainly divalent. These data indicate that rats with severe hyperglycaemia develop increased systemic Cu^{II} that can be extracted by selective chelation.

With regard to Serum content and renal clearance of Cu and Fe, while there was no significant difference in serum copper content, there was a significant increase in renal clearance of copper in STZ-treated animals receiving copper chelator compared with STZ-treated animals receiving saline (Table 2). The same pattern was seen in non-STZ-treated animals, although the trend was not statistically significant (P=0.056). There was no effect of copper chelator or state (STZ-treated versus non-STZ-treated) on serum content or renal clearance of iron.

TABLE 2
Serum content and renal clearance of Cu and Fe in STZ-treated
and non-STZ-treated animals receiving drug or saline.

| | STZ- | STZ- | non-STZ- | non-STZ- |
|---|---|---|---|---|
| | treated | treated | treated | treated |
| | trientine | Saline | trientine | Saline |
| | n = 6 | n = 7 | n = 4 | n = 7 |
| Serum Cu | 7.56 ± 0.06 | 9.07 ± 1.74 | 7.11 ± 0.41 | 7.56 ± 0.62 |
| (μg · μl⁻¹ × | | | | |
| 10⁻⁴) | | | | |
| Serum Fe | 35.7 ± 7.98 | 63.2 ± 16.4 | 33.6 ± 1.62 | 31.4 ± 8.17 |
| (μg · μl⁻¹ × | | | | |
| 10⁻⁴) | | | | |
| Renal clear- | *28.5 ± 4.8 | 1.66 ± 0.82 | 19.9 ± 6.4 | 0.58 ± 0.28 |
| ance Cu | | | | |
| (μl · min⁻¹) | | | | |
| Renal clear- | 0.25 ± 0.07 | 0.38 ± 0.15 | 0.46 ± 0.22 | 0.11 ± 0.03 |
| ance Fe | | | | |
| (μl · min⁻¹) | | | | |

Values shown as mean±SEM. Asterisk indicates a significant difference (P<0.05) between STZ-treated animals receiving trientine and STZ-treated animals receiving an equivalent volume of saline.

In summary, acute intravenous administration of trientine significantly increased total copper excretion in both non-STZ-treated and STZ-treated animals compared with their respective saline controls. Furthermore, following acute intravenous administration of increasing doses of trientine, STZ-treated animals had significantly greater copper excretion per gram of body weight than did non-STZ-treated animals. In contrast, total iron excretion in both STZ-treated and non-STZ-treated animals receiving drug was not different from animals receiving saline.

### EXAMPLE 3

This example was carried out to determine the effect of acute intravenous administration of increasing doses of trientine on the copper and iron content of cardiac tissue in STZ-treated and non-STZ-treated rats, and to assess the effect of trientine on tissue repair.

Methods were carried out as follows. Spectrophotometric analysis was conducted as described in Example 2. Cu, Fe and Zn in tissue digests were determined at Hill Laboratories (Hamilton, New Zealand) using either a PE Sciex Elan-6000 or PE Sciex Elan-6100 DRC ICP-MS. The operating parameters are summarized in the Table below.

Instrumental operating parameters for ICP-MS

| Parameter | Value | |
|---|---|---|
| Inductively coupled plasma | | |
| Radiofrequency power | 1500 | W |
| Argon plasma gas flow rate | 15 | l · min⁻¹ |
| Argon auxiliary gas flow rate | 1.2 | l · min⁻¹ |
| Argon nebuliser gas flow rate | 0.89 | l · min⁻¹ |
| Interface | | |
| Sampler cone and orifice diameter | Ni/1.1 | mm |
| Skimmer cone and orifice diameter | Ni/0.9 | mm |
| Data acquisition parameters | | |
| Scanning mode | Peak hopping | |
| Dwell time | 30 | ms (Cu, Zn)/100 ms (Fe) |
| Sweeps/replicate | 20 | |
| Replicates | 3 | |
| Sample uptake rate | 1 | ml · min⁻¹ |

Reagents were as follows. Standard Reference Material 1577b Bovine Liver was obtained from the National Institute of Standards and Technology and used to evaluate the efficiency of tissue digestion. The results obtained are reported below:

GF-AAS and ICP-MS results for NIST SRM 1577b bovine liver*

| Element | Certified value | GF-AAS | ICP-MS |
|---|---|---|---|
| Cu | 160 ± 8 | 142 ± 12 | 164 ± 12 |
| Fe | 184 ± 15 | 182 ± 21 | 166 ± 14 |
| Zn | 127 ± 16 | — | 155 ± 42 |

*Measured in μg · g⁻¹ of dry matter.

Sample pre-treatment was carried out as follows. Heart:Following removal from the animal, the heart was cleaned of excess tissue, rinsed in buffer to remove excess blood, blotted dry and a wet ventricular weight recorded. Using titanium instruments a segment of left ventricular muscle was dissected and placed in a pre-weighed 5.0 ml polystyrene tube. The sample was freeze-dried overnight to constant weight before 0.45 ml of 69% Aristar grade HNO₃ was added. The sample tube was heated in a water bath at 65° C. for 60 minutes. The sample was brought to 4.5 ml with Milli-Q H₂O. The resulting solution was diluted 2:1 in order to reduce the HNO₃ concentration below the maximum permitted for ICP-MS analysis.

The results were as follows. With regard to the metal content of cardiac tissue, wet heart weights in STZ-treated animals were significantly less than those in non-STZ-treated animals while heart/body weight ratios were increased (see Table 3). Cardiac tissue from some animals was also analysed for Cu and Fe content. There was no significant difference in content of copper between STZ-treated and non-STZ-treated animals receiving saline or trientine. Iron content of the non-STZ-treated animals administered saline was significantly greater than that of the STZ-treated animals administered saline (see Table 3).

TABLE 3
Heart weight, heart weight/body weight ratios and trace metal content
of heart tissue in STZ-treated versus non-STZ-treated animals

| | STZ-treated | | Non STZ-treated | |
|---|---|---|---|---|
| Wet heart weight | *0.78 ± 0.02 | g | 1.00 ± 0.02 | g |
| Heart weight/body | *2.93 ± 0.05 | mg · g⁻¹ | 2.30 ± 0.03 | mg · g⁻¹ |
| weight | | | | |
| Cu content μg · g⁻¹ | | | | |
| dry tissue | | | | |
| Trientine treated | 24.7 ± 1.5 | | 27.1 ± 1.0 | |
| Saline treated | 21.3 ± 0.9 | | 27.2 ± 0.7 | |
| Fe content μg · g⁻¹ | | | | |
| dry tissue | | | | |
| Trientine treated | 186 ± 46 | | 235 ± 39 | |
| Saline treated | ^{†}180 ± 35 | | 274 ± 30 | |

STZ-treated animals: n = 14; non-STZ-treated animals: n = 14.
Values shown as mean ± SEM.
Asterisk indicates a significant difference (P < 0.05) between STZ-treated and non-STZ-treated animals.
^{†}indicates a significant difference (P < 0.05) between STZ-treated and non-STZ-treated animals receiving saline.

In summary, it was demonstrated that acute intravenous administration of increasing doses of trientine had no significant effect on the copper content of cardiac tissue in normal and STZ-treated rats.

### EXAMPLE 4

In this Example, a mixed linear model was applied to the data generated above in Examples 1-3.

Methods were as follows. With regard to statistical analysis using a mixed linear model, data for each dose level were analyzed using a mixed linear model (PROC MIXED; SAS, Version 8). The model included STZ-treatment, trientine and their interaction as fixed effects, time as a repeated measure, and rats as the subjects in the dataset. Complete independence was assumed across subjects. The full model was fitted to each dataset using a maximum likelihood estimation method (REML) fits mixed linear models (i.e., fixed and random effects models). A mixed model is a generalization of the standard linear model, the generalization being that one can analyze data generated from several sources of variation instead of just one. A level of significance of 0.05 was used for all tests. Results were as follows.

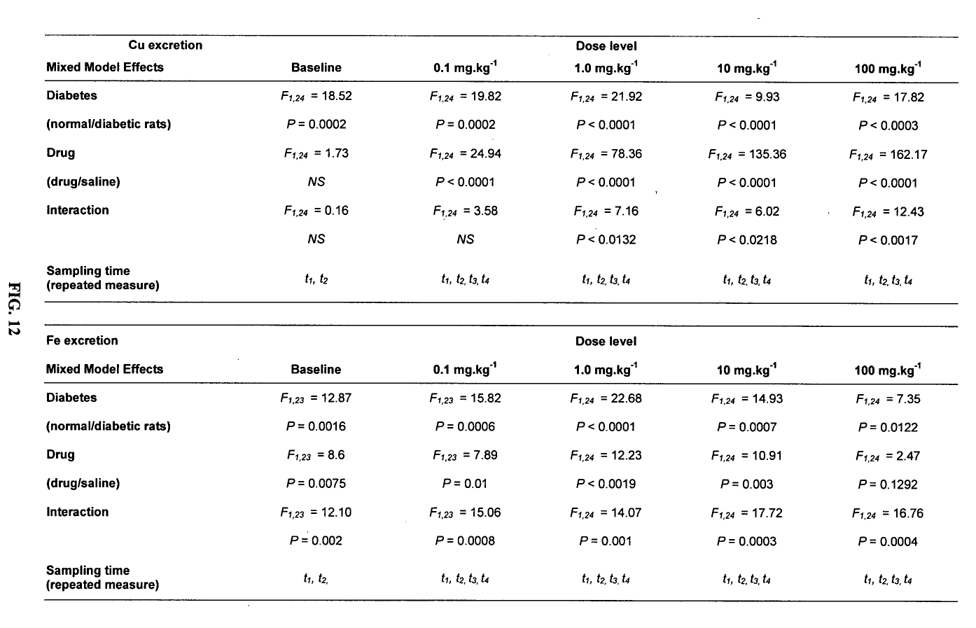

With regard to copper, STZ-treated rats excreted significantly higher levels of copper across all dose levels (see FIG. 12). Baseline copper excretion was also significantly higher in STZ-treated rats compared to non-STZ-treated rats. There was no difference at baseline levels between the trientine and saline groups. The interaction effect for the model was significant at dose levels of 1.0 mg.kg⁻¹ and above. The presence of a significant interaction term means that the influence of one effect varies with the level of the other effect. Therefore, the outcome of a significant interaction between the STZ-treatment and trientine factors is increased copper excretion above the predicted additive effects of these two factors.

With regard to iron, STZ-treated rats in the saline only group excreted significantly higher levels of iron at all dose levels. This resulted in all factors in the model being significant across all dose levels.

In sum, the acute effect of intravenous trientine administration on the cardiovascular system and urinary excretion of copper and iron was studied in anesthetized, STZ-treated and non-STZ-treated rats. Animals were assigned to one of four groups:STZ-treated+trientine, STZ-treated+saline, non-STZ-treated+trientine, non-STZ-treated+saline. Trientine, or an equivalent volume of saline, was administered hourly in doses of increasing strength (0.1, 1.0, 10, 100 mg.kg⁻¹) and urine was collected throughout the experiment in 15 min aliquots. A terminal blood sample was taken and cardiac tissue harvested. Analysis of urine samples revealed:(1) At all trientine doses, STZ-treated and non-STZ-treated animals receiving trientine excreted more Cu (μmol) than animals receiving an equivalent volume of saline; (2) When analyzed per gram of bodyweight, STZ-treated animals excreted significantly more copper (μmol.gBW⁻¹) at each dose of trientine than did non-STZ-treated animals. The same pattern was seen in response to saline but the effect was not significant at every dose; (3) At most doses, in STZ-treated animals iron excretion (μmol) was greater in animals administered saline than in those administered trientine. In non-STZ-treated animals there was no difference between iron excretion in response to saline or trientine administration; (4) Analysis per gram of body weight shows no difference between iron excretion in non-STZ-treated and STZ-treated animals receiving trientine. STZ-treated animals receiving saline excrete more iron per gram of bodyweight than non-STZ-treated animals receiving saline; (5) Analysis of heart tissue showed no significant difference in total copper content between STZ-treated and non-STZ-treated animals, nor any effect of trientine on cardiac content of iron and copper; and (6) Renal clearance calculations showed a significant increase in clearance of copper in STZ-treated animals receiving trientine compared with STZ-treated animals receiving saline. The same trend was seen in non-STZ-treated animals but the affect was not significant. There was no effect of trientine on renal clearance of iron.

There were no adverse cardiovascular effects observed after acute administration of trientine. Trientine treatment effectively increases copper excretion in both STZ-treated and non-STZ-treated animals. The excretion of copper in urine following trientine administration is greater per gram of bodyweight in STZ-treated than in non-STZ-treated animals. Iron excretion was not increased by trientine treatment in either STZ-treated or non-STZ-treated animals.

### EXAMPLE 5

Experiments relating to the efficacy of trientine to enhance tissue repair and/or restore organ function, for example, cardiac function, in STZ-treated rats were carried out. As noted therein, histological evidence showed that treatment with trientine appears to protect the hearts of STZ-treated Wistar rats from development of cardiac damage (diabetic cardiomyopathy) and/or enhance tissue repair in the hearts of said rats, as judged by histology. However, it was unknown whether this histological improvement may lead to improved cardiac function.

This experiment was carried out to compare cardiac function in trientine-treated and non-treated, STZ-treated and normal rats using an isolated-working-rodent heart model.

Methods were as follows. The animals used in these experiments received care that complied with the “Principles of Laboratory Animal Care” (National Society for Medical Research), and the University of Auckland Animal Ethics Committee approved the study.

Male albino Wistar rats weighing 330-430 g were assigned to four experimental groups as shown in Table 4.

TABLE 4
Experimental groups

| Group | Code | N | Treatment |
|---|---|---|---|
| Group A | STZ | 8 | STZ-induced diabetes for |
| | | | 13 weeks |
| Group B | STZ/D7 | 8 | STZ-induced diabetes for |
| | | | 13 weeks (Trientine therapy |
| | | | week 7-13) |
| Group C | Sham | 9 | Non-STZ-treated controls |
| Group D | Sham/D7 | 11 | Non-STZ-treated controls |
| | | | (Trientine therapy week 7- |
| | | | 13) |

STZ = Streptozotocin;
D7 = trientine treatment for 7 consecutive weeks commencing 6 weeks after the start of the experiment.

Diabetes was induced by intravenous streptozotocin (STZ; Sigma; St. Louis, Mo.). All rats were given a short inhalational anesthetic (Induction:5% halothane and 2 L/min oxygen, maintained on 2% halothane and 2 L/min oxygen). Those in the two STZ-treated groups then received a single intravenous bolus dose of STZ (57 mg/kg body weight) in 0.5 ml of 0.9% saline administered via a tail vein. Non-STZ-treated sham-treated animals received an equivalent volume of 0.9% saline. STZ-treated and non-STZ-treated rats were housed in like-pairs and provided with free access to normal rat chow (Diet 86 pellets; New Zealand Stock Feeds, Auckland, NZ) and deionized water ad libitum. Each cage had two water bottles on it to ensure equal access to water or trientine for each animal. Animals were housed at 21 degrees 37° C. and 60% humidity in standard rat cages with a sawdust floor that was changed daily.

Blood glucose was measured in tail-tip capillary blood samples (Advantage II, Roche Diagnostics, NZ Ltd). Sampling was performed on all groups at the same time of the day. Blood glucose and body weight were measured on day 3 following STZ/saline injection and then weekly throughout the study. Diabetes was confirmed by presence of polydipsia, polyuria and hyperglycemia (>11 mmol.L⁻¹).

In the trientine treated STZ-treated group, trientine was prepared in the drinking water for each cage at a concentration of 50 mg/L. The trientine-containing drinking water was administered continuously from the start of week 7 until the animal was sacrificed at the end of week 13. In the case of the Sham/D7 non-STZ-treated group that drank less water per day than STZ-treated animals, the trientine concentration in their drinking water was adjusted so that they consumed approximately the same dose as the corresponding STZ/D7 group. Trientine treated animals ingested mean trientine doses of between 8 to 11 mg per day.

At the time the trientine started in the STZ-treated group the STZ-treated animals were expected to have to have established cardiomyopathy, as shown by preliminary studies (data not shown) and confirmed in the literature. See Rodrigues B, et al., Diabetes 37(10):1358-64 (1988).

On the last day of the experiment, animals were anesthetized (5% halothane and 2 L.min⁻¹ O₂), and heparin (500 IU.kg⁻¹) (Weddel Pharmaceutical Ltd., London) administered intravenously via tail vein. A 2 ml blood sample was then taken from the inferior vena cava and the heart was then rapidly excised and immersed in ice-cold Krebs-Henseleit bicarbonate buffer to arrest contractile activity. Hearts were then placed in the isolated perfused working heart apparatus.

The aortic root of the heart was immediately ligated to the aortic cannula of the perfusion apparatus. Retrograde (Langendorff) perfusion at a hydrostatic pressure of 100 cm H₂O and at 37° C. was established and continued for 5 min while cannulation of the left atrium via the pulmonary vein was completed. The non-working (Langendorff) preparation was then converted to the working heart model by switching the supply of perfusate buffer from the aorta to the left atrium at a filling pressure of 10 cm H₂O. The left ventricle spontaneously ejected into the aortic cannula against a hydrostatic pressure (after-load) of 76 cmH₂O (55.9 mmHg). The perfusion solution was Krebs-Henseleit bicarbonate buffer (mM:KCl 4.7, CaCl₂ 2.3, KH₂PO₄ 1.2, MgSO₄ 1.2, NaCl 118, and NaHCO₃ 25), pH 7.4 containing 11 mM glucose and it was continuously gassed with 95% O₂:5% CO₂. The buffer was also continuously filtered in-line (initial 8 μm, following 0.4 μm cellulose acetate filters; Sartorius, Germany). The temperature of the entire perfusion apparatus was maintained by water jackets and buffer temperature was continuously monitored and adjusted to maintain hearts at 37° C. throughout perfusion.

A modified 24 g plastic intravenous cannula (Becton Dickson, Utah, USA) was inserted into the left ventricle via the apex of the heart using the normal introducer-needle. This cannula was subsequently attached to a SP844 piezo-electric pressure transducer (AD Instruments) to continuously monitor left ventricular pressure. Aortic pressure was continuously monitored through a side arm of the aortic cannula with a pressure transducer (Statham Model P23XL, Gould Inc., CA, USA). The heart was paced (Digitimer Ltd, Heredfordshire, England) at a rate of 300 bpm by means of electrodes attached to the aortic and pulmonary vein cannulae using supra-threshold voltages with pulses of 5-ms duration from the square wave generator.

Aortic flow was recorded by an in-line flow meter (Transonic T206, Ithaca, N.Y., USA) and coronary flow was measured by timed 30 sec collection of the coronary vein effluent at each time point step of the protocol.

The working heart apparatus used was a variant of that originally described by Neely, J R, et al., Am J Physiol 212:804-14 (1967). The modified apparatus allowed measurements of cardiac function at different pre-load pressures. This was achieved by constructing the apparatus so that the inflow height of the buffer coming to the heart could be altered through a series of graduated steps in a reproducible manner. As in the case of the pre-load, the outflow tubing from the aorta could also be increased in height to provide a series of defined after-load pressures. The after-load heights have been converted to mm Hg for presentation in the results which is in keeping with published convention.

All data from the pressure transducers and flow probe were collected (Powerlab 16s data acquisition machine; AD Instruments, Australia). The data processing functions of this device were used to calculate the first derivative of the two pressure waves (ventricular and aortic). The final cardiac function data available comprised:

Cardiac output*; aortic flow; coronary flow; peak left ventricular/aortic pressure developed; maximum rate of ventricular pressure development (+dP/dt)**; maximum rate of ventricular pressure relaxation (−dP/dt)**; maximum rate of aortic pressure development (aortic +dP/dt); maximum rate of aortic relaxation (aortic −dP/dt). [*Cardiac output (CO) is the amount of buffer pumped per unit time by the heart and is comprised of buffer that is pumped out the aorta as well as the buffer pumped into the coronary vessels. This is an overall indicator of cardiac function. **+dP/dt is the rate of change of ventricular (or aortic pressure) and correlates well with the strength of the contraction of the ventricle (contractility). It can be used to compare contractility abilities of different hearts when at the same pre-load (Textbook of Medical Physiology, Ed. A. Guyton. Saunders company 1986). −dP/dt is an accepted measurement of the rate of relaxation of the ventricle].

The experiment was divided into two parts, the first with fixed after-load and variable pre-load the second, which immediately followed on from the first, with fixed pre-load and variable after-load.

Fixed After-load and changing Pre-load:After the initial cannulation was completed, the heart was initially allowed to equilibrate for 6 min at 10 cm H₂O atrial filling pressure and 76 cm H₂O after-load. During this period the left ventricular pressure transducer cannula was inserted and the pacing unit started. Once the heart was stable, the atrial filling pressure was then reduced to 5 cm H₂O of water and then progressively increased in steps of 2.5 cmH₂O over a series of 7 steps to a maximum of 20 cmH₂O. The pre-load was kept at each filling pressure for 2 min, during which time the pressure trace could be observed to stabilize and the coronary flow was measured. On completion of the variable pre-load experiment, the variable after-load portion of the experiment was immediately commenced.

Fixed Pre-load and changing After-load:During this part of the experiment the filling pressure (pre-load) was set at 10 cm H₂O and the after-load was then increased from 76 cm H₂O (55.9 mm Hg) in 9 steps; of 2 min duration. The maximum height (after-load) to which each individual heart was ultimately exposed, was determined either by attainment of the maximal available after-load height of 145 cm H₂O (106.66 mm Hg), or the height at which measured aortic flow became 0 ml/min. In the later situation, the heart was considered to have “functionally failed.” To ensure that this failure was indeed functional and not due to other causes (e.g., permanent ischemic or valvular damage) all hearts were then returned to the initial perfusion conditions (pre-load 10 cm H₂O; after-load 75 cm H₂O) for 4 minutes to confirm that pump function could be restored. At the end of this period the hearts were arrested with a retrograde infusion of 4 ml of cold KCL (24 mM). The atria and vascular remnants were then excised, the heart blotted dry and weighed. The ventricles were incised midway between the apex and atrioventricular sulcus. Measurements of the ventricular wall thickness were then made using a micro-caliper (Absolute Digimatic, Mitutoyo Corp, Japan).

Data from the Powerlab was extracted by averaging 1 min intervals from the stable part of the electronic trace generated from each step in the protocol. The results from each group were then combined and analyzed for differences between the groups for the various cardiac function parameters (aortic flow, cardiac flow, MLVDP, LV or aortic +/−dP/dt). Differences between repeated observations at different pre-load conditions were explored and contrasted between study group using a mixed models approach to repeated measures (SAS v8.1, SAS Institute Inc, Cary N.C.). Missing random data were imputed using a maximum likelihood approach. Significant mean and interaction effects were further examined using the method of Tukey to maintain a pairwise 5% error rate for post hoc tests. All tests were two-tailed. Survival analysis was done using Proc Liftest (SAS V8.2). A one-way analysis of variance was used to test for difference between groups in various weight parameters. Tukey's tests were used to compare each group with each other. In each graph unless otherwise stated.* indicates p<0.05=STZ v STZ/D7, #.p<0.05=STZ/D7 v Sham/D7.

Results showing the weights of the animals at the end of the experimental period are found in Table 5. STZ-treated animals were about 50% smaller than their corresponding age matched normals. A graph of the percentage change in weight for each experimental group is found in FIG. 13, wherein the arrow indicates the start of trientine treatment.

Blood glucose values for the three groups of rats are presented in FIG. 14. Generally, the presence of diabetes was established and confirmed within 3-5 days following the

TABLE 5
Initial and final animal body weights (mean ± SD)

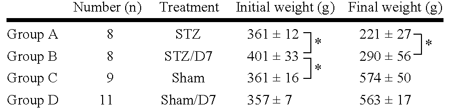

*P < 0.05

STZ injection. The Sham and Sham/D7 control group remained normoglycemic throughout the experiment. Treatment with the trientine made no difference to the blood glucose profile (p=ns) in either treated group compared to their respective appropriate untreated comparison group.

Final heart weight and ventricular wall thickness measurements are presented in Table 6. There was a small but significant improvement in the “heart:body weight” ratio with treatment in the STZ-treated animals. There was a trend toward improved “ventricular wall thickness:bodyweight” ratio in trientine treated STZ-treated rats compared to non-STZ-treated but this did not reach significance.

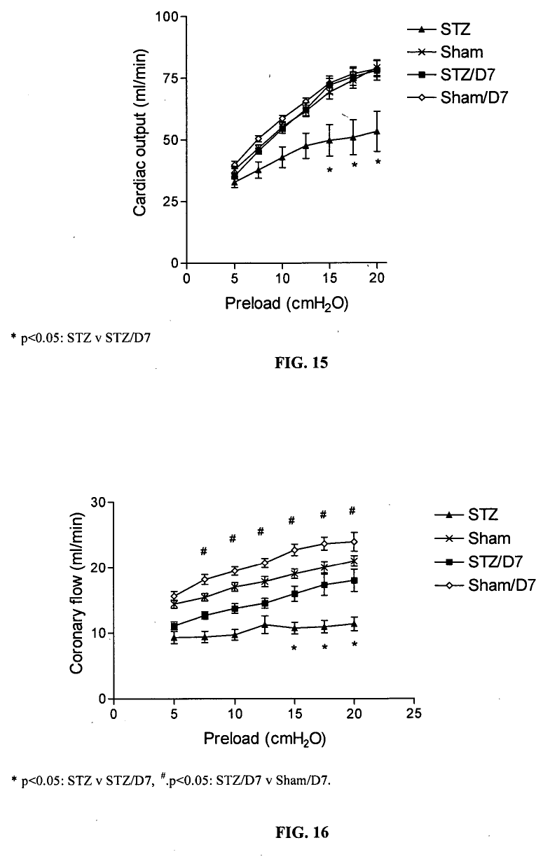

Fixed After-load and changing Pre-load The following graphs of FIGS. 15 to 20 represent cardiac performance parameters of the animals (STZ-treated; STZ-treated +trientine; and sham-treated controls) while undergoing increasing atrial filling pressure (5-20 cmH₂O, pre-load) with a constant after-load of 75 cm H₂O. All results are mean±sem. In each graph for clarity unless otherwise stated, only significant differences related to the STZ/D7 the other groups are shown:* indicates p<0.05 for STZ v STZ/D7, # p<0.05 for STZ/D7 v Sham/D7. Unless stated, STZ/D7 v Sham or Sham/D7 was not significant.

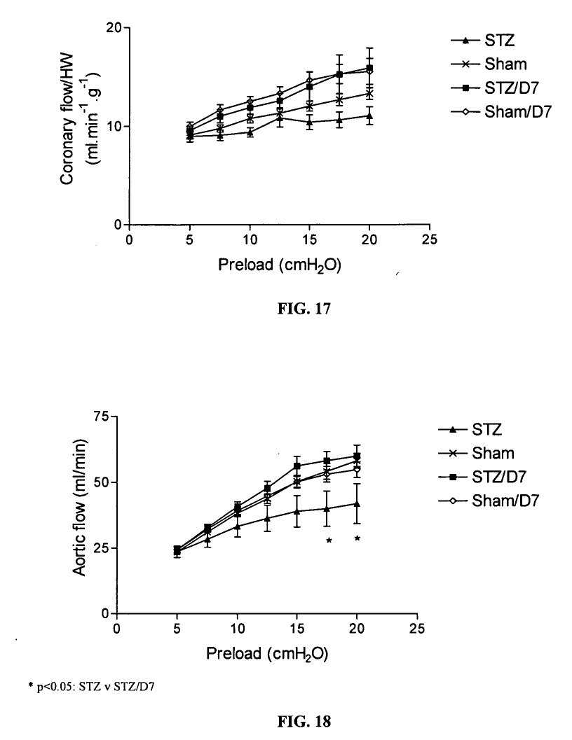

Cardiac output (FIG. 15) is the sum to the aortic flow (FIG. 18) and the coronary flow as displayed in FIG. 16. Since the control hearts and experimental groups have significantly different final weights, the coronary flow is also presented (FIG. 17) as the flow normalized to heart weight (note that coronary flow is generally proportional to cardiac muscle mass and therefore to cardiac weight).

TABLE 6
Final heart weights (g) and per g of animal body Weight (BW) (mean ± SD)

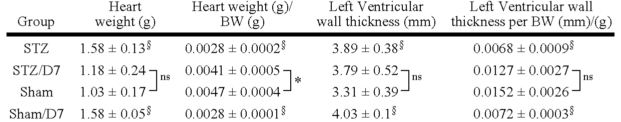

*P < 0.05
§ = significant with the STZ and STZ/D7 groups p < 0.05

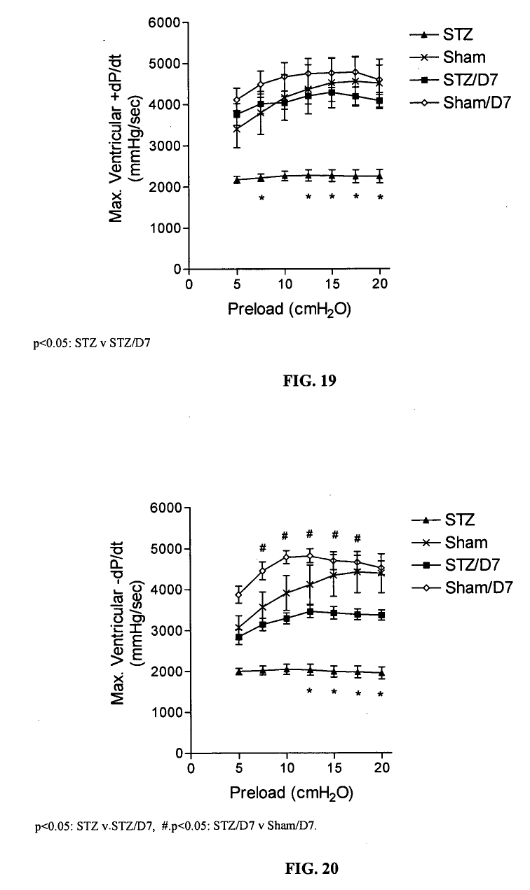

The first derivative of the pressure curve gives the rate of change in pressure development in the ventricle with each cardiac cycle and the maximum positive rate of change (+dP/dt) value is plotted in FIG. 19. The corresponding maximum rate of relaxation (−dP/dt) is in FIG. 20. Similar results showing improvement in cardiac function were found from the data derived from the aortic pressure cannula (results not shown).

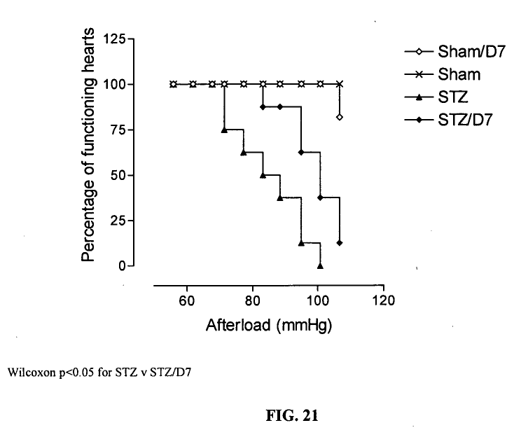

Fixed Pre-load and changing After-load:Under conditions for constant pre-load and increasing after-load the ability of the hearts to cope with additional after-load work was assessed. The plot of functional survival, that is, the remaining number of hearts at each after-load that still had an aortic output of greater than 0 ml/min, is found in FIG. 21.

Administration of trientine improved cardiac function in STZ-treated rats compared to untreated STZ-treated controls. For example, cardiac output, ventricular contraction and relaxation, and coronary flow were all improved in trientine treated STZ-treated rats compared to untreated STZ-treated controls.

### EXAMPLE 6

This Example was carried out to further evaluate the effect of acute trientine administration on tissue repair, in this case on cardiac tissue repair, by assessing left ventricular (LV) histology.

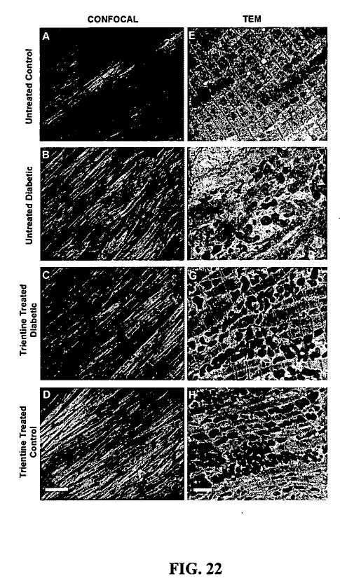

Methods were as follows. Following functional analysis, LV histology was studied by laser confocal (LCM; FIG. 22a-d) and transmission electron microscopy (TEM; FIG. 22e-h). For LCM, LV sections were co-stained with phalloidin to visualize actin filaments, and β₁-integrin as a marker for the extracellular space. Ding B, et al., “Left ventricular hypertrophy in ascending aortic stenosis in mice:anoikis and the progression to early failure,” Circulation 101:2854-2862 (2000).

For each treatment, 5 sections from each of 3 hearts were examined by both LCM and TEM. For LCM, LV sections were fixed (4% paraformaldehyde, 24 h); embedded (6% agar); vibratomed (120 pm, Campden); stained for f-actin (Phalloidin-488, Molecular Probes) and β₁-integrin antibody with a secondary antibody of goat anti-rabbit conjugated to CY5 (1:200; Ding B, et al., “Left ventricular hypertrophy in ascending aortic stenosis in mice:anoikis and the progression to early failure,” Circulation 101:2854-2862 (2000)); and visualised (TCS-SP2, Leica). For TEM, specimens were post-fixed (1:1 v/v 1% w/v 0s0 M 0s0 M PBS); stained (aqueous uranyl acetate (2% w/v, 20 mm) then lead citrate (3 mm)); sectioned (70 nm); and visualized (CM-12, Phillips).

The results were as follows. Copper chelation normalized LV structure in STZ-treated rats. Compared with controls (FIG. 22a), diabetes caused obvious alterations in myocardial structure, with marked loss of myocytes; thinning and disorganization of remaining myofibrils; decreased density of actin filaments; and marked expansion of the interstitial space (FIG. 22b). These findings are consistent with previous reports. Jackson C V, et al., “A functional and ultrastructural analysis of experimental diabetic rat myocardium:manifestation of acardiomyopathy,” Diabetes 34:876-883 (1985). By marked contrast, myocardial histology following trientine treatment was improved (FIG. 22c). Importantly, the orientation and volume of cardiomyocytes and their actin filaments was largely normalized, consistent with the normalization of −dP_{LV}/dt observed in the functional studies. Trientine treatment reversed the expanded cardiac ECM. Myocardium from trientine-treated non-STZ-treated rats appeared normal by LCM (FIG. 22d) suggesting that it has no detectable adverse effects on LV structure. Thus, Cu chelation essentially restored the normal histological appearance of the myocardium without suppressing hyperglycaemia. These data provide important structural correlates for the functional recovery of these hearts, shown above, and support the efficacy of trientine to enhance and/or stimulate tissue repair.

TEM was largely consistent with LCM. Compared with controls (FIG. 22e), diabetes caused unmistakable myocardial damage characterized by loss of myocytes with evident myocytolysis; disorganization of remaining cardiomyocytes in which swollen mitochondria were prominent; and marked expansion of the extracellular space (FIG. 22f). These findings are consistent with previous reports. Jackson C V, et al., “A functional and ultrastructural analysis of experimental diabetic rat myocardium:manifestation of acardiomyopathy,” Diabetes 34:876-883 (1985). Oral trientine caused substantive recovery of LV structure in STZ-treated rats, with increased numbers and normalized orientation of myocytes; return to normal of mitochondrial structure; and marked narrowing of the extracellular space (FIG. 22g). These data suggest that hyperglycaemia-induced systemic Cu^{II} accumulation might contribute to the development of mitochondrial dysfunction. Brownlee M, “Biochemistry and molecular cell biology of diabetic complications,” Nature 414:813-820 (2001). Myocardium from trientine-treated non-STZ-treated rats appeared normal by TEM (FIG. 22h). Thus, trientine treatment normalized both cellular and interstitial aspects of hyperglycaemia-induced myocardial damage. Taken together, these microscopic studies provide remarkable evidence that selective Cu-chelation can substantially improve LV structure, even in the presence of severe chronic hyperglycaemia.

In sum, it was demonstrated that (1) Treatment with trientine had no obvious effect on blood glucose concentrations in the two STZ-treated groups (as expected); (2) There was a small but significant improvement in the (heart weight)/(body weight) ratio in the trientine-treated STZ-treated group compared to that of the untreated STZ-treated group; (3) When the Pre-load was increased with the After-load held constant, cardiac output was restored to Sham values. Both the aortic and absolute coronary flows improved in the trientine treated group; (4) Indicators for ventricular contraction and relaxation were both significantly improved in the trientine treated group compared to equivalent values in the untreated STZ-treated group. The improvement restored function to such an extent that there was no significant difference between the trientine treated and the sham-treated control groups; (5) The aortic transducer measures of pressure change also showed improved function in the trientine treated STZ-treated group compared to the untreated STZ-treated rats (data not shown); (6) When after-load was increased in the presence of constant pre-load, it was observed that the heart's ability to function at higher after-loads was greatly improved in the trientine treated STZ-treated group compared to the untreated STZ-treated group. When 50% of the untreated STZ-treated hearts had failed, about 90% of the trientine treated STZ-treated hearts were still functioning; (7) Compared to the untreated STZ-treated hearts, the response of the trientine treated STZ-treated hearts showed significant improvements in several variables:cardiac output, aortic flow, coronary flow, as well as improved ventricular contraction and relaxation indices; (8) Trientine treatment of normal animals had no adverse effects on cardiac performance; and, (9) Histological observations (TEM and LCM) also showed improvement in cardiac architecture in rats following treatment with trientine.

Treatment of STZ-treated rats with trientine dramatically improves several measures of cardiac function. It is also concluded that administration of oral trientine for 7 weeks in Wistar rats with previously established diabetes of 6 weeks duration resulted in a global improvement in cardiac function. This improvement was demonstrated by improved contractile function (+dP/dT) and a reduction in ventricular stiffness (−dP/dT). The overall ability of the trientine treated heart to tolerate increasing after-load was also substantially improved.

### EXAMPLE 7

This Example was carried out to assess the effect of chronic trientine administration on tissue repair as evidenced by the effect on cardiac structure and function in diabetic and non-diabetic humans.

Methods were as follows. Human studies were approved by institutional ethics and regulatory committees. The absorption and excretion of trientine, and representative plasma concentration-time profiles of trientine after oral administration have been reported (see Miyazaki K, et al., “Determination of trientine in plasma of patients with high-performance liquid chromatography,” Chem. Pharm. Bull. 38:1035-1038 (1990)).

Subjects (30-70 y) who provided written informed consent were eligible for inclusion if they had:T2DM with HbA_{1c}>7%; cardiac ejection fraction (echocardiography)≧45% with evidence of diastolic dysfunction but no regional wall-motion anomalies; no new medications for more than 6 months with no change of β-blocker dose; normal electrocardiogram (sinus rhythm, normal PR Interval, normal T wave and QRS configuration, and isoelectric ST segment); and greater than 90% compliance with single-blinded placebo therapy during a 2-w run-in period. Women were required to be post-menopausal, surgically sterile, or non-lactating and non-pregnant and using adequate contraception. Patients were ineligible if they failed to meet the inclusion criteria or had:morbid obesity (B. M. I.≧45 kg.m⁻²)T1 DM; a history of significant cardiac valvular disease; evidence of autonomic neuropathy; ventricular wall motion abnormality; history of multiple trientine allergies; use or misuse of substances of abuse; abnormal laboratory tests at randomisation; or standard contraindications to MRI.

Before randomization, potentially eligible subjects entered a 4-w single blind run-in phase of two placebo-capsules twice-daily and underwent screening echocardiography, being excluded if regional wall motion abnormalities or impaired LV systolic function (ejection fraction <50%) were detected. In addition, LV diastolic filling was assessed using mitral inflow Doppler (with pre-load reduction) to ensure patients had abnormalities of diastolic filling; no patient with normal mitral filling proceeded to randomisation. Subjects meeting inclusion criteria and with no grounds for exclusion were then randomised to receive trientine (600 mg twice-daily) before meals (total dose 1.2 g.d⁻¹) or 2 identical placebo capsules twice-daily before meals, in a double-blind, parallel-group design. Treatment assignment was performed centrally using variable block sizes to ensure balance throughout trial recruitment and numbered trientine packs were prepared and dispensed sequentially to randomised patients. The double-blind treatment was continued for 6 months in each subject.

At baseline and following 6 months' treatment, LV mass was determined using cardiac MRI, performed in the supine position with the same 1.5 T scanner (Siemens Vision) using a phased array surface coil. Prospectively gated cardiac cine images were acquired in 6 short axis and 3 long axis slices with the use of a segmented k-space pulse sequence (TR 8 ms; TE 5 ms; flip angle 10°; field of view 280-350 mm) with view sharing (11-19 frames.slice⁻¹). Each slice was obtained during a breath-hold of 15-19 heartbeats. The short axis slices spanned the left ventricle from apex to base with a slice thickness of 8 mm and inter-slice gap of 2-6 mm. The long axis slices were positioned at equal 60° intervals about the long axis of the LV. Cardiac MRI provides accurate and reproducible estimates of LV mass and volume. LV-mass and volume were calculated using guide point modeling, which produces precise and accurate estimations of mass and volume. Briefly, a three dimensional mathematical model of the LV was interactively fitted to the epicardial and endocardial boundaries of the LV wall in each slice of the study, simultaneously. Volume and mass were then calculated from the model by numerical integration (mass=wall volume×1.05 g.ml⁻¹). All measurements were performed by 1 measurer at the end of six months' data collection. Outcome analyses were conducted by intention-to-treat, using a maximum likelihood approach to impute missing at random data within a mixed model, and marginal least-squares adjusted-means were determined. Changes from baseline were compared between treatment-groups in the mixed model with baseline values entered as covariate. Since there were only 2 groups in the main effect and no interaction effect, no post hoc procedures were employed. In additional analysis the influence of clinically important differences between the treatment groups at baseline was considered by adjusting for them as covariates in an additional model. All P values were calculated from 2-tailed tests of statistical significance and a 5% significance level was maintained throughout. The effect of treatment on categorical variables was tested using the procedures of Mantel and Haenzel (SAS v8.01, SAS Institute).

Table 7 shows baseline information on 30 patients with long-standing type 2 diabetes, no clinical evidence of coronary artery disease and abnormal diastolic function who participated in a 6-month randomized, double blind, placebo controlled study of chronic oral therapy with trientine dihydrochloride.

TABLE 7
Characteristics of Study Participants

| | | | Trientine | |
|---|---|---|---|---|
| | Placebo | | dihydrochloride | |
| N | 15 | | 15 | |
| Median age (years) | 54 | (range 43-64) | 52 | (range 33-69) |
| % female | 44% | | 56% | |
| Median duration of | 10 | (1-24) | 8 | (1-21) |
| diabetes (years) | | | | |
| Mean body mass index | 32 | (5) | 34 | (5) |
| (kg/m²) (SD) | | | | |
| % hypertensive | 64% | | 80% | |
| Mean % HbA_{1c} (SD) | 9.3 | (1.3) | 9.3 | (2.0) |
| Initial left ventricular | 202.2 | (53.1) | 207.5 | (48.7) |
| mass (g) (SD) | | | | |

Trientine (600 mg twice-daily, a dose at the lower end of those employed in adult Wilson's disease, see Dahlman T, et al., “Long-term treatment of Wilson's disease with triethylene tetramine dihydrochloride (trientine),” Quart. J. Med 88:609-616 (1995)) or placebo was administered orally for 6 months to equivalent groups of diabetic adults (n=15.group⁻¹; Table 7), also matched for pharmacotherapy including: β-blockers, calcium antagonists, ACE-inhibitors, cholesterol-lowering trientines, antiplatelet agents and antidiabetic trientines. LV masses were determined by tagged-molecular resonance imaging (MRI; see Bottini P B, et al., “Magnetic resonance imaging compared to echocardiography to assess left ventricular mass in the hypertensive patient,” Am. J. Hypertens 8:221-228 (1995)) at baseline and following 6 months' trientine treatment. As expected, diabetics initially had significant LVH, consistent with previous reports. Struthers A D & Morris A D, “Screening for and treating left-ventricular abnormalities in diabetes mellitus:a new way of reducing cardiac deaths,” Lancet 359:1430-1432 (2002).

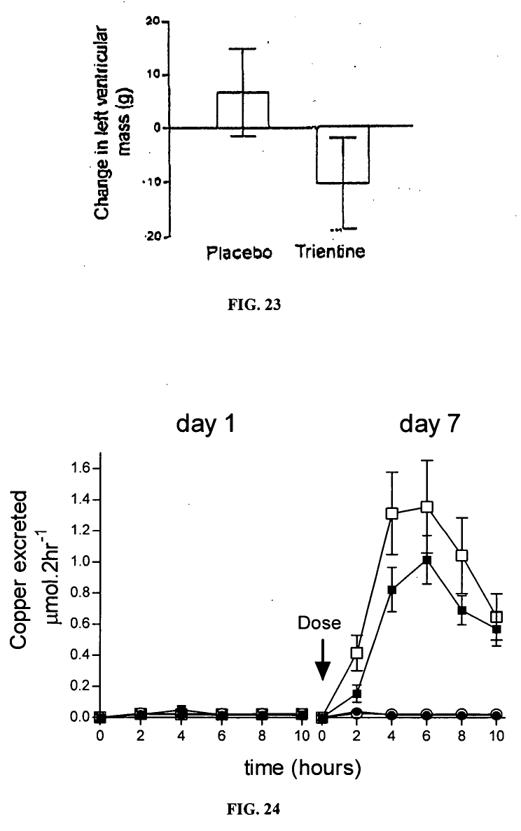

Results showed that Trientine treatment reverses LVH in type-2 diabetic humans. MRI scans of the heart at baseline and 6-months showed a significant reduction in LV mass. Mean LV mass in diabetics significantly decreased, by 5%, following 6 months' trientine treatment, whereas that in placebo-treated subjects increased by 3% (FIG. 23); this highly significant effect remained after LV mass was indexed to body surface area, and occurred without change in systolic or diastolic blood pressure (Table 8). Thus, trientine caused powerful regression in LV mass without altering blood pressure or urinary volume. No significant trientine-related adverse events occurred during the 6 months' trientine therapy.

### Chronic Trientine Treatment Improves Cardiac Structure and Function in Humans

TABLE 8
Results of Trientine treatment

| | Placebo | Trientine-treated |
|---|---|---|
| Δ urinary copper | 0.67 | −0.83 |
| (μmol · L⁻¹) | (−1.16 to 2.49) | (−2.4 to 0.74) |
| Δ systolic blood pressure | −1.9 | −3.5 |
| (mmHg) | (−10.6 to 6.8) | (−9.5 to 1.8) |
| Δ diastolic blood pressure | −4.5 | −3.9 |
| (mmHg) | (−9.0 to 0.01) | (−13.4 to 6.5) |
| Δ left ventricular mass/body | +3.49 | −5.56** |
| surface area | (0.63 to 7.61) | (−9.64 to −1.48) |
| (g · m⁻²) | | |

Differences in key treatment-variables (6 months—baseline, mean (95% confidence interval. *, P<0.05 vs. placebo **, P<0.01 vs. placebo).

MRI scans of the heart at baseline and 6-months showed a significant reduction in LV mass.

In sum, trientine administration for 6 months yielded improvements in tissue repair in humans, for example, in the structure and function of the human heart.

### EXAMPLE 8

This Example was carried out to assess the effect of chronic trientine administration on urinary metal excretion in diabetic and non-diabetic humans.

Methods were as follows. Human studies were approved by institutional ethics and regulatory committees. We measured urinary metal excretion in human males with T2DM or matched non-diabetic controls, baseline information on which is shown in Table 9, in a randomized, double blind, placebo-controlled trial. Males with uncomplicated T2DM (Table 9) underwent 12-d elemental balance studies in a fully residential metabolic unit. All foods and beverages were provided. Total daily intake (method of double diets) and excretion (urinary and fecal) of trace elements (Ca, Mg, Zn, Fe, Cu, Mn, Mo, Cr and Se) were determined (ICP MS). Baseline measurements were taken during the first 6 d, after which oral trientine (2.4 g once-daily) or matched placebo was administered in a 2×2 randomized double-blind protocol and metal losses measured for a further 6 d.

TABLE 9
Characteristics of Study Participants

| | Placebo | Trientine | Placebo | Trientine |
|---|---|---|---|---|
| | control | treated control | diabetic | treated diabetic |
| Median age (years) | 42 | 52 | 51 | 50 |
| | (range 32-53) | (range 30-68) | (range 32-66) | (range 30-64) |
| n | 10 | 10 | 10 | 10 |
| Median duration of | — | — | 5.9 | 7.5 |
| diabetes (years) | | | (range 1-13) | (range 1-34) |
| Fasting plasma | 4.7 ± 0.3 | 5.0 ± 0.4 | 11.5 ± 3.8 | 10.8 ± 4.3 |
| glucose (mmol · L⁻¹) | | | | |
| Mean HbA_{1c} (%) | 5.4 ± 0.2 | 5.0 ± 0.3 | 9.9 ± 2.7 | 9.1 ± 1.6 |
| Body mass index | 24.6 ± 3.5 | 27.9 ± 5.2 | 32.9 ± 4.5 | 30.4 ± 3.1 |
| (kg · m⁻²) | | | | |

(mean ± S.E.M. unless otherwise stated);
f.b.g., HbA_{1c} and B.M.I. were significantly greater in diabetics and groups were otherwise well-matched).

Results showed that urinary Cu losses are increased following oral trientine treatment in humans with type-2 diabetes. Urine volumes were equivalent in trientine- and placebo-treated groups. Basal 2-h Cu-losses were measured for 10 h in diabetic (n=20) and matched control (n=20) subjects during part of day I; and daily losses were determined throughout days 1-6.

Baseline urinary Cu-excretion was significantly greater in diabetics than controls (mean diabetic, 0.257 μmol.d⁻¹ control, 0.196; P<0.001).

Trientine- and placebo-evoked 2-h urinary Cu-excretion was measured again in the same subjects on day 7 following oral trientine (2.4 g once-daily) or matched placebo (n=10.group⁻¹. Trientine increased urinary Cu in both groups, but the excretion rate in diabetes was greater (FIG. 24; P<0.05). There was no corresponding increase in trientine-evoked urinary Fe excretion, although basal concentrations in diabetes were increased relative to control (P<0.001; results not shown). Thus, trientine elicited similar urinary Cu responses in rats with T1DM and in humans with T2DM. Mean trientine-evoked urinary Cu-excretion was 5.8 μmol.d⁻¹ in T2DM compared to 4.1 μmol.d⁻¹ in non-diabetic controls, a 40% increase.

In sum, chronic trientine administration increased urinary copper in both diabetic and nondiabetic groups, but the excretion rate in diabetics was greater. No corresponding increase in urinary Fe excretion was observed with trientine. Thus, trientine elicited similar urinary copper responses in rats with type 1 diabetes mellitus and in humans with type 2 diabetes mellitus.

### EXAMPLE 9

This Example was carried out to determine the effect of oral trientine administration on fecal output of metals in diabetic and non-diabetic humans. Methods were as follows.

Oral trientine (2.4 g once daily) or matched placebo were administered to matched groups (n=10/group) of humans with type-2 diabetes mellitus (T2DM) or matched controls. Total metal balance studies were performed in a residential metabolic unit. Total fecal outputs were collected daily for 12 days, freeze dried, and analyzed by ICP-MS for content of Cu, Fe, Zn, Ca, Mg, Mn, Cr, Mb and Se. Baseline measurements were taken during the first 6 d after which oral trientine or matched placebo were administered in a 2×2 randomized double-blind protocol and metal losses measured for a further 6 d.

Results were as follows. Mean daily fecal losses of Cu were not significantly different between subjects before and after administration of trientine or placebo, nor were Cu outputs different between diabetic and control subjects. The lack of effect of trientine on fecal Cu output was unexpected (see Table 11), and contrasts sharply with reports from Wilson's disease, in which trientine reportedly increased fecal Cu excretion.

TABLE 11
Fecal copper excretion

| Mean CU Losses (mg/day) | Pre-Tment | Post-Tment |
|---|---|---|
| Diab-Plac (n = 10) | 1.914503965 | 1.937921277 |
| Ctrl-Plac (n = 10) | 1.670142101 | 2.078654892 |
| Diab-Drug (n = 10) | 1.869867293 | 1.965342334 |
| Ctrl-Drug (n = 10) | 2.19850868 | 2.045467014 |
| SEM: Diabetic-PrePlac | 0.122570307 | 0.178995736 |
| SEM: Control-PrePlac | 0.1765707 | 0.209400786 |
| SEM: Diabetic-PreDrug | 0.228263465 | 0.144463056 |
| SEM: Control-PreDrug | 0.209289978 | 0.124516832 |

| Reference Values | | |
|---|---|---|
| Ishikawa et al (2001): control | ˜1.00 | mg/d |
| Kenzie Parnall et al (1998): control | ˜1.30 | mg/d |
| Kosaka H et al (2001): control | 53.5 | ug/d |

Results of fecal output studies of other metals were similar. Neither diabetes nor trientine had measurable effects on outputs of Zn, Fe, Ca, Mg, Mn, Cr, Mb or Se. In sum, in normal humans and those with T2DM, trientine did not increase fecal output of Cu or other metals. Therefore, trientine does not act in T2DM by increasing fecal Cu output. On the other hand, our previous results showed that trientine administration increased urinary Cu output. Taken in aggregate, these results indicate that trientine acts to remove Cu from the systemic compartment by increasing its excretion in the urine. Therefore, systemically active forms of trientine are the preferred embodiment of this invention.

The human data, taken together with those in rats above, indicate that chronic Cu chelation can cause significant tissue regeneration. Trientine largely reversed heart failure and LV damage in severely diabetic rats. Furthermore, six months' oral trientine administration significantly ameliorated left ventricular hypertrophy in humans with type-2 diabetes. These data also show that increased systemic Cu^{II} can be removed by treatment with the Cu-selective chelator, trientine.

### EXAMPLE 10

This Example assessed the effect of the copper chelation efficacy of various concentrations of parenteral administration of trientine on anaesthetized STZ-treated and non-STZ-treated male Wistar rats through the measurement of copper in the urine.

Stock solutions of various intravenous formulations having concentrations of trientine hydrochloride were made up in 0.9% saline and was stored for four months at 4° C. without appreciable deterioration in efficacy. The concentrations of the stock formulations were:0.67 mg/ml, 6.7 mg/ml, 67 mg/ml, and 670 mg/ml. The formulation was then administered to the rats in doses of 0.1 mg/kg, 1 mg/kg, 10 mg/kg, and 100 mg/kg to the animals respectively.

Six to seven weeks (mean=44±1 days) after administration of STZ, animals underwent either a control or trientine experimental protocol. All animals were fasted overnight prior to surgery but continued to have ad libitum access to deionized water. Induction and maintenance of surgical anesthesia was by 3-5% halothane and 2 l.min⁻¹ O2. The femoral artery and vein were cannulated with a solid-state blood pressure transducer (Mikrotip™ 1.4F, Millar Instruments, Texas, USA) and a saline filled PE 50 catheter respectively. The ureters were exposed via a midline abdominal incision, cannulated using polyethylene catheters (external diameter 0.9 mm, internal diameter 0.5 mm) and the wound sutured closed. The trachea was cannulated and the animal ventilated at 70-80 breaths.min⁻¹ with air supplemented with O2 (Pressure Controlled Ventilator, Kent Scientific, Conn., USA). The respiratory rate and end-tidal pressure (10-15 cmH2O) were adjusted to maintain end-tidal CO2 at 35-40 mmHg (SC-300 CO2 Monitor, Pryon Corporation, Wisconsin, USA). Body temperature was maintained at 37° C. throughout surgery and the experiment by a heating pad. Estimated fluid loss was replaced with intravenous administration of 154 mmol.l⁻¹ NaCl solution at a rate of 5 ml.kg⁻¹.h⁻¹.

Mean arterial pressure (MAP), heart rate (HR, derived from the MAP waveform) oxygen saturation (Nonin 8600V Pulse Oximeter, Nonin Medical Inc., Minnesota, USA) and core body temperature, were all continuously monitored throughout the experiment using a PowerLab/16s data acquisition module (AD Instruments, Australia). Calibrated signals were displayed on screen and saved to disc as 2 s averages of each variable.

Following surgery and a 20 min stabilization period, the experimental protocol was started. The trientine formulation or an equivalent volume of saline was intravenously administered hourly in doses of increasing strength from 0.1 mg/kg, 1.0 mg/kg, 10 mg/kg, and 100 mg/kg. Urine was collected throughout the experiment in 15 min aliquots.

Sample pretreatment was carried out as follows. Urine:Urine was collected in pre-weighed 1.5 ml micro test tubes (eppendorf). After reweighing, the urine specimens were centrifuged and the supernatant diluted 25:1 with 0.02 M 69% Aristar grade HNO₃. The sample was stored at 4° C. prior to GF-AAS analysis. If it was necessary to store a sample for a period in excess of 2 weeks, it was frozen and kept at −20° C. Serum:Terminal blood samples were centrifuged and serum treated and stored as per urine until analysis. From the trace metal content of serum from the terminal blood sample and urine collected over the final hour of the experiment, renal clearance was calculated using the following equation:
renal clearance of trace metal (μl.min⁻¹) concentration of metal in urine (μg.μl⁻¹)*rate of urine flow (μl.min⁻¹) concentration of metal in serum (μg.μl⁻¹)

Statistical analyses were carried out as follows. All values are expressed as mean±SEM and P values <0.05 were considered statistically significant. Student's unpaired t-test was initially used to test for weight and glucose differences between the STZ-treated and control groups. For comparison of responses during trientine exposure, statistical analyses were performed using analysis of variance (Statistics for Windows v.6.1, SAS Institute Inc., California, USA). Subsequent statistical analysis was performed using a mixed model repeated measures ANOVA design (see Example 4).

The results were as follows. With regard to the cardiovascular effects there were no adverse effects from the acute injection of trientine. See FIG. 25 that shows no adverse cardiovascular effects after the injection, although at 100 mg/kg this gave a transient drop in blood pressure. This change was a maximum blood pressure fall of 19+/−4 mmHg, however the rat recovered in 10 minutes (not shown).

In summary, acute intravenous administration of trientine in the concentration ranges from between 0.1 mg/kg, 1 mg/kg, 10 mg/kg, and 100 mg/kg has no significant effect on blood pressure. Furthermore, a trientine formulation is efficacious as a copper chelator when given intravenously and that trientine in saline remains active as a copper chelator after storage at 4° C. for 4 months.

### EXAMPLE 11

This Example assessed the stability of a stored trientine formulation by its ability to chelate copper.

A standard 100 mM solution of Trientine HCl was made up in deionized (MilliQ) water. One sample of the solution was stored in the dark at 4° C. and 21° C. in the dark and a third sample was stored at 21° C. in daylight.

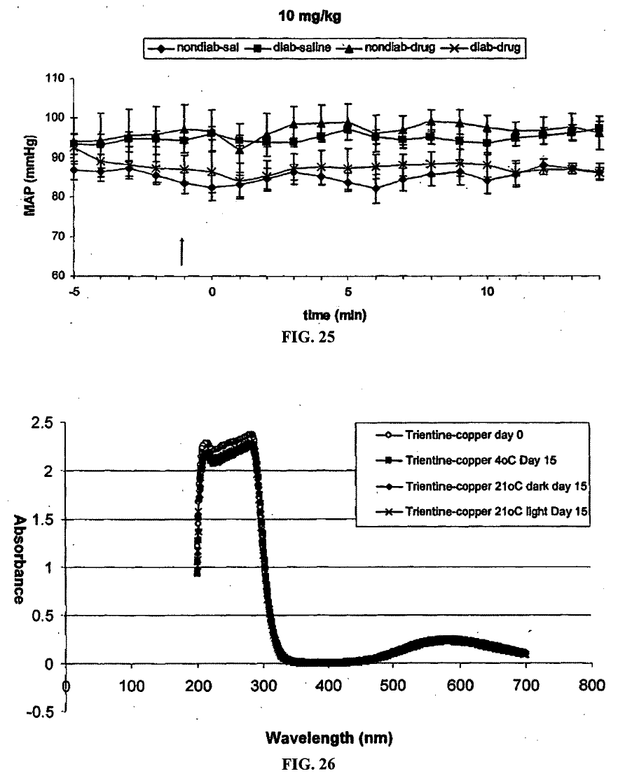

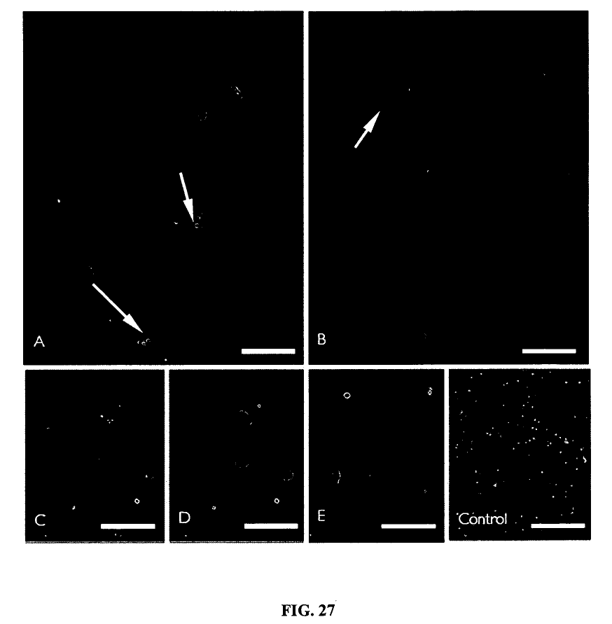

The Ultraviolet-visible spectrum of the formulation was initially measured at day 0 and then at day 15. 20 μl aliquots of sample solutions were taken at day 15. For each aliquot 960 μl of 50 mM TRIS buffer and 20 μl aliquot of Copper Nitrate standard (100 mM—Orion Research Inc) were added. This was then measured over wavelengths 700-210 nm to determine the binding stability of the trientine formulations. See FIG. 26 that shows that there was no detectable change in the ability of the trientine formulation to chelate copper over this 15 day time period irrespective of storage conditions. Furthermore room light had no detectable detrimental effect on copper chelation and that trientine is stable as a chelator while in solution.

### EXAMPLE 12

In this Example cortical neuronal cultures were grown from 21 day old postnatal male Wistar rat brain cells. These rats were raised on Teklad 2018 vegetarian rat chow before sacrifice. The cells were then grown on poly-D-lysine coated glass cover slips for two weeks in growth media containing foetal bovine serum (Brewer et. al., 1993). All procedures used were fully approved by the University of Auckland animal ethics committee.

The cultures were then washed and fixed using neutral buffered formalin. Antibodies for bovine serum albumin were then used to determine whether bovine serum albumin could be detected intracellularly of the cells. Both the neuron and astrocyte cells had internalised BSA and this is more clearly seen in FIG. 27.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member or subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

1. A method of treating a subject having a neurodegenerative disorder, comprising administering a pharmaceutically acceptable copper antagonist in an amount effective to increase copper output in the urine of said subject.

2. A method of treating a subject having a neurodegenerative disorder, comprising administering a pharmaceutically acceptable copper antagonist in an amount effective to decrease copper uptake in the gastrointestinal tract.

3. Use of a therapeutically effective amount of a pharmaceutically acceptable copper antagonist in the manufacture of a medicament for the treatment of a subject having or suspected of having or predisposed to a neurodegenerative disorder.

4. A method or use as claimed in any of claims 1, 2 or 3 wherein said neurodegenerative disorder is selected from any one or more of the following; dementia, memory impairment caused by dementia, memory impairment seen in senile dementia, various degenerative diseases of the nerves including Alzheimer's disease, Huntingtons disease, Parkinson's disease, parkinsonism, amyotrophic lateral sclerosis (ALS), Friedreich's ataxia and other hereditary ataxia, other diseases, conditions and disorders characterized by loss, damage or dysfunction of neurons including transplantation of neuron cells into individuals to treat individuals suspected of suffering from such diseases, conditions and disorders, any neurodegenerative disease of the eye, including photoreceptor loss in the retina in patients afflicted with macular degeneration, retinitis pigmentosa, glaucoma, and similar diseases, stroke, cerebral ischemia, head trauma, migraine, depression, peripheral neuropathy, pain, cerebral amyloid angiopathy, nootropic or cognition enhancement, multiple sclerosis, ocular angiogenesis, corneal injury, macular degeneration, tumor invasion, tumor growth, tumor metastasis, corneal scarring, scleritis, motor neuron and Lewy body disease, attention deficit disorder, migraine, narcolepsy, psychiatric disorders, panic disorders, social phobias, anxiety, psychoses, obsessive-compulsive disorders, obesity or eating disorders, body dysmorphic disorders, post-traumatic stress disorders, conditions associated with aggression, drug abuse treatment, or smoking secession, traumatic brain and spinal cord injury, and epilepsy.

5. A method as claimed in any of claims 1, 2 or 3 wherein said copper antagonist is a trientine.

6. A method as claimed in any of claims 1, 2 or 3 wherein said copper antagonist is trientine salt.

7. A method as claimed in any of claims 1, 2 or 3 wherein said copper antagonist is a compound of Formula I or II.

8. A method as claimed in any of claims 1, 2 or 3 wherein said copper antagonist is a trientine prodrug.

9. A method as claimed in any of claims 1, 2 or 3 wherein said copper antagonist is an active metabolite of trientine.

10. A method as claimed in claim 9 wherein said metabolite is N-acetyl trientine.

11. Use of a therapeutically effective amount of a pharmaceutically acceptable copper antagonist in the manufacture of a dosage form for the treatment of a subject having or suspected of having or predisposed to a neurodegenerative disease, disorder, and/or condition.

12. A use as claimed in claim 11 wherein said dosage form is any one or more of the following; a transdermal patch, pad, wrap, bandage, and/or device.
